(19) United States
(12) Patent Application Publication
(10) Pub. No.: US 2026/0137700 A1
(43) Pub. Date: May 21, 2026

(54) PHARMACEUTICAL COMBINATION OF PRMT5 INHIBITOR AND DNA DAMAGE-INDUCING SUBSTANCE

(71) Applicant: SK Biopharmaceuticals Co., Ltd., Seongnam-si, Gyeonggi-do (KR)

(72) Inventors: Mijin MOON, Seongnam-si, Gyeonggi-do (KR); So Young KI, Seongnam-si, Gyeonggi-do (KR); Jungtae NA, Seongnam-si, Gyeonggi-do (KR); Ho Yeon LEE, Seongnam-si, Gyeonggi-do (KR); Ilkyoo KOH, Seongnam-si, Gyeonggi-do (KR); Beomjin HONG, Seongnam-si, Gyeonggi-do (KR); Sookkyung PARK, Seongnam-si, Gyeonggi-do (KR); Yong Je SHIN, Seongnam-si, Gyeonggi-do (KR)

(21) Appl. No.: 19/128,593

(22) PCT Filed: Nov. 10, 2023
(86) PCT No.: PCT/KR2023/018109

§ 371 Date: Aug. 19, 2025

(30) Foreign Application Priority Data
Nov. 10, 2022 (KR) 10-2022-0149975
Apr. 13, 2023 (KR) 10-2023-0049041

(51) Int. Cl. A61K 31/553; A61K 9/00; A61K 45/06; A61P 35/00

(52) U.S. Cl. CPC A61K 31/553; A61K 9/0053; A61K 45/06; A61P 35/00

(57) ABSTRACT

The present disclosure pertains to a pharmaceutical combination for treating and/or preventing cancer and a method and use thereof. Specifically, the present disclosure involves a method for inhibiting tumor growth by combining a substance inhibitory of the activity of protein arginine methyltransferase and a drug that is similar to or different from the substance in terms of mechanism, and/or a pharmaceutical combination useful for reducing tumor volume using same. Particularly, a pharmaceutical combination comprising a PRMT5 inhibitor and a DNA damage-inducing substance is provided.

### TECHNICAL FIELD

The present invention relates to a method for treating cancer, and a pharmaceutical combination useful for such treatment and to methods and uses thereof. The present invention relates to a pharmaceutical combination comprising a Protein Arginine Methyltransferase 5 (PRMT5) inhibitor and a DNA damage inducing substance.

### BACKGROUND ART

Cancer is a disease whose mortality rate continues to increase annually due to changes in environmental changes, aging population, and changes in lifestyle. It is known to be one of the top five causes of death worldwide. Treatments for cancer include surgical resection, radiotherapy, and pharmacotherapy, but the best approach to overcome resistance to anticancer agents is to apply drug therapy in combination with existing drugs. Such combination therapy is to overcome resistance to anticancer agent and maximize therapeutic efficacy, by utilizing drugs that act through different mechanisms.

The first-line treatment for lung cancer is currently the combination therapy with platinum-based third-generation anticancer agents such as paclitaxel, docetaxel, vinorelbine, gemcitabine or tecan as the most fundamental therapy. However, there is no significant difference in survival rates among these therapeutic agents, and the reported survival is approximately 10 months (Reference: N Engl J Med 2002; 346:92-8). In case of targeted therapies, erlotinib (Tarceva), gefitinib (Iressa), afatinib (Giotrif) and the like, which inhibit EGFR activity, are also used as first-line treatments. However, they are only used as treatments for non-small cell lung cancer (NSCLC) patients with EGFR expression of about 2%, and only shows a response rate of approximately 50 to 70%, and the remaining 30 to 50% of patients do not respond, so their efficacy is limited (Reference: Journal of Clinical Oncology 2011; 29:21). Even in about 20% of patients with KRAS mutations, the use of sotorasib (KRAS G12C) has shown a response rate of only 25-50% (References: Signal Transduction and Targeted Therapy 2021; 6:386; Clin Cancer Res 2022 Apr. 15; 28(8)).

Breast cancer is the most commonly diagnosed cancer among women (Reference: CA Cancer J Clin. 2021; 71(1):7-33). Approximately 13% of breast cancer patients are known to have increased expression of human epidermal growth factor receptor type 2 (HER2). About 66.6% of breast cancer patients are estrogen receptor (ER)-positive and HER2-negative, while 10.6% of the patients are triple-negative breast cancer (TNBC) patients (ER-negative, progesterone receptor(PR)-negative, and HER2-negative) (Reference: Cancer Epidemiol Biomarkers Prev. 2018; 27(6):619-626). For metastatic breast cancer, HER2-targeted therapies (e.g., trastuzumab) have been developed and widely used. However, since HER2 inhibition alone is not enough to prevent resistance, research is being conducted to maximize therapeutic effects by inhibiting interactions with various signaling systems that affect the resistance mechanism of HER2 breast cancer. (References: J Cell Physiol. 2020 April; 235(4):3142-3156; Mol Cancer Ther. 2017 Jun; 16(6):1145-1154). In addition, for TNBC lacking therapeutic targets such as HER2 or hormone receptors, systemic chemotherapy is typically used due to the absence of targeted treatment options. Cytotoxic anticancer agents such as platinum-based doublet therapies (e.g., carboplatin, cisplatin), taxanes (e.g., paclitaxel), and antimetabolites (e.g., capecitabine, gemcitabine) are employed (Reference: NCCN Guidelines for Patients for metastatic Breast Cancer, 2020).

Glioblastoma is the most common primary malignant brain tumor, and it is known for its extremely poor prognosis with a median survival of less than 18 months after diagnosis (Reference: Front Cell Neurosci. 2017 Oct. 13; 11:318). Due to its aggressive characteristics such as tumor location, hypervascularity, and high invasiveness, glioblastoma remains difficult and limited to treat, even when combining surgical intervention and radiation therapy with multiple chemotherapeutic agents, which often lead to significant side effects and complications (Reference: Clin J Oncol Nurs. 2016 Oct. 1; 20(5 Suppl):S2-8). Among chemotherapeutic agents, temozolomide, which easily crosses the blood-brain barrier (BBB), is mainly used, but the average survival rate is difficult to exceed 14.5 months (Reference: Clin Med Insights Oncol. 2016 Aug. 17; 10:77-82).

In December 2014, based on the observation that cells deficient in the homologous recombination (HR) repair pathway, such as Breast Cancer susceptibility Gene (BRCA) 1-null cancer cells, are vulnerable to deficiencies in poly(ADP-ribose) polymerase (PARP), PARP inhibitors have been developed to treat cancers having homologous recombination deficiencies (HRD). However, the use of PARP inhibitors has been approved only for patients with certain tumor types, such as high-grade serous ovarian cancer or triple-negative breast cancer, or tumor s that may belong to molecular subtypes associated with BRCA 1/2 mutations (e.g., BRCA1/2-mutant breast, ovarian, pancreatic, or prostate cancers). Moreover, the requirement for HR pathway deficiency and the rapid development of resistance (many tumors being initially responsive to PARP inhibitor recover HR pathway function or activate alternative repair pathways via compensatory mutations) limit their application to only specific tumor types.

The PRMT5 protein is involved in cellular proliferation and growth through the methylation of histone and non-histone proteins [Reference: Trends Biochem Sci. 2011; 36(12):633-641], and it is known to be overexpressed in various types of cancers including lymphoma, glioma, breast cancer, and lung cancer [References: Cancer Res 2014, 74(6):1752; Cancer Lett 2018, 427:3; Oncotarget. 2019; 10(34):3151-3153]. Overexpression of PRMT5 is closely associated with poor patient survival such as tumorigenesis and drug resistance, and the like [References: Cell Mol Life Sci. 2015; 72:2041; Oncotarget. 2019; 10(34):3151-3153]. Loss or inhibition of PRMT5 has been shown to impair not only DNA repair but also disrupt the splicing environment, leading to reduced transcription of proliferation-associated genes and defects in cell cycle progression [References: Cell Rep 2019, 26:2316; Cancer Cell 2017, 32:411; Cell Mol Life Sci. 2015; 72:2041].

Therefore, it is necessary to overcome the drug resistance and unmet medical needs that have been pointed out as limitations of existing anticancer drugs, through combination therapy with PRMT5 inhibitors and achieve effective cancer treatment effects.

### DETAILED DESCRIPTION OF THE INVENTION

### Technical Problem

The invention described and claimed herein has many features and embodiments including, but not limited to, those set forth, described, or referenced in this summary. It is not intended to be all-inclusive, and the invention described and claimed herein is not limited to or by the features or embodiments identified in this summary, which are included for illustrative purposes only and are not intended to limit.

In view of the above-described problems, an aspect of the present disclosure relates to a pharmaceutical combination including a PRMT5 inhibitor and a DNA damage inducing substance (for example, at least one DNA damage inducing substance selected from the group consisting of alkylating agents, anti-metabolites, topoisomerase inhibitors, microtubule inhibitors, and PARP inhibitors), for use in the treatment and/or prevention of cancer.

### Technical Solution

The use of specific drugs in this specification is not intended to be limited to those drugs, but is used as representative drugs of PRMT5 inhibitors or DNA damage inducing substances used in combination therewith.

The specific examples described in the specification are not limited to a PRMT5 inhibitor and a single combination drug in combination with it, and the combination drug may be varied. The examples described are part of experiments that were intentionally conducted to include the content within the scope of the appended claims, and may include additional combinations of various drugs.

Each embodiment described herein comprises distinct components and features that may be readily separated from or combined with other features. The contents described in the present specification fall within the scope of the appended claims.

Some compounds as PRMT5 inhibitors according to an embodiment of the present invention, may be any PRMT5 inhibitor other than the compounds of Chemical Formula 1, and may be combined with certain DNA damage inducing substances for treating a cancer. The DNA damage inducing substances for treating a cancer may be selected, for example, from the group consisting of alkylating agents, anti-metabolites, topoisomerase inhibitors, microtubule inhibitors, and poly(ADP-ribose) polymerase (PARP) inhibitors.

The PRMT5 inhibitor according to the present invention is a type II PRMT inhibitor, and can promote monomethylating and asymmetric demethylation by using S-adenosylmethionine (SAM) as a substrate. The PRMT5 inhibitor of the present invention may inhibit PRMT5 competitively or uncompetitively with SAM, and as an example, it may inhibit PRMT5 in an uncompetitive manner with SAM.

The pharmaceutical combination according to the present invention may exhibit a characteristics of reduced side effects or toxicity, and may have a synergistic effect of higher anticancer effect compared to when each agent is administered alone.

As a pharmaceutical combination for treating and/or preventing cancer in a subject, the pharmaceutical combination comprises a PRMT5 inhibitor of Chemical Formula 1, or an optical isomer, stereoisomer or isotopic variant thereof, or a pharmaceutically acceptable salt thereof; and a DNA damage inducing substance.

In the present specification, the DNA damage inducing substances that can be used in combination with the PRMT5 inhibitor may be selected from the group consisting of alkylating agents, anti-metabolites, topoisomerase inhibitors, microtubule inhibitors, and poly (ADP-ribose) polymerase (PARP) inhibitors. The DNA damage inducing substance may be a DNA damage inducing anticancer agent as an anticancer agent for treating a cancer.

The alkylating agents may be selected from the group consisting of cisplatin, carboplatin, oxaliplatin, mechlorethamine, cyclophosphamide, ifosfamide, melphalan, chlorambucil, carmustine (BCNU), lomustine (CCNU), nimustine, altretamine, busulfan, dacarbazine, procarbazine, temozolomide, thiotepa, and lurbinectedin.

The anti-metabolites may be selected from the group consisting of fluorouracil (5-FU), capecitabine, cytarabine, gemcitabine, methotrexate, and mercaptopurine (6-MP).

The topoisomerase inhibitors may be selected from the group consisting of etoposide, teniposide, topotecan, irinotecan, SN-38, dactinomycin, doxorubicin, daunorubicin, mitomycin, and bleomycin.

The microtubule inhibitor may be selected from the group consisting of vinblastine, vincristine, vinorelbine, paclitaxel, and docetaxel.

The PARP inhibitor may be selected from the group consisting of olaparib, veliparib, niraparib, pamiparib, talazoparib, rucaparib, CEP-9722, E7016 (GPI 21016), fluzoparib, AZD5305, and AZD9574.

The subject includes subject with multiple drug resistance, or subject that have a remittance or a low responsiveness to various DNA damage inducing substances.

The subject includes a subject that is either Homologous Recombination Deficiency (HRD)-positive or HRD-negative.

In the case of PARP inhibitors, they show responsiveness only in HRD-positive subjects, but very low responsiveness in HRD-negative subjects. However, by combined use of a PRMT5 inhibitor and a PARP inhibitor, an effect of showing high responsiveness not only in HRD-positive subjects but also in HRD-negative subjects can be achieved. Example of the pharmaceutical combination may be a pharmaceutical combination comprising a poly (ADP-ribose) polymerase (PARP) inhibitor for treating and/or preventing a cancer in a subject having either HRD-positive or HRD-negative.

The cancers may be selected from the group consisting of acoustic neuroma, adenocarcinoma, adrenal cancer, anal cancer, angiosarcoma, benign monoclonal gammopathy, cholangiocarcinoma, bladder cancer, breast cancer, brain cancer, lymphoma, multiple myeloma, lacrimal gland tumor, bronchial cancer, cervical cancer, craniopharyngioma, colorectal cancer, epithelial carcinoma, ependymoma, adenoid cystic carcinoma (ACC), endothelial sarcoma, endometrial cancer, esophageal cancer, Barrett's adenocarcinoma, Ewing's sarcoma, eye cancer, gallbladder cancer, gastric cancer, colon cancer, gastrointestinal stromal tumor (GIST), head and neck cancer, oral cancer (OSCC), throat cancer, hematologic system cancer, hemangioblastoma, inflammatory myofibroblastic tumor, immunocyte-associated amyloidosis, kidney cancer, renal cell carcinoma, liver cancer, lung cancer, melanoma, uveal melanoma, acute myeloid leukemia (AML), myelodysplastic syndrome (MDS), mesothelioma, myeloproliferative disorder (MPD), chronic idiopathic myelofibrosis, chronic myeloid leukemia (CML), chronic neutrophilic leukemia (CNL), neuroblastoma, neurofibroma, neuroendocrine cancer, osteosarcoma, ovarian cancer, papillary adenocarcinoma, pancreatic cancer, penile cancer, prostate cancer, rectal cancer, rhabdomyosarcoma, salivary gland cancer, skin cancer, small intestinal cancer, soft tissue sarcoma, thyroid cancer, urethral cancer, vaginal cancer, and vulvar cancer.

The brain cancer may be selected from the group consisting of meningioma, glioma, medulloblastoma, glioblastoma, or brain metastases. The lung cancer may be selected from the group consisting of non-small cell lung cancer (NSCLC) and small cell lung cancer (SCLC).

The breast cancer or ovarian cancer may be a BRCA1 wild-type cancer, a BRCA1 mutant cancer, a BRCA2 wild-type cancer, a BRCA2 mutant cancer, or may be any one cancer selected from the group consisting of BRCA1 wild-type and BRCA2 wild-type cancer, BRCA1 wild-type and BRCA2 mutant cancer, BRCA1 mutant and BRCA2 wild-type cancer, and BRCA1 mutant and BRCA2 mutant cancer. In a specific example, the cancer may be a HRD-positive or HRD-negative cancer.

According to an embodiment disclosed herein, the present invention relates to a pharmaceutical combination including a combination of a PRMT5 inhibitor and a DNA damage inducing substance for use in the treatment and/or prevention of cancer, wherein the PRMT5 inhibitor is a compound of Chemical Formula 1, or an optical isomer, a stereoisomer or an isotope variant thereof, or a pharmaceutically acceptable salt thereof:

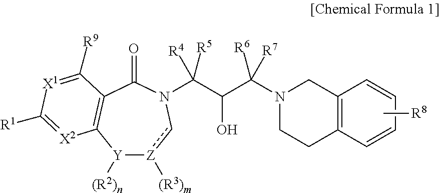

wherein,
X¹ and X² are each independently carbon or nitrogen;
Y is carbon, oxygen or nitrogen;
Z is carbon;
n is an integer of 0 or 1;
m is an integer from 0 to 2;
[symbol] is single bond or double bond;
R¹ is -D-R¹⁰; wherein D is a direct bond, —O—, —C(═O)—, —C≡C— or —CR¹¹R¹²—; R¹⁰ is hydrogen, halo, hydroxy, cyano, alkyl, hydroxyalkyl, haloalkyl, haloalkylsulfonate, dialkylamino, alkylaminoalkyl, dialkylaminoalkyl, dialkylaminocarbonylalkyl, saturated or unsaturated carbocyclyl, saturated or unsaturated heterocyclyl, saturated or unsaturated carbocyclyl-alkyl, or saturated or unsaturated heterocyclyl-alkyl; R¹¹ and R¹² are each independently hydrogen, hydroxy or alkyl; the carbocycle or heterocycle may be substituted with one or more substituents selected from hydroxy, halo, oxo, formyl (—CHO), nitrile, alkyl, alkoxy, hydroxyalkyl, hydroxyhaloalkyl, alkoxyalkyl, haloalkyl, nitrilealkyl, alkylcarbonyl, alkylthiocarbonyl, alkoxycarbonyl, haloalkylcarbonyl, carbocyclyl, carbocyclylcarbonyl, (alkyl)(haloalkyl)amino, (alkyl)(heterocyclyl)amino, heterocyclyl and heterocyclyl-alkyl;
R² is hydrogen or alkyl;
R³ is hydrogen or alkyl;
R⁴, R⁵, R⁶ and R⁷ are each independently hydrogen or alkyl;
R⁸ is hydrogen, halo, alkyl, alkoxy or amino; and
R⁹ is hydrogen, halo, or alkyl.

The PRMT5 inhibitor may be identical to the compound described in PCT Publication No. WO2021066578A1, and the PRMT5 inhibitor of the present invention, which is a compound of Chemical Formula 1, or an optical isomer, a stereoisomer or an isotope variant thereof, or a pharmaceutically acceptable salt thereof, may be prepared in the same manner as the compound and the preparation method thereof described in WO2021066578A1.

In a specific example, the PRMT5 inhibitor is a compound of Chemical Formula 1, or an optical isomer, stereoisomer or isotope variant thereof, or a pharmaceutically acceptable salt thereof,
in the Chemical Formula 1,
X¹ and X² are independently CH or N;
when n is 0, Y is CH₂, O or NH; when n is 1, Y is CH or N;
when m is 0, Z is CH₂ or CH; when m is 1, Z is CH or C; or when m is 2, Z is C;
[symbol] is a single bond or a double bond.
R¹ is -D-R¹⁰, where D is a direct bond, —O—, —C(═O)—, —C≡C—, or —CR¹¹R¹²—; R¹⁰ is hydrogen, halo, hydroxy, cyano, C₁-C₇ alkyl, hydroxy-C₁-C₇ alkyl, halo-C₁-C₇ alkyl, halo-C₁-C₇ alkylsulfonate, di(C₁-C₇ alkyl)amino, C₁-C₇ alkylamino-C₁-C₇ alkyl, di(C₁-C₇ alkyl)amino-C₁-C₇ alkyl, di(C₁-C₇ alkyl)aminocarbonyl-C₁-C₇ alkyl, saturated or unsaturated C₃-C₁₀ carbocyclyl, saturated or unsaturated, 4- to 10-membered heterocyclyl, saturated or unsaturated C₃-C₁₀ carbocyclyl-C₁-C₇ alkyl, or saturated or unsaturated 4- to 10-membered heterocyclyl-alkyl; and
R¹¹ and R¹² are independently hydrogen, hydroxy or C₁-C₇ alkyl.

Specifically, R¹⁰ may be halo, C₁-C₇ alkyl, hydroxy-C₁-C₇ alkyl, halo-C₁-C₇ alkyl, saturated or unsaturated C₃-C₁₀ carbocyclyl, saturated or unsaturated 4- to 10-membered heterocyclyl, or saturated or unsaturated 4- to 10-membered heterocyclyl-alkyl.

The carbocycle or heterocycle may be substituted with 1 to 5 substituents selected from the group consisting of hydroxy, halo, oxo, formyl, nitrile, C₁-C₇ alkyl, C₁-C₇ alkoxy, hydroxy-C₁-C₇ alkyl, hydroxyhalo-C₁-C₇ alkyl, C₁-C₇ alkoxy-C₁-C₇ alkyl, halo-C₁-C₇ alkyl, nitrile-C₁-C₇ alkyl, C₁-C₇ alkylcarbonyl, C₁-C₇ alkylthiocarbonyl, C₁-C₇ alkoxycarbonyl, halo-C₁-C₇ alkylcarbonyl, saturated or unsaturated C₃-C₁₀ carbocyclyl, saturated or unsaturated C₃-C₁₀ carbocyclylcarbonyl, (C₁-C₇ alkyl)(halo-C₁-C₇ alkyl)amino, (C₁-C₇ alkyl) (saturated or unsaturated 4- to 10-membered heterocyclyl)amino, saturated or unsaturated 4- to 10-membered heterocyclyl, and saturated or unsaturated 4- to 10-membered heterocyclyl-C₁-C₇ alkyl.

Preferably, the carbocycle or heterocycle may be substituted with 1 to 5 substituents selected from the group consisting of hydroxy, halo, formyl, C₁-C₇ alkyl, hydroxy-C₁-C₇ alkyl, C₁-C₇ alkoxy-C₁-C₇ alkyl, halo-C₁-C₇ alkyl, C₁-C₇ alkylcarbonyl, C₁-C₇ alkoxycarbonyl, halo-C₁-C₇ alkylcarbonyl, saturated or unsaturated C₃-C₁₀ carbocyclyl, saturated or unsaturated C₃-C₁₀ carbocyclylcarbonyl, (C₁-C₇ alkyl)(halo-C₁-C₇ alkyl)amino, (C₁-C₇ alkyl)(saturated or unsaturated 4- to 10-membered heterocyclyl)amino, and saturated or unsaturated 4 to 10-membered heterocyclyl-C₁-C₇ alkyl.

The heterocycle may be substituted with one or two substituents selected from the group consisting of hydroxy, halo, formyl, C₁-C₇ alkyl, hydroxy-C₁-C₇ alkyl, C₁-C₇ alkoxy-C₁-C₇ alkyl, halo-C₁-C₇ alkyl, C₁-C₇ alkylcarbonyl, C₁-C₇ alkoxycarbonyl, halo-C₁-C₇ alkylcarbonyl, saturated or unsaturated C₃-C₁₀ carbocyclyl, saturated or unsaturated C₃-C₁₀ carbocyclylcarbonyl and saturated or unsaturated 4- to 10-membered heterocyclyl-C₁-C₇ alkyl; or the carbocycle may be substituted with one or two substituents selected from the group consisting of halo-C₁-C₇ alkyl, (C₁-C₇ alkyl)(halo-C₁-C₇ alkyl)amino, and (C₁-C₇ alkyl)(saturated or unsaturated 4- to 10-membered heterocyclyl)amino.

In a specific example, in the Chemical Formula 1, R¹ is -D-R¹⁰ and D is a direct bond; R¹⁰ may be hydrogen, halo, cyano, C₁-C₇ alkyl, halo-C₁-C₇ alkyl, di(C₁-C₇ alkyl)amino-C₁-C₇ alkyl, saturated or unsaturated 4- to 10-membered heterocyclyl, saturated or unsaturated C₃-C₁₀ carbocyclyl, or saturated or unsaturated 4- to 10-membered heterocyclyl-alkyl.

The heterocycle may be substituted with one or two substituents selected from the group consisting of hydroxy, halo, formyl, C₁-C₇ alkyl, C₁-C₇ alkoxy, hydroxy-C₁-C₇ alkyl, halo-C₁-C₇ alkyl, nitrile-C₁-C₇ alkyl, C₁-C₇ alkylcarbonyl, C₁-C₇ alkoxy-C₁-C₇ alkyl, C₁-C₇ alkoxycarbonyl, halo-C₁-C₇ alkylcarbonyl, and saturated or unsaturated 4- to 10-membered heterocyclyl;
R² is hydrogen or C₁-C₇ alkyl;
R³ may be hydrogen or C₁-C₇ alkyl;
R⁴, R⁵, R⁶ and R⁷ may be independently hydrogen or C₁-C₇ alkyl;
R⁸ may be hydrogen, halo, C₁-C₇ alkyl, C₁-C₇ alkoxy or amino; and
R⁹ may be hydrogen, halo or C₁-C₇ alkyl.

In the Chemical Formula 1, [symbol] may be a single bond.

The heterocycle is a saturated or unsaturated 4- to 8-membered hydrocarbon having one or two heteroatoms,
preferably, the heterocycle may be selected from the group consisting of tetrahydropyridine, dihydropyridine, piperidine, dihydropyran, tetrahydropyran, pyrrolidine, 2-oxa-6-azaspiroheptane, azetidine, morpholine, 3,3a,4,5,6,6a-hexahydro-1H-cyclopenta[c]pyrrole, oxazepane, 2-oxa-5-azabicyclo[2.2.1]heptane, pyridyl, tetrahydrofuran, 8-azabicyclo[3.2.1]octane, piperazine, 2-azabicyclo[3.3]heptane, 2-oxa-7-azabispiro[3.4]octane, 2-azabicyclo[2.2.1]heptane, 3-oxa-8-azabicyclo[3.2.1]octane, 3,4-dihydro-1H-pyrrolo[1,2-a]pyrazine, 3,4,6,7,8,8a-hexahydro-1H-pyrrolo[1,2]-a]pyrazine, pyrimidine, pyrazole, 2-oxa-7-azaspiro[3.5]nonane, and oxetane.

More preferably, the heterocycle may be selected from the group consisting of tetrahydropyridine, dihydropyridine, piperidine, tetrahydropyran, pyrrolidine, 2-oxa-6-azaspiroheptane, azetidine, morpholine, 3,3a,4,5,6,6a-hexahydro-1H-cyclopenta[c]pyrrole, pyridyl, 8-azabicyclo[3.2.1]octane, piperazine, 2-azaspiro[3.3]heptane, pyrazole and oxetane.

The carbocycle is selected from the group consisting of cyclohexane, cyclohexene, cyclopropane, cyclobutane and cyclopentane.
D is a direct bond, —O—, —C(═O)—, or —C≡C—;
R¹⁰ is halo, C₁-C₇ alkyl, hydroxy-C₁-C₇ alkyl, halo-C₁-C₇ alkyl, saturated or unsaturated C₃-C₁₀ carbocyclyl, saturated or unsaturated 4- to 10-membered heterocyclyl, or saturated or unsaturated 4- to 10-membered heterocyclyl-alkyl;
the carbocycle may be selected from the group consisting of hydroxy, halo, formyl, C₁-C₇ alkyl, hydroxy-C₁-C₇ alkyl, C₁-C₇ alkoxy-C₁-C₇ alkyl, halo-C₁-C₇ alkyl, C₁-C₇ alkylcarbonyl, C₁-C₇ alkoxycarbonyl, halo-C₁-C₇ alkylcarbonyl, saturated or unsaturated C₃-C₇ carbocyclyl, saturated or unsaturated C₃-C₇ carbocyclylcarbonyl, (C₁-C₇ alkyl)(halo-C₁-C₇ alkyl)amino, (C₁-C₇ alkyl)(saturated or unsaturated, 4- to 10-membered heterocyclyl)amino, and saturated or unsaturated 4- to 10-membered heterocyclyl-C₁-C₇ alkyl.

The heterocycle may be selected from the group consisting of tetrahydropyridine, dihydropyridine, piperidine, tetrahydropyran, pyrrolidine, 2-oxa-6-azaspiroheptane, azetidine, morpholine, 3,3a,4,5,6,6a-hexahydro-1H-cyclopenta[c]pyrrole, pyridyl, 8-azabicyclo[3.2.1]octane, piperazine, 2-azaspiro[3.3]heptane, pyrazole and oxetane.

The carbocycle may be selected from the group consisting of cyclohexane, cyclohexene and cyclopropane.

According to another embodiment disclosed herein, the PRMT5 inhibitor of Chemical Formula 1 in the pharmaceutical combination includes, but is not limited to, those listed in Table 1 below.

TABLE 1

| Number | Compound name |
|---|---|
| 1 | 4-[3-(3,4-dihydro-1H-isoquinolin-2-yl)-2-hydroxy-propyl]-8-methoxy-2,3- |
| | dihydro-1,4-benzoxazepin-5-one |
| 2 | 2-[3-(3,4-dihydro-1H-isoquinolin-2-yl)-2-hydroxy-propyl]-7-methoxy-4,5- |
| | dihydro-3H-2-benzazepin-1-one |
| 3 | 2-[3-(3,4-dihydro-1H-isoquinolin-2-yl)-2-hydroxy-propyl]-7-hydroxy-4,5- |
| | dihydro-3H-2-benzazepin-1-one |
| 4 | 2-[3-(3,4-dihydro-1H-isoquinolin-2-yl)-2-hydroxy-propyl]-7- |
| | (trifluoromethoxy)-4,5-dihydro-3H-2-benzazepin-1-one |
| 5 | 4-[(2R)-3-(3,4-dihydro-1H-isoquinolin-2-yl)-2-hydroxy-propyl]-8- |
| | (trifluoromethyl)-2,3-dihydro-1,4-benzoxazepin-5-one |
| 6 | 2-[(2R)-3-(3,4-dihydro-1H-isoquinolin-2-yl)-2-hydroxy-propyl]-7- |
| | (trifluoromethoxy)-4,5-dihydro-3H-2-benzazepin-1-one |
| 7 | 2-[(2R)-3-(3,4-dihydro-1H-isoquinolin-2-yl)-2-hydroxy-propyl]-7-ethoxy- |
| | 4,5-dihydro-3H-2-benzazepin-1-one |
| 8 | [2-[(2R)-3-(3,4-dihydro-1H-isoquinolin-2-yl)-2-hydroxy-propyl]-1-oxo-4,5- |
| | dihydro-3H-2-benzazepin-7-yl] trifluoromethanesulfonate |
| 9 | 2-[(2R)-3-(3,4-dihydro-1H-isoquinolin-2-yl)-2-hydroxy-propyl]-1-oxo-4,5- |
| | dihydro-3H-2-benzazepine-7-carbonitrile |
| 10 | 4-[(2R)-3-(3,4-dihydro-1H-isoquinolin-2-yl)-2-hydroxy-propyl]-8-methyl- |
| | 2,3-dihydro-1,4-benzoxazepin-5-one |
| 11 | 4-[(2R)-3-(3,4-dihydro-1H-isoquinolin-2-yl)-2-hydroxy-propyl]-8-propyl- |
| | 2,3-dihydro-1,4-benzoxazepin-5-one |
| 12 | 4-[(2R)-3-(3,4-dihydro-1H-isoquinolin-2-yl)-2-hydroxy-propyl]-8-isobutyl- |
| | 2,3-dihydro-1,4-benzoxazepin-5-one |
| 13 | 4-[(2R)-3-(3,4-dihydro-1H-isoquinolin-2-yl)-2-hydroxy-propyl]-8-(3,3,3- |
| | trifluoropropyl)-2,3-dihydro-1,4-benzoxazepin-5-one |
| 14 | tert-Butyl 4-[4-[(2R)-3-(3,4-dihydro-1H-isoquinolin-2-yl)-2-hydroxy- |
| | propyl]-5-oxo-2,3-dihydro-1,4-benzoxazepin-8-yl]-3,6-dihydro-2H-pyridine- |
| | 1-carboxylate |
| 15 | 4-[(2R)-3-(3,4-dihydro-1H-isoquinolin-2-yl)-2-hydroxy-propyl]-8-(1,2,3,6- |
| | tetrahydropyridin-4-yl)-2,3-dihydro-1,4-benzoxazepin-5-one |
| 16 | 8-(1-Acetyl-3,6-dihydro-2H-pyridin-4-yl)-4-[(2R)-3-(3,4-dihydro-1H- |
| | isoquinolin-2-yl)-2-hydroxy-propyl]-2,3-dihydro-1,4-benzoxazepin-5-one |
| 17 | 8-(1-Acetyl-4-piperidyl)-4-[(2R)-3-(3,4-dihydro-1H-isoquinolin-2-yl)-2- |
| | hydroxy-propyl]-2,3-dihydro-1,4-benzoxazepin-5-one |
| 18 | 4-[(2R)-3-(3,4-dihydro-1H-isoquinolin-2-yl)-2-hydroxy-propyl]-8-(1-ethyl- |
| | 3,6-dihydro-2H-pyridin-4-yl)-2,3-dihydro-1,4-benzoxazepin-5-one |
| 19 | 4-[(2R)-3-(3,4-dihydro-1H-isoquinolin-2-yl)-2-hydroxy-propyl]-8-(1- |
| | isopropyl-3,6-dihydro-2H-pyridin-4-yl)-2,3-dihydro-1,4-benzoxazepin-5-one |
| 20 | 4-[(2R)-3-(3,4-dihydro-1H-isoquinolin-2-yl)-2-hydroxy-propyl]-8-(3,6- |
| | dihydro-2H-pyran-4-yl)-2,3-dihydro-1,4-benzoxazepin-5-one |
| 21 | 4-[(2R)-3-(3,4-dihydro-1H-isoquinolin-2-yl)-2-hydroxy-propyl]-8- |
| | tetrahydropyran-4-yl-2,3-dihydro-1,4-benzoxazepin-5-one |
| 22 | 4-[(2R)-3-(3,4-dihydro-1H-isoquinolin-2-yl)-2-hydroxy-propyl]-8- |
| | (morpholinomethyl)-2,3-dihydro-1,4-benzoxazepin-5-one |
| 23 | 4-[(2R)-3-(3,4-dihydro-1H-isoquinolin-2-yl)-2-hydroxy-propyl]-8-(1- |
| | piperidylmethyl)-2,3-dihydro-1,4-benzoxazepin-5-one |
| 24 | 4-[(2R)-3-(3,4-dihydro-1H-isoquinolin-2-yl)-2-hydroxy-propyl]-8- |
| | (pyrrolidin-1-ylmethyl)-2,3-dihydro-1,4-benzoxazepin-5-one |
| 25 | 4-[(2R)-3-(3,4-dihydro-1H-isoquinolin-2-yl)-2-hydroxy-propyl]-8- |
| | [(dimethylamino)methyl]-2,3-dihydro-1,4-benzoxazepin-5-one |
| 26 | 8-(Diethylaminomethyl)-4-[(2R)-3-(3,4-dihydro-1H-isoquinolin-2-yl)-2- |
| | hydroxy-propyl]-2,3-dihydro-1,4-benzoxazine phen-5-one |
| 27 | 4-[(2R)-3-(3,4-dihydro-1H-isoquinolin-2-yl)-2-hydroxy-propyl]-8-[(4- |
| | methyl-1-piperidyl)methyl]-2,3-dihydro-1,4-benzoxazepin-5-one |
| 28 | 4-[(2R)-3-(3,4-dihydro-1H-isoquinolin-2-yl)-2-hydroxy-propyl]-8-(2-oxa-6- |
| | azaspiro[3.3]heptan-6-ylmethyl)-2,3-dihydro-1,4-benzoxazepin-5-one |
| 29 | 4-[(2R)-3-(3,4-dihydro-1H-isoquinolin-2-yl)-2-hydroxy-propyl]-8-[(3- |
| | methoxyazetidin-1-yl)methyl]-2,3-dihydro-1,4-benzoxazepin-5-one |
| 30 | 4-[(2R)-3-(3,4-dihydro-1H-isoquinolin-2-yl)-2-hydroxy-propyl]-8-[(2- |
| | methylmorpholin-4-yl)methyl]-2,3-dihydro-1,4-benzoxazepin-5-one |
| 31 | 8-[(4,4-difluoro-1-piperidyl)methyl]-4-[(2R)-3-(3,4-dihydro-1H-isoquinolin- |
| | 2-yl)-2-hydroxy-propyl]-2,3-dihydro-1,4-benzoxazepin-5-one |
| 32 | 4-[(2R)-3-(3,4-dihydro-1H-isoquinolin-2-yl)-2-hydroxy-propyl]-8-[(4- |
| | fluoro-1-piperidyl)methyl]-2,3-dihydro-1,4-benzoxazepin-5-one |
| 33 | 4-[(2R)-3-(3,4-dihydro-1H-isoquinolin-2-yl)-2-hydroxy-propyl]-8-[(3,5- |
| | dimethyl-1-piperidyl)methyl]-2,3-dihydro-1,4-benzoxazepin-5-one |
| 34 | 8-(3,3a,4,5,6,6a-Hexahydro-1H-cyclopenta[c]pyrrol-2-ylmethyl)-4-[(2R)-3- |
| | (3,4-dihydro-1H-isoquinolin-2-yl)-2-hydroxy-propyl]-2,3-dihydro-1,4- |
| | benzoxazepin-5-one |
| 35 | 4-[(2R)-3-(3,4-dihydro-1H-isoquinolin-2-yl)-2-hydroxy-propyl]-8-[(3- |
| | hydroxypyrrolidin-1-yl)methyl]-2,3-dihydro-1,4-benzoxazepin-5-one |
| 36 | 4-[(2R)-3-(3,4-dihydro-1H-isoquinolin-2-yl)-2-hydroxy-propyl]-8-[[(2R)-2- |
| | methylpyrrolidin-1-yl]methyl]-2,3-dihydro-1,4-benzoxazepin-5-one |
| 37 | 4-[(2R)-3-(3,4-dihydro-1H-isoquinolin-2-yl)-2-hydroxy-propyl]-8-[[3- |
| | (hydroxymethyl)-1-piperidyl]methyl]-2,3-dihydro-1,4-benzoxazepin-5-one |
| 38 | 4-[(2R)-3-(3,4-dihydro-1H-isoquinolin-2-yl)-2-hydroxy-propyl]-8-[(4- |
| | methoxy-1-piperidyl)methyl]-2,3-dihydro-1,4-benzoxazepin-5-one |
| 39 | 4-[(2R)-3-(3,4-dihydro-1H-isoquinolin-2-yl)-2-hydroxy-propyl]-8-(1,4- |
| | oxazepan-4-ylmethyl)-2,3-dihydro-1,4-benzoxazepin-5-one |
| 40 | 4-[(2R)-3-(3,4-dihydro-1H-isoquinolin-2-yl)-2-hydroxy-propyl]-8-[(3- |
| | hydroxy-3-methyl-pyrrolidin-1-yl)methyl]-2,3-dihydro-1,4-benzoxazepin-5- |
| | one |
| 41 | 4-[(2R)-3-(3,4-dihydro-1H-isoquinolin-2-yl)-2-hydroxy-propyl]-8-[[3- |
| | (hydroxymethyl)morpholin-4-yl]methyl]-2,3-dihydro-1,4-benzoxazepin-5- |
| | one |
| 42 | 8-[(3,3-difluoro-1-piperidyl)methyl]-4-[(2R)-3-(3,4-dihydro-1H-isoquinolin- |
| | 2-yl)-2-hydroxy-propyl]-2,3-dihydro-1,4-benzoxazepin-5-one |
| 43 | 4-[(2R)-3-(3,4-dihydro-1H-isoquinolin-2-yl)-2-hydroxy-propyl]-8-[(3- |
| | fluoro-1-piperidyl)methyl]-2,3-dihydro-1,4-benzoxazepin-5-one |
| 44 | 4-[(2R)-3-(3,4-dihydro-1H-isoquinolin-2-yl)-2-hydroxy-propyl]-8-[(3- |
| | hydroxy-1-piperidyl)methyl]-2,3-dihydro-1,4-benzoxazepin-5-one |
| 45 | 4-[(2R)-3-(3,4-dihydro-1H-isoquinolin-2-yl)-2-hydroxy-propyl]-8-[(4- |
| | hydroxy-1-piperidyl)methyl]-2,3-dihydro-1,4-benzoxazepin-5-one |
| 46 | 4-[(2R)-3-(3,4-dihydro-1H-isoquinolin-2-yl)-2-hydroxy-propyl]-8-[[(2S)-2- |
| | (hydroxymethyl)pyrrolidin-1-yl]methyl]-2,3-dihydro-1,4-benzoxazepin-5- |
| | one |
| 47 | 4-[(2R)-3-(3,4-dihydro-1H-isoquinolin-2-yl)-2-hydroxy-propyl]-8-[[(2R)-2- |
| | (hydroxymethyl)pyrrolidin-1-yl]methyl]-2,3-dihydro-1,4-benzoxazepin-5- |
| | one |
| 48 | 4-[(2R)-3-(3,4-dihydro-1H-isoquinolin-2-yl)-2-hydroxy-propyl]-8-[(2- |
| | ethylpyrrolidin-1-yl)methyl]-2,3-dihydro-1,4-benzoxazepin-5-one |
| 49 | 4-[(2R)-3-(3,4-dihydro-1H-isoquinolin-2-yl)-2-hydroxy-propyl]-8-[[(2S)-2- |
| | (methoxymethyl)pyrrolidin-1-yl]methyl]-2,3-dihydro-1,4-benzoxazepin-5- |
| | one |
| 50 | 4-[(2R)-3-(3,4-dihydro-1H-isoquinolin-2-yl)-2-hydroxy-propyl]-8-[[(2R)-2- |
| | (methoxymethyl)pyrrolidin-1-yl]methyl]-2,3-dihydro-1,4-benzoxazepin-5- |
| | one |
| 51 | 4-[(2R)-3-(3,4-dihydro-1H-isoquinolin-2-yl)-2-hydroxy-propyl]-8-[(3- |
| | fluoropyrrolidin-1-yl)methyl]-2,3-dihydro-1,4-benzoxazepin-5-one |
| 52 | 8-[(3,3-difluoropyrrolidin-1-yl)methyl]-4-[(2R)-3-(3,4-dihydro-1H- |
| | isoquinolin-2-yl)-2-hydroxy-propyl]-2,3-dihydro-1,4-benzoxazepin-5-one |
| 53 | 8-[(4-acetylpiperazin-1-yl)methyl]-4-[(2R)-3-(3,4-dihydro-1H-isoquinolin-2- |
| | yl)-2-hydroxy-propyl]-2,3-dihydro-1,4-benzoxazepin-5-one |
| 54 | 4-[(2R)-3-(3,4-dihydro-1H-isoquinolin-2-yl)-2-hydroxy-propyl]-8-[(4- |
| | methylpiperazin-1-yl)methyl]-2,3-dihydro-1,4-benzoxazepin-5-one |
| 55 | 4-[(2R)-3-(3,4-dihydro-1H-isoquinolin-2-yl)-2-hydroxy-propyl]-8-(2-oxa-5- |
| | azabicyclo[2.2.1]heptan-5-ylmethyl)-2,3-dihydro-1,4-benzoxazepin-5-one |
| 56 | 4-[(2R)-3-(3,4-dihydro-1H-isoquinolin-2-yl)-2-hydroxy-propyl]-8-(3- |
| | pyridylmethoxy)-2,3-dihydro-1,4-benzoxazepin-5-one |
| 57 | 4-[(2R)-3-(3,4-dihydro-1H-isoquinolin-2-yl)-2-hydroxy-propyl]-8-(4- |
| | pyridylmethoxy)-2,3-dihydro-1,4-benzoxazepin-5-one |
| 58 | 4-[(2R)-3-(3,4-dihydro-1H-isoquinolin-2-yl)-2-hydroxy-propyl]-8-[(2- |
| | fluoro-4-pyridyl)methoxy]-2,3-dihydro-1,4-benzoxazepin-5-one |
| 59 | 8-[(2,6-Dichloro-4-pyridyl)methoxy]-4-[(2R)-3-(3,4-dihydro-1H- |
| | isoquinolin-2-yl)-2-hydroxy-propyl]-2,3-dihydro-1,4-benzoxazepin-5-one |
| 60 | 8-[(2,3-difluoro-4-pyridyl)methoxy]-4-[(2R)-3-(3,4-dihydro-1H-isoquinolin- |
| | 2-yl)-2-hydroxy-propyl]-2,3-dihydro-1,4-benzoxazepin-5-one |
| 61 | 4-[(2R)-3-(3,4-dihydro-1H-isoquinolin-2-yl)-2-hydroxy-propyl]-8-[(6- |
| | fluoro-3-pyridyl)methoxy]-2,3-dihydro-1,4-benzoxazepin-5-one |
| 62 | 4-[(2R)-3-(3,4-dihydro-1H-isoquinolin-2-yl)-2-hydroxy-propyl]-8-[(3- |
| | fluoro-4-pyridyl)methoxy]-2,3-dihydro-1,4-benzoxazepin-5-one |
| 63 | 4-[(2R)-3-(3,4-dihydro-1H-isoquinolin-2-yl)-2-hydroxy-propyl]-8-[(5- |
| | fluoro-2-pyridyl)methoxy]-2,3-dihydro-1,4-benzoxazepin-5-one |
| 64 | 4-[(2R)-3-(3,4-dihydro-1H-isoquinolin-2-yl)-2-hydroxy-propyl]-8- |
| | tetrahydropyran-4-yloxy-2,3-dihydro-1,4-benzoxazepin-5-one |
| 65 | 4-[(2R)-3-(3,4-dihydro-1H-isoquinolin-2-yl)-2-hydroxy-propyl]-8- |
| | (tetrahydropyran-2-ylmethoxy)-2,3-dihydro-1,4-benzoxazepin-5-one |
| 66 | 8-(Cyclohexylmethoxy)-4-[(2R)-3-(3,4-dihydro-1H-isoquinolin-2-yl)-2- |
| | hydroxy-propyl]-2,3-dihydro-1,4-benzoxazepin-5-one |
| 67 | 4-[(2R)-3-(3,4-dihydro-1H-isoquinolin-2-yl)-2-hydroxy-propyl]-8- |
| | (tetrahydrofuran-2-ylmethoxy)-2,3-dihydro-1,4-benzoxazepin-5-one |
| 68 | 4-[(2R)-3-(3,4-dihydro-1H-isoquinolin-2-yl)-2-hydroxy-propyl]-8-[(1- |
| | methyl-3-piperidyl)oxy]-2,3-dihydro-1,4-benzoxazepin-5-one |
| 69 | 4-[(2R)-3-(3,4-dihydro-1H-isoquinolin-2-yl)-2-hydroxy-propyl]-8-[(1-ethyl- |
| | 3-piperidyl)oxy]-2,3-dihydro-1,4-benzoxazepin-5-one |
| 70 | 8-[(1-acetyl-4-piperidyl)methoxy]-4-[(2R)-3-(3,4-dihydro-1H-isoquinolin-2- |
| | yl)-2-hydroxy-propyl]-2,3-dihydro-1,4-benzoxazepin-5-one |
| 71 | 4-[(2R)-3-(3,4-dihydro-1H-isoquinolin-2-yl)-2-hydroxy-propyl]-8-(2,2,2- |
| | trifluoroethoxy)-2,3-dihydro-1,4-benzoxazepin-5-one |
| 72 | 4-[(2R)-3-(3,4-dihydro-1H-isoquinolin-2-yl)-2-hydroxy-propyl]-8-[2- |
| | (dimethylamino)ethoxy]-2,3-dihydro-1,4-benzoxazepin-5-one |
| 73 | 4-[(2R)-3-(3,4-dihydro-1H-isoquinolin-2-yl)-2-hydroxy-propyl]-8-(2- |
| | morpholinoethoxy)-2,3-dihydro-1,4-benzoxazepin-5-one |
| 74 | 4-[(2R)-3-(3,4-dihydro-1H-isoquinolin-2-yl)-2-hydroxy-propyl]-8-[2-(2- |
| | oxopyrrolidin-1-yl)ethoxy]-2,3-dihydro-1,4-benzoxazepin-5-one |
| 75 | 4-[(2R)-3-(3,4-dihydro-1H-isoquinolin-2-yl)-2-hydroxy-propyl]-8-(2- |
| | pyridyloxy)-2,3-dihydro-1,4-benzoxazepin-5-one |
| 76 | 4-[(2R)-3-(3,4-dihydro-1H-isoquinolin-2-yl)-2-hydroxy-propyl]-8-[(6-oxo-3- |
| | piperidyl)oxy]-2,3-dihydro-1,4-benzoxazepin-5-one |
| 77 | tert-Butyl 4-[[4-[(2R)-3-(3,4-dihydro-1H-isoquinolin-2-yl)-2-hydroxy- |
| | propyl]-5-oxo-2,3-dihydro-1,4-benzoxazepin-8-yl]oxy]piperidine-1- |
| | carboxylate |
| 78 | 8-[(1-acetyl-4-piperidyl)oxy]-4-[(2R)-3-(3,4-dihydro-1H-isoquinolin-2-yl)-2- |
| | hydroxy-propyl]-2,3-dihydro-1,4-benzoxazepin-5-one |
| 79 | 8-[(1-acetyl-3-piperidyl)oxy]-4-[(2R)-3-(3,4-dihydro-1H-isoquinolin-2-yl)-2- |
| | hydroxy-propyl]-2,3-dihydro-1,4-benzoxazepin-5-one |
| 80 | 8-(1-acetylpyrrolidin-3-yl)oxy-4-[(2R)-3-(3,4-dihydro-1H-isoquinolin-2-yl)- |
| | 2-hydroxy-propyl]-2,3-dihydro-1,4-benzoxazepin-5-one |
| 81 | 8-[(3R)-1-acetylpyrrolidin-3-yl]oxy-4-[(2R)-3-(3,4-dihydro-1H-isoquinolin- |
| | 2-yl)-2-hydroxy-propyl]-2,3-dihydro-1,4-benzoxazepin-5-one |
| 82 | 8-[(3S)-1-acetylpyrrolidin-3-yl]oxy-4-[(2R)-3-(3,4-dihydro-1H-isoquinolin- |
| | 2-yl)-2-hydroxy-propyl]-2,3-dihydro-1,4-benzoxazepin-5-one |
| 83 | 8-(1-acetylacetidin-3-yl)oxy-4-[(2R)-3-(3,4-dihydro-1H-isoquinolin-2-yl)-2- |
| | hydroxy-propyl]-2,3-dihydro-1,4-benzoxazepin-5-one |
| 84 | 4-[(2R)-3-(3,4-dihydro-1H-isoquinolin-2-yl)-2-hydroxy-propyl]-8-(1- |
| | propanoylazetidin-3-yl)oxy-2,3-dihydro-1,4-benzoxazepin-5-one |
| 85 | 8-[1-(cyclopropanecarbonyl)azetidin-3-yl]oxy-4-[(2R)-3-(3,4-dihydro-1H- |
| | isoquinolin-2-yl)-2-hydroxy-propyl]-2,3-dihydro-1,4-benzoxazepin-5-one |
| 86 | Methyl 3-[[4-[(2R)-3-(3,4-dihydro-1H-isoquinolin-2-yl)-2-hydroxy-propyl]- |
| | 5-oxo-2,3-dihydro-1,4-benzoxazepin-8-yl]oxy]azetidine-1-carboxylate |
| 87 | 4-[(2R)-3-(3,4-dihydro-1H-isoquinolin-2-yl)-2-hydroxy-propyl]-8-[(1- |
| | methyl-4-piperidyl)oxy]-2,3-dihydro-1,4-benzoxazepin-5-one |
| 88 | 4-[(2R)-3-(3,4-dihydro-1H-isoquinolin-2-yl)-2-hydroxy-propyl]-8-[(1-ethyl- |
| | 4-piperidyl)oxy]-2,3-dihydro-1,4-benzoxazepin-5-one |
| 89 | 4-[(2R)-3-(3,4-dihydro-1H-isoquinolin-2-yl)-2-hydroxy-propyl]-8-[(1- |
| | isopropyl-4-piperidyl)oxy]-2,3-dihydro-1,4-benzoxazepin-5-one |
| 90 | 8-[(1-Cyclopropyl-4-piperidyl)oxy]-4-[(2R)-3-(3,4-dihydro-1H-isoquinolin- |
| | 2-yl)-2-hydroxy-propyl]-2,3-dihydro-1,4-benzoxazepin-5-one |
| 91 | 8-[(1-Cyclobutyl-4-piperidyl)oxy]-4-[(2R)-3-(3,4-dihydro-1H-isoquinolin-2- |
| | yl)-2-hydroxy-propyl]-2,3-dihydro-1,4-benzoxazepin-5-one |
| 92 | 4-[(2R)-3-(3,4-dihydro-1H-isoquinolin-2-yl)-2-hydroxy-propyl]-8-[[1- |
| | (oxetan-3-yl)-4-piperidyl]oxy]-2,3-dihydro-1,4-benzoxazepin-5-one |
| 93 | 4-[(2R)-3-(3,4-dihydro-1H-isoquinolin-2-yl)-2-hydroxy-propyl]-8-(1- |
| | methylazetidin-3-yl)oxy-2,3-dihydro-1,4-benzoxazepin-5-one |
| 94 | 4-[(2R)-3-(3,4-dihydro-1H-isoquinolin-2-yl)-2-hydroxy-propyl]-8-(1- |
| | ethylazetidin-3-yl)oxy-2,3-dihydro-1,4-benzoxazepin-5-one |
| 95 | 4-[(2R)-3-(3,4-dihydro-1H-isoquinolin-2-yl)-2-hydroxy-propyl]-8-(1- |
| | isopropylazetidin-3-yl)oxy-2,3-dihydro-1,4-benzoxazepin-5-one |
| 96 | 4-[(2R)-3-(3,4-dihydro-1H-isoquinolin-2-yl)-2-hydroxy-propyl]-8-[1- |
| | (oxetan-3-yl)azetidin-3-yl]oxy-2,3-dihydro-1,4-benzoxazepin-5-one |
| 97 | 4-[(2R)-3-(3,4-dihydro-1H-isoquinolin-2-yl)-2-hydroxy-propyl]-8-[(1- |
| | ethylazetidin-3-yl)methoxy]-2,3-dihydro-1,4-benzoxazepin-5-one |
| 98 | 4-[(2R)-3-(3,4-dihydro-1H-isoquinolin-2-yl)-2-hydroxy-propyl]-8-(1- |
| | methylpyrrolidin-3-yl)oxy-2,3-dihydro-1,4-benzoxazepin-5-one |
| 99 | 4-[(2R)-3-(3,4-dihydro-1H-isoquinolin-2-yl)-2-hydroxy-propyl]-8-[(3R)-1- |
| | ethylpyrrolidin-3-yl]oxy-2,3-dihydro-1,4-benzoxazepin-5-one |
| 100 | 4-[(2R)-3-(3,4-dihydro-1H-isoquinolin-2-yl)-2-hydroxy-propyl]-8-(2- |
| | pyridyloxy)-2,3-dihydro-1,4-benzoxazepin-5-one |
| 101 | 4-[(2R)-3-(3,4-dihydro-1H-isoquinolin-2-yl)-2-hydroxy-propyl]-8-[[(2S)-1- |
| | isopropylpyrrolidin-2-yl]methoxy]-2,3-dihydro-1,4-benzoxazepin-5-one |
| 102 | 4-[(2R)-3-(3,4-dihydro-1H-isoquinolin-2-yl)-2-hydroxy-propyl]-8-[(1- |
| | methyl-4-piperidyl)methoxy]-2,3-dihydro-1,4-benzoxazepin-5-one |
| 103 | 4-[(2R)-3-(3,4-dihydro-1H-isoquinolin-2-yl)-2-hydroxy-propyl]-8-[(1-ethyl- |
| | 4-piperidyl)methoxy]-2,3-dihydro-1,4-benzoxazepin-5-one |
| 104 | 4-[(2R)-3-(3,4-dihydro-1H-isoquinolin-2-yl)-2-hydroxy-propyl]-8-[(4- |
| | methylmorpholin-2-yl)methoxy]-2,3-dihydro-1,4-benzoxazepin-5-one |
| 105 | 4-[(2R)-3-(3,4-dihydro-1H-isoquinolin-2-yl)-2-hydroxy-propyl]-8-[(4- |
| | ethylmorpholin-2-yl)methoxy]-2,3-dihydro-1,4-benzoxazepin-5-one |
| 106 | 4-[(2R)-3-(3,4-dihydro-1H-isoquinolin-2-yl)-2-hydroxy-propyl]-8-[(1- |
| | methylpyrrolidin-3-yl)methoxy]-2,3-dihydro-1,4-benzoxazepin-5-one |
| 107 | 4-[(2R)-3-(3,4-dihydro-1H-isoquinolin-2-yl)-2-hydroxy-propyl]-8-[(1- |
| | methyl-3-piperidyl)methoxy]-2,3-dihydro-1,4-benzoxazepin-5-one |
| 108 | 4-[(2R)-3-(3,4-dihydro-1H-isoquinolin-2-yl)-2-hydroxy-propyl]-6-fluoro-8- |
| | (morpholinomethyl)-2,3-dihydro-1,4-benzoxazepin-5-one |
| 109 | 4-[(2R)-3-(3,4-dihydro-1H-isoquinolin-2-yl)-2-hydroxy-propyl]-6-fluoro-8- |
| | [(1-methyl-4-piperidyl)oxy]-2,3-dihydro-1,4-benzoxazepin-5-one |
| 110 | 4-[(2R)-3-(3,4-dihydro-1H-isoquinolin-2-yl)-2-hydroxy-propyl]-8-[(1-ethyl- |
| | 4-piperidyl)oxy]-2,2-dimethyl-3H-1,4-benzoxazepin-5-one |
| 111 | 4-[(2R)-3-(3,4-dihydro-1H-isoquinolin-2-yl)-2-hydroxy-propyl]-2,2- |
| | dimethyl-8-(morpholinomethyl)-3H-1,4-benzoxazepin-5-one |
| 112 | 4-[(2R)-3-(3,4-dihydro-1H-isoquinolin-2-yl)-2-hydroxy-propyl]-2,2- |
| | dimethyl-8-[(1-methyl-4-piperidyl)oxy]-3H-1,4-benzoxazepin-5-one |
| 113 | 4-[(2R)-3-(3,4-dihydro-1H-isoquinolin-2-yl)-2-hydroxy-propyl]-2,2- |
| | dimethyl-8-(4-pyridylmethoxy)-3H-1,4-benzoxazepin-5-one |
| 114 | 8-(1-acetylasetidin-3-yl)oxy-4-[(2R)-3-(3,4-dihydro-1H-isoquinolin-2-yl)-2- |
| | hydroxy-propyl]-2,2-dimethyl-3H-1,4-benzoxazepin-5-one |
| 115 | 4-[(2R)-3-(3,4-dihydro-1H-isoquinolin-2-yl)-2-hydroxy-propyl]-1-methyl-8- |
| | (morpholinomethyl)-2,3-dihydro-1,4-benzodiazepin-5-one |
| 116 | 4-[(2R)-3-(3,4-dihydro-1H-isoquinolin-2-yl)-2-hydroxy-propyl]-1-methyl-8- |
| | (3,3,3-trifluoropropyl)-2,3-dihydro-1,4-benzodiazepin-5-one |
| 117 | 8-(Cyclohexylmethyl)-4-[(2R)-3-(3,4-dihydro-1H-isoquinolin-2-yl)-2- |
| | hydroxy-propyl]-1-methyl-2,3-dihydro-1,4-benzodiazepin-5-one |
| 118 | 8-(Cyclohexen-1-yl)-4-[(2R)-3-(3,4-dihydro-1H-isoquinolin-2-yl)-2- |
| | hydroxy-propyl]-1-methyl-2,3-dihydro-1,4-benzodiazepin-5-one |
| 119 | 4-[(2R)-3-(3,4-dihydro-1H-isoquinolin-2-yl)-2-hydroxy-propyl]-1-methyl-8- |
| | [4-(trifluoromethyl)cyclohexen-1-yl]-2,3-dihydro-1,4-benzodiazepin-5-one |
| 120 | tert-Butyl 4-[4-[(2R)-3-(3,4-dihydro-1H-isoquinolin-2-yl)-2-hydroxy- |
| | propyl]-1-methyl-5-oxo-2,3-dihydro-1,4-benzodiazepin-8-yl]-3,6-dihydro- |
| | 2H-pyridine-1-carboxylate |
| 121 | 8-[(1-acetyl-4-piperidyl)oxy]-4-[(2R)-3-(3,4-dihydro-1H-isoquinolin-2-yl)-2- |
| | hydroxy-propyl]-1-methyl-2,3-dihydro-1,4-benzodiazepin-5-one |
| 122 | 8-(1-acetylacetidin-3-yl)oxy-4-[(2R)-3-(3,4-dihydro-1H-isoquinolin-2-yl)-2- |
| | hydroxy-propyl]-1-methyl-2,3-dihydro-1,4-benzodiazepin-5-one |
| 123 | 8-[(3R)-1-acetylpyrrolidin-3-yl]oxy-4-[(2R)-3-(3,4-dihydro-1H-isoquinolin- |
| | 2-yl)-2-hydroxy-propyl]-1-methyl-2,3-dihydro-1,4-benzodiazepin-5-one |
| 124 | 8-[(3S)-1-acetylpyrrolidin-3-yl]oxy-4-[(2R)-3-(3,4-dihydro-1H-isoquinolin- |
| | 2-yl)-2-hydroxy-propyl]-1-methyl-2,3-dihydro-1,4-benzodiazepin-5-one |
| 125 | 4-[(2R)-3-(3,4-dihydro-1H-isoquinolin-2-yl)-2-hydroxy-propyl]-1-methyl-8- |
| | [(1-methyl-4-piperidyl)oxy]-2,3-dihydro-1,4-benzodiazepin-5-one |
| 126 | 4-[(2R)-3-(3,4-dihydro-1H-isoquinolin-2-yl)-2-hydroxy-propyl]-8-[(1-ethyl- |
| | 4-piperidyl)oxy]-1-methyl-2,3-dihydro-1,4-benzodiazepin-5-one |
| 127 | 8-[(1-Cyclobutyl-4-piperidyl)oxy]-4-[(2R)-3-(3,4-dihydro-1H-isoquinolin-2- |
| | yl)-2-hydroxy-propyl]-1-methyl-2,3-dihydro-1,4-benzodiazepin-5-one |
| 128 | 4-[(2R)-3-(3,4-dihydro-1H-isoquinolin-2-yl)-2-hydroxy-propyl]-1-methyl-8- |
| | [(1-tetrahydrofuran-3-yl-4-piperidyl)oxy]-2,3-dihydro-1,4-benzodiazepin-5- |
| | one |
| 129 | 4-[(2R)-3-(3,4-dihydro-1H-isoquinolin-2-yl)-2-hydroxy-propyl]-1-methyl-8- |
| | [[1-(oxetan-3-yl)-4-piperidyl]oxy]-2,3-dihydro-1,4-benzodiazepin-5-one |
| 130 | 4-[(2R)-3-(3,4-dihydro-1H-isoquinolin-2-yl)-2-hydroxy-propyl]-1-methyl-8- |
| | [(1-methyl-3-piperidyl)oxy]-2,3-dihydro-1,4-benzodiazepin-5-one |
| 131 | 4-[(2R)-3-(3,4-dihydro-1H-isoquinolin-2-yl)-2-hydroxy-propyl]-1-methyl-8- |
| | [(1-methylpyrrolidin-3-yl)methoxy]-2,3-dihydro-1,4-benzodiazepin-5-one |
| 132 | 4-[(2R)-3-(3,4-dihydro-1H-isoquinolin-2-yl)-2-hydroxy-propyl]-1-methyl-8- |
| | (1-methylpyrrolidin-3-yl)oxy-2,3-dihydro-1,4-benzodiazepin-5-one |
| 133 | 4-[(2R)-3-(3,4-dihydro-1H-isoquinolin-2-yl)-2-hydroxy-propyl]-1-methyl-8- |
| | [(1-methyl-3-piperidyl)methoxy]-2,3-dihydro-1,4-benzodiazepin-5-one |
| 134 | 4-[(2R)-3-(3,4-dihydro-1H-isoquinolin-2-yl)-2-hydroxy-propyl]-8-[(3- |
| | fluoro-4-pyridyl)methoxy]-1-methyl-2,3-dihydro-1,4-benzodiazepin-5-one |
| 135 | 4-[(2R)-3-(3,4-dihydro-1H-isoquinolin-2-yl)-2-hydroxy-propyl]-8-[(3- |
| | fluoro-4-piperidyl)oxy]-1-methyl-2,3-dihydro-1,4-benzodiazepin-5-one 2 |
| | hydrochloride |
| 136 | 4-[(2R)-3-(3,4-dihydro-1H-isoquinolin-2-yl)-2-hydroxy-propyl]-8-[(1-ethyl- |
| | 3-fluoro-4-piperidyl)oxy]-1-methyl-2,3-dihydro-1,4-benzodiazepin-5-one |
| 137 | 4-[(2R)-3-(3,4-dihydro-1H-isoquinolin-2-yl)-2-hydroxy-propyl]-8-[(8-ethyl- |
| | 8-azabicyclo[3.2.1]octan-3-yl)oxy]-1-methyl-2,3-dihydro-1,4- |
| | benzodiazepin-5-one |
| 138 | 4-[(2R)-3-(3,4-dihydro-1H-isoquinolin-2-yl)-2-hydroxy-propyl]-1-methyl-8- |
| | [(2-methyl-4-piperidyl)oxy]-2,3-dihydro-1,4-benzodiazepin-5-one 2 |
| | hydrochloride |
| 139 | 4-[(2R)-3-(3,4-dihydro-1H-isoquinolin-2-yl)-2-hydroxy-propyl]-8-[(5- |
| | fluoro-2-pyridyl)methoxy]-1-methyl-2,3-dihydro-1,4-benzodiazepin-5-one |
| 140 | 4-[(2R)-3-(3,4-dihydro-1H-isoquinolin-2-yl)-2-hydroxypropyl]-2-methyl-8- |
| | (4-piperidyloxy)-2,3-dihydro-1,4-benzoxazepin-5-one 2 hydrochloride |
| 141 | 4-[(2R)-3-(3,4-dihydro-1H-isoquinolin-2-yl)-2-hydroxypropyl]-2-methyl-8- |
| | [(1-methyl-4-piperidyl)oxy]-2,3-dihydro-1,4-benzoxazepin-5-one |
| 142 | (2R)-8-(1-acetylasetidin-3-yl)oxy-4-[(2R)-3-(3,4-dihydro-1H-isoquinolin-2- |
| | yl)-2-hydroxypropyl]-2-methyl-2,3-dihydro-1,4-benzoxazepin-5-one |
| 143 | (2R)-4-[(2R)-3-(3,4-dihydro-1H-isoquinolin-2-yl)-2-hydroxypropyl]-2- |
| | methyl-8-[(1-methyl-4-piperidyl)oxy]-2,3-dihydro-1,4-benzoxazepin-5-one |
| 144 | 4-[(2R)-3-(3,4-dihydro-1H-isoquinolin-2-yl)-2-hydroxypropyl]-2-methyl-8- |
| | [(1-methyl-4-piperidyl)oxy]-2,3-dihydropyrido[3,2-f][1,4]oxazepin-5-one |
| 145 | 4-[(2R)-3-(3,4-dihydro-1H-isoquinolin-2-yl)-2-hydroxypropyl]-2,2- |
| | dimethyl-8-[(1-methyl-4-piperidyl)oxy]-3H-pyrido[3,2-f][1,4]oxazepin-5- |
| | one |
| 146 | (2S)-4-[(2R)-3-(3,4-dihydro-1H-isoquinolin-2-yl)-2-hydroxypropyl]-2- |
| | methyl-8-[(1-methyl-4-piperidyl)oxy]-2,3-dihydro-1,4-benzoxazepin-5-one |
| 147 | 4-[3-(3,4-dihydro-1H-isoquinolin-2-yl)-2-hydroxy-propyl]-2,3-dihydro-1,4- |
| | benzoxazepin-5-one |
| 148 | 2-[3-(3,4-dihydro-1H-isoquinolin-2-yl)-2-hydroxy-propyl]-4,5-dihydro-3H- |
| | 2-benzazepin-1-one |
| 149 | 4-[(2R)-3-(3,4-dihydro-1H-isoquinolin-2-yl)-2-hydroxy-propyl]-2,3-dihydro- |
| | 1,4-benzoxazepin-5-one |
| 150 | 4-[(2R)-3-(3,4-dihydro-1H-isoquinolin-2-yl)-2-hydroxy-propyl]-8-fluoro- |
| | 2,3-dihydro-1,4-benzoxazepin-5-one |
| 151 | 8-Bromo-4-[(2R)-3-(3,4-dihydro-1H-isoquinolin-2-yl)-2-hydroxy-propyl]- |
| | 2,3-dihydro-1,4-benzoxazepin-5-one |
| 152 | 4-[(2R)-3-(3,4-dihydro-1H-isoquinolin-2-yl)-2-hydroxy-propyl]-2,3- |
| | dihydropyrido[4,3-f][1,4]oxazepin-5-one |
| 153 | 8-Chloro-4-[(2R)-3-(3,4-dihydro-1H-isoquinolin-2-yl)-2-hydroxy-propyl]- |
| | 2,3-dihydropyrido[3,2-f][1,4]oxazepin-5-one |
| 154 | 7-Chloro-2-[(2R)-3-(3,4-dihydro-1H-isoquinolin-2-yl)-2-hydroxy-propyl]- |
| | 4,5-dihydro-3H-2-benzazepin-1-one |
| 155 | 7-Bromo-2-[(2R)-3-(3,4-dihydro-1H-isoquinolin-2-yl)-2-hydroxy-propyl]- |
| | 4,5-dihydro-3H-2-benzazepin-1-one |
| 156 | 8-Bromo-4-[(2R)-3-(3,4-dihydro-1H-isoquinolin-2-yl)-2-hydroxy-propyl]-1- |
| | methyl-2,3-dihydro-1,4-benzodiazepin-5-one |
| 157 | 8-Bromo-4-[(2R)-3-(3,4-dihydro-1H-isoquinolin-2-yl)-2-hydroxy-propyl]-1- |
| | ethyl-2,3-dihydro-1,4-benzodiazepin-5-one |
| 158 | 8-Bromo-4-[(2R)-3-(3,4-dihydro-1H-isoquinolin-2-yl)-2-hydroxy-propyl]-1- |
| | ethyl-3H-1,4-benzodiazepine-2,5-dione |
| 159 | 2-[4-[[4-(2R)-3-(3,4,-dihydro-1H-isoquinolin-2-yl)-2-hydroxy-propyl]-5- |
| | oxo-2,3,-dihydro-1,4,-benzoxazepin-8-yl]-1-piperidyl]acetonitrile |
| 160 | 8-[[1-(2,2-difluoroacetyl)-4-piperidyl]oxy]-4-[(2R)-3-(3,4-dihydro-1H- |
| | isoquinolin-2-yl)-2-hydroxy-propyl]-2,3-dihydro-1,4-benzoxazepin-5-one |
| 161 | 8-[1-(2,2-difluoroacetyl)azetidin-3-yl]oxy-4-[(2R)-3-(3,4-dihydro-1H- |
| | isoquinolin-2-yl)-2-hydroxy-propyl]-2,3-dihydro-1,4-benzoxazepin-5-one |
| 162 | 4-[(2R)-3-(3,4-dihydro-1H-isoquinolin-2-yl)-2-hydroxy-propyl]-8-[[1-(2- |
| | hydroxyethyl)-4-piperidyl]oxy]-2,3-dihydro-1,4-benzoxazepin-5-one |
| 163 | 4-[(2R)-3-(3,4-dihydro-1H-isoquinolin-2-yl)-2-hydroxy-propyl]-8-[[1-(2- |
| | fluoroethyl)-4-piperidyl]oxy]-2,3-dihydro-1,4-benzoxazepin-5-one |
| 164 | 4-[(2R)-3-(3,4-dihydro-1H-isoquinolin-2-yl)-2-hydroxy-propyl]-8-[[1-(2- |
| | fluoroethyl)-4-piperidyl]oxy]-2,3-dihydropyrido[3,2-f][1,4]oxazepin-5-one |
| 165 | 4-[(2R)-3-(3,4-dihydro-1H-isoquinolin-2-yl)-2-hydroxypropyl]-8-[[1-[(3- |
| | methyloxetan-3-yl)methyl]-4-piperidyl]oxy]-2,3-dihydro-1,4-benzoxazepin- |
| | 5-one |
| 166 | 4-[[4-[(2R)-3-(3,4-dihydro-1H-isoquinolin-2-yl)-2-hydroxy-propyl]-5-oxo- |
| | 2,3-dihydro-1,4-benzoazepin-8-yl]oxy]piperidine-1-carbonitrile |
| 167 | 8-[(4-acetylpiperazin-1-yl)methyl]-4-[(2R)-3-(3,4-dihydro-1H-isoquinolin-2- |
| | yl)-2-hydroxy-propyl]-2,2-dimethyl-3H-1,4-benzoxazepin-5-one |
| 168 | 3-[[4-[(2R)-3-(3,4-dihydro-1H-isoquinolin-2-yl)-2-hydroxy-propyl]-2,2- |
| | dimethyl-5-oxo-3H-1,4-benzoxazepin-8-yl]oxy]azetidine-1-carbaldehyde |
| 169 | 4-[[4-[(2R)-3-(3,4-dihydro-1H-isoquinolin-2-yl)-2-hydroxy-propyl]-2,2- |
| | dimethyl-5-oxo-3H-1,4-benzoxazepin-8-yl]methyl]piperazine-1- |
| | carbaldehyde |
| 170 | 4-[(2R)-3-(3,4-dihydro-1H-isoquinolin-2-yl)-2-hydroxy-propyl]-2,2- |
| | dimethyl-8-(2-oxa-6-azaspiro[3.3]heptan-6-ylmethyl)-3H-1,4-benzoxazepin- |
| | 5-one |
| 171 | 4-[(2R)-3-(3,4-dihydro-1H-isoquinolin-2-yl)-2-hydroxy-propyl]-8-[(8-ethyl- |
| | 8-azabicyclo[3.2.1]octan-3-yl)oxy]-2,3-dihydro-1,4-benzoxazepin-5-one |
| 172 | 4-[(2R)-3-(3,4-dihydro-1H-isoquinolin-2-yl)-2-hydroxy-propyl]-8-[(2-ethyl- |
| | 2-azaspiro[3.3]heptan-6-yl)oxy]-2,3-dihydro-1,4-benzoxazepin-5-one |
| 173 | 8-[(8-acetyl-8-azabicyclo[3.2.1]octan-3-yl)oxy]-4-[(2R)-3-(3,4-dihydro-1H- |
| | isoquinolin-2-yl)-2-hydroxy-propyl]-2,3-dihydro-1,4-benzoxazepin-5-one |
| 174 | 8-[(2-acetyl-2-azaspiro[3.3]heptan-6-yl)oxy]-4-[(2R)-3-(3,4-dihydro-1H- |
| | isoquinolin-2-yl)-2-hydroxy-propyl]-2,3-dihydro-1,4-benzoxazepin-5-one |
| 175 | 4-[(2R)-3-(3,4-dihydro-1H-isoquinolin-2-yl)-2-hydroxy-propyl]-8- |
| | (pyrrolidine-1-carbonyl)-2,3-dihydro-1,4-benzoxazepin-5-one |
| 176 | 4-[(2R)-3-(3,4-dihydro-1H-isoquinolin-2-yl)-2-hydroxy-propyl]-8- |
| | (piperidine-1-carbonyl)-2,3-dihydro-1,4-benzoxazepin-5-one |
| 177 | 8-(3,3-Difluoropyrrolidine-1-carbonyl)-4-[(2R)-3-(3,4-dihydro-1H- |
| | isoquinolin-2-yl)-2-hydroxy-propyl]-2,3-dihydro-1,4-benzoxazepin-5-one |
| 178 | 4-[(2R)-3-(3,4-dihydro-1H-isoquinolin-2-yl)-2-hydroxy-propyl]-8-(4- |
| | methylpiperazine-1-carbonyl)-2,3-dihydro-1,4-benzoxazepin-5-one |
| 179 | 4-[(2R)-3-(3,4-dihydro-1H-isoquinolin-2-yl)-2-hydroxy-propyl]-2,2- |
| | dimethyl-8-(pyrrolidin-1-ylmethyl)-3H-1,4-benzoxazepin-5-one |
| 180 | 8-[(1-acetyl-4-piperidyl)oxy]-4-[(2R)-3-(3,4-dihydro-1H-isoquinolin-2-yl)-2- |
| | hydroxy-propyl]-2,2-dimethyl-3H-1,4-benzoxazepin-5-one |
| 181 | 4-[[4-[(2R)-3-(3,4-dihydro-1H-isoquinolin-2-yl)-2-hydroxy-propyl]-2,2- |
| | dimethyl-5-oxo-3H-1,4-benzoxazepin-8-yl]oxy]piperidine-1-carbaldehyde |
| 182 | 4-[(2R)-3-(3,4-dihydro-1H-isoquinolin-2-yl)-2-hydroxy-propyl]-2,2- |
| | dimethyl-8-[[4-(oxetan-3-yl)piperazin-1-yl]methyl]-3H-1,4-benzoxazepin-5- |
| | one |
| 183 | 4-[(2R)-3-(3,4-dihydro-1H-isoquinolin-2-yl)-2-hydroxy-propyl]-2,2- |
| | dimethyl-8-(2-oxa-7-azaspiro[3.4]octan-7-ylmethyl)-3H-1,4-benzoxazepin- |
| | 5-one |
| 184 | 4-[(2R)-3-(3,4-dihydro-1H-isoquinolin-2-yl)-2-hydroxy-propyl]-8-(4- |
| | fluoropiperidine-1-carbonyl)-2,3-dihydro-1,4-benzoxazepin-5-one |
| 185 | 4-[(2R)-3-(3,4-dihydro-1H-isoquinolin-2-yl)-2-hydroxy-propyl]-8-[3- |
| | (trifluoromethyl)piperidine-1-carbonyl]-2,3-dihydro-1,4-benzoxazepin-5-one |
| 186 | 4-[(2R)-3-(3,4-dihydro-1H-isoquinolin-2-yl)-2-hydroxy-propyl]-8-[4- |
| | (trifluoromethyl)piperidine-1-carbonyl]-2,3-dihydro-1,4-benzoxazepin-5-one |
| 187 | 4-[(2R)-3-(3,4-dihydro-1H-isoquinolin-2-yl)-2-hydroxy-propyl]-8-(2,6- |
| | dimethylmorpholine-4-carbonyl)-2,3-dihydro-1,4-benzoxazepin-5-one |
| 188 | 8-(2-Azabicyclo[2.2.1]heptane-2-carbonyl)-4-[(2R)-3-(3,4-dihydro-1H- |
| | isoquinolin-2-yl)-2-hydroxy-propyl]-2,3-dihydro-1,4-benzoxazepin-5-one |
| 189 | 4-[(2R)-3-(3,4-dihydro-1H-isoquinolin-2-yl)-2-hydroxy-propyl]-8-(3-oxa-8- |
| | azabicyclo[3.2.1]octane-8-carbonyl)-2,3-dihydro-1,4-benzoxazepin-5-one |
| 190 | 4-[(2R)-3-(3,4-dihydro-1H-isoquinolin-2-yl)-2-hydroxy-propyl]-8-(2- |
| | methylmorpholine-4-carbonyl)-2,3-dihydro-1,4-benzoxazepin-5-one |
| 191 | 4-[(2R)-3-(3,4-dihydro-1H-isoquinolin-2-yl)-2-hydroxy-propyl]-8-(3- |
| | methylmorpholine-4-carbonyl)-2,3-dihydro-1,4-benzoxazepin-5-one |
| 192 | 4-[(2R)-3-(3,4-dihydro-1H-isoquinolin-2-yl)-2-hydroxy-propyl]-2,2- |
| | dimethyl-8-(morpholinomethyl)-3H-pyrido[3,2-f][1,4]oxazepin-5-one |
| 193 | 8-(3,3a,4,5,6,6a-Hexahydro-1H-cyclopenta[c]pyrrole-2-carbonyl)-4-[(2R)-3- |
| | (3,4-dihydro-1H-isoquinolin-2-yl)-2-hydroxy-propyl]-2,3-dihydro-1,4- |
| | benzoxazepin-5-one |
| 194 | 4-[(2R)-3-(3,4-dihydro-1H-isoquinolin-2-yl)-2-hydroxy-propyl]-8-(3- |
| | fluoropiperidine-1-carbonyl)-2,3-dihydro-1,4-benzoxazepin-5-one |
| 195 | 8-(2,2-Difluoromorpholine-4-carbonyl)-4-[(2R)-3-(3,4-dihydro-1H- |
| | isoquinolin-2-yl)-2-hydroxy-propyl]-2,3-dihydro-1,4-benzoxazepin-5-one |
| 196 | 8-(4,4-difluoropiperidine-1-carbonyl)-4-[(2R)-3-(3,4-dihydro-1H- |
| | isoquinolin-2-yl)-2-hydroxy-propyl]-2,3-dihydro-1,4-benzoxazepin-5-one |
| 197 | 4-[(2R)-3-(3,4-dihydro-1H-isoquinolin-2-yl)-2-hydroxy-propyl]-2,2- |
| | dimethyl-8-(1-piperidylmethyl)-3H-pyrido[3,2-f][1,4]oxazepin-5-one |
| 198 | 4-[(2R)-3-(3,4-dihydro-1H-isoquinolin-2-yl)-2-hydroxypropyl]-8-[4- |
| | [methyl(oxetan-3-yl)amino]cyclohexoxy]-2,3-dihydro-1,4-benzoxazepin-5- |
| | one |
| 199 | 4-[(2R)-3-(3,4-dihydro-1H-isoquinolin-2-yl)-2-hydroxypropyl]-8-[4-[2- |
| | fluoroethyl(methyl)amino]cyclohexoxy]-2,3-dihydro-1,4-benzoxazepin-5- |
| | one |
| 200 | (2S)-4-[(2R)-3-(3,4-dihydro-1H-isoquinolin-2-yl)-2-hydroxy-propyl]-8-[(1- |
| | ethanethioyl-4-piperidyl)oxy]-2-methyl-2,3-dihydro-1,4-benzoxazepin-5-one |
| 201 | (2S)-4-[(2R)-3-(3,4-dihydro-1H-isoquinolin-2-yl)-2-hydroxy-propyl]-8-[1- |
| | (2-fluoroethyl)azetidin-3-yl]oxy-2-methyl-2,3-dihydro-1,4-benzoxazepin-5- |
| | one |
| 202 | (2S)-4-[(2R)-3-(3,4-dihydro-1H-isoquinolin-2-yl)-2-hydroxy-propyl]-8-[1- |
| | (2-fluoroethyl)-4-piperidyl]oxy-2-methyl-2,3-dihydro-1,4-benzoxazepin-5- |
| | one |
| 203 | 4-[(2R)-3-(3,4-dihydro-1H-isoquinolin-2-yl)-2-hydroxy-propyl]-2,2- |
| | dimethyl-8-(3-oxa-8-azabicyclo[3.2.1]octane-8-carbonyl)-3H-1,4- |
| | benzoxazepin-5-one |
| 204 | 4-[(2R)-3-(3,4-dihydro-1H-isoquinolin-2-yl)-2-hydroxy-propyl]-2,2- |
| | dimethyl-8-(2-oxa-5-azabicyclo[2.2.1]heptane-5-carbonyl)-3H-1,4- |
| | benzoxazepin-5-one |
| 205 | 4-[(2R)-3-(3,4-dihydro-1H-isoquinolin-2-yl)-2-hydroxy-propyl]-8-(3,5- |
| | dimethylmorpholine-4-carbonyl)-2,2-dimethyl-3H-1,4-benzoxazepin-5-one |
| 206 | 4-[(2R)-3-(3,4-dihydro-1H-isoquinolin-2-yl)-2-hydroxy-propyl]-8-[(3- |
| | fluoro-1-methyl-4-piperidyl)oxy]-2,3-dihydro-1,4-benzoxazepin-5-one |
| 207 | 4-[(2R)-3-(3,4-dihydro-1H-isoquinolin-2-yl)-2-hydroxy-propyl]-8-[[3- |
| | fluoro-1-(2-fluoroethyl)-4-piperidyl]oxy]-2,3-dihydro-1,4-benzoxazepin-5- |
| | one |
| 208 | 4-[(2R)-3-(3,4-dihydro-1H-isoquinolin-2-yl)-2-hydroxy-propyl]-8-[[3- |
| | fluoro-1-(2-hydroxyethyl)-4-piperidyl]oxy]-2,3-dihydro-1,4-benzoxazepin- |
| | 5-one |
| 209 | 4-[(2R)-3-(3,4-dihydro-1H-isoquinolin-2-yl)-2-hydroxy-propyl]-8-(3,4- |
| | dihydro-1H-pyrrolo[1,2-a]pyrazine-2-carbonyl)-2,2-dimethyl-3H-1,4- |
| | benzoxazepin-5-one |
| 210 | 8-(3,4,6,7,8,8a-Hexahydro-1H-pyrrolo[1,2-a]pyrazine-2-carbonyl)-4-[(2R)- |
| | 3-(3,4-dihydro-1H-isoquinolin-2-yl)-2-hydroxy-propyl]-2,2-dimethyl-3H- |
| | 1,4-benzoxazepin-5-one |
| 211 | 4-[(2R)-3-(3,4-dihydro-1H-isoquinolin-2-yl)-2-hydroxy-propyl]-8-(5- |
| | fluoropyrimidin-2-yl)oxy-2,3-dihydro-1,4-benzoxazepin-5-one |
| 212 | 4-[(2R)-3-(3,4-dihydro-1H-isoquinolin-2-yl)-2-hydroxy-propyl]-8-[[1-(2- |
| | hydroxypropyl)-4-piperidyl]oxy]-2,3-dihydro-1,4-benzoxazepin-5-one |
| 213 | 4-[(2R)-3-(3,4-dihydro-1H-isoquinolin-2-yl)-2-hydroxy-propyl]-8-[(1- |
| | methyl-3-piperidyl)oxy]-2,3-dihydro-1,4-benzoxazepin-5-one |
| 214 | 4-[(2R)-3-(3,4-dihydro-1H-isoquinolin-2-yl)-2-hydroxy-propyl]-8- |
| | propanoyl-2,3-dihydro-1,4-benzoxazepin-5-one |
| 215 | 4-[(2R)-3-(3,4-dihydro-1H-isoquinolin-2-yl)-2-hydroxy-propyl]-8-[[8-(2- |
| | fluoroethyl)-8-azabicyclo[3.2.1]octan-3-yl]oxy]-2,3-dihydro-1,4- |
| | benzoxazepin-5-one |
| 216 | 8-(Cyclopropanecarbonyl)-4-[(2R)-3-(3,4-dihydro-1H-isoquinolin-2-yl)-2- |
| | hydroxy-propyl]-2,3-dihydro-1,4-benzoxazepin-5-one |
| 217 | 8-(Cyclopentanecarbonyl)-4-[(2R)-3-(3,4-dihydro-1H-isoquinolin-2-yl)-2- |
| | hydroxy-propyl]-2,3-dihydro-1,4-benzoxazepin-5-one |
| 218 | 4-[(2R)-3-(3,4-dihydro-1H-isoquinolin-2-yl)-2-hydroxypropyl]-8-[(1- |
| | methyl-3-piperidyl)oxy]-2,3-dihydro-1,4-benzoxazepin-5-one |
| 219 | (2R)-4-[(2R)-3-(3,4-dihydro-1H-isoquinolin-2-yl)-2-hydroxy-propyl]-8-[[1- |
| | (2-hydroxyethyl)-4-piperidyl]oxy]-2-methyl-2,3-dihydro-1,4-benzoxazepin- |
| | 5-one |
| 220 | (2R)-4-[(2R)-3-(3,4-dihydro-1H-isoquinolin-2-yl)-2-hydroxy-propyl]-8-[[1- |
| | (2-hydroxy-2-methyl-propyl)-4-piperidyl]oxy]-2-methyl-2,3-dihydro-1,4- |
| | benzoxazepin-5-one |
| 221 | 4-[(2R)-3-(3,4-dihydro-1H-isoquinolin-2-yl)-2-hydroxy-propyl]-8-(1- |
| | morpholinoethyl)-2,3-dihydro-1,4-benzoxazepin-5-one |
| 222 | 4-[(2R)-3-(3,4-dihydro-1H-isoquinolin-2-yl)-2-hydroxy-propyl]-8-[1-(2-oxa- |
| | 6-azaspiro[3.3]heptan-6-yl)ethyl]-2,3-dihydro-1,4-benzoxazepin-5-one |
| 223 | 4-[(2R)-3-(3,4-dihydro-1H-isoquinolin-2-yl)-2-hydroxy-propyl]-8-(3- |
| | hydroxyprop-1-ynyl)-2,3-dihydro-1,4-benzoxazepin-5-one |
| 224 | (2R)-4-[(2R)-3-(3,4-dihydro-1H-isoquinolin-2-yl)-2-hydroxy-propyl]-8-[[1- |
| | (2-methoxyethyl)-4-piperidyl]oxy]-2-methyl-2,3-dihydro-1,4-benzoxazepin- |
| | 5-one |
| 225 | 4-[(2R)-3-(3,4-dihydro-1H-isoquinolin-2-yl)-2-hydroxy-propyl]-8-(4- |
| | pyridyl)-2,3-dihydro-1,4-benzoxazepin-5-one |
| 226 | 4-[(2R)-3-(3,4-dihydro-1H-isoquinolin-2-yl)-2-hydroxy-propyl]-8-[2-(4- |
| | pyridyl)ethynyl]-2,3-dihydro-1,4-benzoxazepin-5-one |
| 227 | 4-[(2R)-3-(3,4-dihydro-1H-isoquinolin-2-yl)-2-hydroxy-propyl]-8-[2-(3- |
| | pyridyl)ethynyl]-2,3-dihydro-1,4-benzoxazepin-5-one |
| 228 | 4-[(2R)-3-(3,4-dihydro-1H-isoquinolin-2-yl)-2-hydroxy-propyl]-8-(3- |
| | hydroxybut-1-ynyl)-2,3-dihydro-1,4-benzoxazepin-5-one |
| 229 | 4-[(2R)-3-(3,4-dihydro-1H-isoquinolin-2-yl)-2-hydroxy-propyl]-8-[3- |
| | (methylamino)prop-1-ynyl]-2,3-dihydro-1,4-benzoxazepin-5-one |
| 230 | 4-[(2R)-3-(3,4-dihydro-1H-isoquinolin-2-yl)-2-hydroxy-propyl]-8-(3- |
| | pyridyl)-2,3-dihydro-1,4-benzoxazepin-5-one |
| 231 | 4-[(2R)-3-(3,4-dihydro-1H-isoquinolin-2-yl)-2-hydroxy-propyl]-8-(1,3- |
| | dimethylpyrazol-4-yl)-2,3-dihydro-1,4-benzoxazepin-5-one |
| 232 | 4-[(2R)-3-(3,4-dihydro-1H-isoquinolin-2-yl)-2-hydroxy-propyl]-8-(1- |
| | methylpyrazol-4-yl)-2,3-dihydro-1,4-benzoxazepin-5-one |
| 233 | 4-[(2R)-3-(3,4-dihydro-1H-isoquinolin-2-yl)-2-hydroxy-propyl]-8-(1,5- |
| | dimethylpyrazol-4-yl)-2,3-dihydro-1,4-benzoxazepin-5-one |
| 234 | 4-[(2R)-3-(3,4-dihydro-1H-isoquinolin-2-yl)-2-hydroxy-propyl]-8-[[1-(2- |
| | hydroxyethyl)-4-piperidyl]oxy]-2,2-dimethyl-3H-pyrido[3,2-f][1,4]oxazepin- |
| | 5-one |
| 235 | 4-[(2R)-3-(3,4-dihydro-1H-isoquinolin-2-yl)-2-hydroxy-propyl]-2,2- |
| | dimethyl-8-[[(3R)-1-methyl-3-piperidyl]oxy]-3H-pyrido[3,2- |
| | f][1,4]oxazepin-5-one |
| 236 | 4-[(2R)-3-(3,4-dihydro-1H-isoquinolin-2-yl)-2-hydroxy-propyl]-8-[[(1-(2- |
| | fluoroethyl)-3-piperidyl]oxy]-2,3-dihydro-1,4-benzoxazepin-5-one |
| 237 | 4-[(2R)-3-(3,4-dihydro-1H-isoquinolin-2-yl)-2-hydroxy-propyl]-8-[[(1-(2- |
| | hydroxyethyl)-3-piperidyl]oxy]-2,3-dihydro-1,4-benzoxazepin-5-one |
| 238 | 4-[(2R)-3-(3,4-dihydro-1H-isoquinolin-2-yl)-2-hydroxy-propyl]-8-[[1-(2- |
| | hydroxy-2-methyl-propyl)-4-piperidyl]oxy]-2,3-dihydro-1,4-benzoxazepin- |
| | 5-one |
| 239 | 4-[(2R)-3-(3,4-dihydro-1H-isoquinolin-2-yl)-2-hydroxy-propyl]-8-(6-fluoro- |
| | 2-pyridyl)-2,3-dihydro-1,4-benzoxazepin-5-one |
| 240 | (2R)-4-[(2R)-3-(3,4-dihydro-1H-isoquinolin-2-yl)-2-hydroxy-propyl]-8-[[1- |
| | (2-ethoxyethyl)-4-piperidyl]oxy]-2-methyl-2,3-dihydro-1,4-benzoxazepin-5- |
| | one |
| 241 | (2R)-4-[(2R)-3-(3,4-dihydro-1H-isoquinolin-2-yl)-2-hydroxy-propyl]-8-[[8- |
| | (2-hydroxyethyl)-8-azabicyclo[3.2.1]octan-3-yl]oxy]-2-methyl-2,3-dihydro- |
| | 1,4-benzoxazepin-5-one |
| 242 | (2R)-4-[(2R)-3-(3,4-dihydro-1H-isoquinolin-2-yl)-2-hydroxy-propyl]-8-[[8- |
| | (2-fluoroethyl)-8-azabicyclo[3.2.1]octan-3-yl]oxy]-2-methyl-2,3-dihydro- |
| | 1,4-benzoxazepin-5-one |
| 243 | 4-[(2R)-3-(3,4-dihydro-1H-isoquinolin-2-yl)-2-hydroxy-propyl]-8-[[1-(2- |
| | methoxyethyl)-4-piperidyl]oxy]-2,3-dihydro-1,4-benzoxazepin-5-one |
| 244 | 4-[(2R)-3-(3,4-dihydro-1H-isoquinolin-2-yl)-2-hydroxy-propyl]-8-(6- |
| | methoxy-2-pyridyl)-2,3-dihydro-1,4-benzoxazepin-5-one |
| 245 | (2R)-4-[(2R)-3-(3,4-dihydro-1H-isoquinolin-2-yl)-2-hydroxy-propyl]-8-[[1- |
| | (2-hydroxypropyl)-4-piperidyl]oxy]-2-methyl-2,3-dihydro-1,4- |
| | benzoxazepin-5-one |
| 246 | 4-[(2R)-3-(3,4-dihydro-1H-isoquinolin-2-yl)-2-hydroxy-propyl]-2,2- |
| | dimethyl-8-(1-morpholinoethyl)-3H-pyrido[3,2-f][1,4]oxazepin-5-one |
| 247 | 4-[(2R)-3-(3,4-dihydro-1H-isoquinolin-2-yl)-2-hydroxy-propyl]-2,2- |
| | dimethyl-8-[1-(2-oxa-6-azaspiro[3.3]heptan-6-yl)ethyl]-3H-pyrido[3,2- |
| | f][1,4]oxazepin-5-one |
| 248 | 4-[(2R)-3-(3,4-dihydro-1H-isoquinolin-2-yl)-2-hydroxy-propyl]-8-[[1-(2- |
| | hydroxypropyl)-4-piperidyl]oxy]-2,2-dimethyl-3H-pyrido[3,2- |
| | f][1,4]oxazepin-5-one |
| 249 | 4-[(2R)-3-(3,4-dihydro-1H-isoquinolin-2-yl)-2-hydroxypropyl]-8-(3,4- |
| | dihydro-1H-pyrrolo[1,2-a]pyrazin-2-ylmethyl)-2,3-dihydro-1,4- |
| | benzoxazepin-5-one |
| 250 | (2R)-4-[(2R)-3-(3,4-dihydro-1H-isoquinolin-2-yl)-2-hydroxypropyl]-2- |
| | methyl-8-(2-oxa-6-azaspiro[3.3]heptan-6-ylmethyl)-2,3-dihydro-1,4- |
| | benzoxazepin-5-one |
| 251 | 4-[(2R)-3-(3,4-dihydro-1H-isoquinolin-2-yl)-2-hydroxypropyl]-8-(2-oxa-7- |
| | azaspiro[3.5]nonan-7-ylmethyl)-2,3-dihydro-1,4-benzoxazepin-5-one |
| 252 | (2R)-4-[(2R)-3-(3,4-dihydro-1H-isoquinolin-2-yl)-2-hydroxypropyl]-2- |
| | methyl-8-(2-oxa-7-azaspiro[3.5]nonan-7-ylmethyl)-2,3-dihydro-1,4- |
| | benzoxazepin-5-one |
| 253 | (2R)-4-[(2R)-3-(3,4-dihydro-1H-isoquinolin-2-yl)-2-hydroxy-propyl]-8- |
| | [[(3R,4R)-3-fluoro-1-(2-hydroxyethyl)-4-piperidyl]oxy]-2-methyl-2,3- |
| | dihydro-1,4-benzoxazepin-5-one 2 hydrochloride |
| 254 | (2R)-4-[(2R)-3-(3,4-dihydro-1H-isoquinolin-2-yl)-2-hydroxy-propyl]-8- |
| | [[(3R,4R)-3-fluoro-1-(2-hydroxypropyl)-4-piperidyl]oxy]-2-methyl-2,3- |
| | dihydro-1,4-benzoxazepin-5-one 2 hydrochloride |
| 255 | (2R)-4-[(2R)-3-(3,4-dihydro-1H-isoquinolin-2-yl)-2-hydroxy-propyl]-8- |
| | [[(3R,4R)-3-fluoro-1-(2-hydroxyethyl)-4-piperidyl]oxy]-2-methyl-2,3- |
| | dihydro-1,4-benzoxazepin-5-one 2 hydrochloride |
| 256 | 8-[[3,3-difluoro-1-(2-hydroxyethyl)-4-piperidyl]oxy]-4-[(2R)-3-(3,4- |
| | dihydro-1H-isoquinolin-2-yl)-2-hydroxy-propyl]-2,2-dimethyl-3H- |
| | pyrido[3,2-f][1,4]oxazepin-5-one |
| 257 | 4-[(2R)-3-(3,4-dihydro-1H-isoquinolin-2-yl)-2-hydroxypropyl]-8-[1-(1- |
| | piperidyl)ethyl]-2,3-dihydro-1,4-benzoxazepin-5-one |
| 258 | 4-[(2R)-3-(3,4-dihydro-1H-isoquinolin-2-yl)-2-hydroxypropyl]-8-[1-(4- |
| | hydroxy-1-piperidyl)ethyl]-2,3-dihydro-1,4-benzoxazepin-5-one |
| 259 | 4-[(2R)-3-(3,4-dihydro-1H-isoquinolin-2-yl)-2-hydroxypropyl]-8-[1-(4- |
| | methoxy-1-piperidyl)ethyl]-2,3-dihydro-1,4-benzoxazepin-5-one |
| 260 | 4-[(2R)-3-(3,4-dihydro-1H-isoquinolin-2-yl)-2-hydroxypropyl]-8-[1-(3- |
| | hydroxy-3-methyl-pyrrolidin-1-yl)ethyl]-2,3-dihydro-1,4-benzoxazepin-5- |
| | one |
| 261 | (2R)-4-[(2R)-3-(3,4-dihydro-1H-isoquinolin-2-yl)-2-hydroxy-propyl]-8-[[1- |
| | (2-hydroxyethyl)-4-piperidyl]oxy]-2-methyl-2,3-dihydro-1,4-benzoxazepin- |
| | 5-one 2 hydrochloride |
| 262 | 4-[(2R)-3-(3,4-dihydro-1H-isoquinolin-2-yl)-2-hydroxy-propyl]-8-[[1-(2- |
| | methoxyethyl)-4-piperidyl]oxy]-2,2-dimethyl-3H-pyrido[3,2- |
| | f][1,4]oxazepin-5-one |
| 263 | 4-[(2R)-3-(3,4-dihydro-1H-isoquinolin-2-yl)-2-hydroxy-propyl]-8-[(3S)-1- |
| | (oxetan-3-yl)pyrrolidin-3-yl]oxy-2,3-dihydro-1,4-benzoxazepin-5-one |
| 264 | (2R)-4-[(2R)-3-(3,4-dihydro-1H-isoquinolin-2-yl)-2-hydroxy-propyl]-2- |
| | methyl-8-[(3S)-1-methylpyrrolidin-3-yl]oxy-2,3-dihydro-1,4-benzoxazepin- |
| | 5-one |
| 265 | 4-[(2R)-3-(3,4-dihydro-1H-isoquinolin-2-yl)-2-hydroxy-propyl]-8-[(3R)- |
| | tetrahydrofuran-3-yl]oxy-2,3-dihydro-1,4-benzoxazepin-5-one |
| 266 | 4-[(2R)-3-(3,4-dihydro-1H-isoquinolin-2-yl)-2-hydroxy-propyl]-8-[(3S)- |
| | tetrahydrofuran-3-yl]oxy-2,3-dihydro-1,4-benzoxazepin-5-one |
| 267 | (2R)-4-[(2R)-3-(3,4-dihydro-1H-isoquinolin-2-yl)-2-hydroxy-propyl]-8-[[1- |
| | [(2S)-2-hydroxypropyl]-4-piperidyl]oxy]-2-methyl-2,3-dihydro-1,4- |
| | benzoxazepin-5-one |
| 268 | (2R)-4-[(2R)-3-(3,4-dihydro-1H-isoquinolin-2-yl)-2-hydroxy-propyl]-8-[[1- |
| | [(2R)-2-hydroxypropyl]-4-piperidyl]oxy]-2-methyl-2,3-dihydro-1,4- |
| | benzoxazepin-5-one |
| 269 | 8-[Cyclopropyl(hydroxy)methyl]-4-[(2R)-3-(3,4-dihydro-1H-isoquinolin-2- |
| | yl)-2-hydroxy-propyl]-2,3-dihydro-1,4-benzoxazepin-5-one |
| 270 | 8-[Cyclopentyl(hydroxy)methyl]-4-[(2R)-3-(3,4-dihydro-1H-isoquinolin-2- |
| | yl)-2-hydroxy-propyl]-2,3-dihydro-1,4-benzoxazepin-5-one |
| 271 | 4-[(2R)-3-(3,4-dihydro-1H-isoquinolin-2-yl)-2-hydroxy-propyl]-8-[(3R)-1- |
| | (oxetan-3-yl)pyrrolidin-3-yl]oxy-2,3-dihydro-1,4-benzoxazepin-5-one |
| 272 | 4-[(2R)-3-(3,4-dihydro-1H-isoquinolin-2-yl)-2-hydroxy-propyl]-8-[(3R)-1- |
| | (2-methoxyethyl)pyrrolidin-3-yl]oxy-2,3-dihydro-1,4-benzoxazepin-5-one |
| 273 | (2R)-4-[(2R)-3-(3,4-dihydro-1H-isoquinolin-2-yl)-2-hydroxy-propyl]-2- |
| | methyl-8-[(3R)-1-methylpyrrolidin-3-yl]oxy-2,3-dihydro-1,4-benzoxazepin- |
| | 5-one |
| 274 | (2R)-4-[(2R)-3-(3,4-dihydro-1H-isoquinolin-2-yl)-2-hydroxy-propyl]-8- |
| | [(3R)-1-(2-methoxyethyl)pyrrolidin-3-yl]oxy-2-methyl-2,3-dihydro-1,4- |
| | benzoxazepin-5-one |
| 275 | 4-[(2R)-3-(3,4-dihydro-1H-isoquinolin-2-yl)-2-hydroxy-propyl]-8-[[1-(3,3,3- |
| | trifluoro-2-hydroxy-propyl)-4-piperidyl]oxy]-2,3-dihydro-1,4-benzoxazepin- |
| | 5-one |
| 276 | (2R)-4-[(2R)-3-(3,4-dihydro-1H-isoquinolin-2-yl)-2-hydroxy-propyl]-2- |
| | methyl-8-[[1-(3,3,3-trifluoro-2-hydroxy-propyl)-4-piperidyl]oxy]-2,3- |
| | dihydro-1,4-benzoxazepin-5-one |
| 277 | (2R)-4-[(2R)-3-(3,4-dihydro-1H-isoquinolin-2-yl)-2-hydroxypropyl]-2- |
| | methyl-8-[(3R)-1-(oxetan-3-yl)pyrrolidin-3-yl]oxy-2,3-dihydro-1,4- |
| | benzoxazepin-5-one |

According to another embodiment disclosed herein, the PRMT5 inhibitor may be 4-[(2R)-3-(3,4-dihydro-1H-isoquinolin-2-yl)-2-hydroxy-propyl]-8-[(1-methyl-4-piperidyl)oxy]-2,3-dihydro-1,4-benzoxazepin-5-one [Compound-87] or a pharmaceutically acceptable salt thereof.

In another embodiment disclosed herein, the PRMT5 inhibitor may be 4-[(2R)-3-(3,4-dihydro-1H-isoquinolin-2-yl)-2-hydroxy-propyl]-2,2-dimethyl-8-[(1-methyl-4-piperidyl)oxy]-3H-1,4-benzoxazepin-5-one [Compound-112] or a pharmaceutically acceptable salt thereof.

In another embodiment disclosed herein, the PRMT5 inhibitor may be 4-[(2R)-3-(3,4-dihydro-1H-isoquinolin-2-yl)-2-hydroxy-propyl]-8-[(3R)-1-(oxetan-3-yl)pyrrolidin-3-yl]oxy-2,3-dihydro-1,4-benzoxazepin-5-one [Compound-271] or a pharmaceutically acceptable salt thereof.

According to another embodiment in described herein, there is provided a method for treating and/or preventing cancer in a human subject in need of such treatment and/or prevention, in which the method comprises administering to the human subject a therapeutically effective amount of any of the pharmaceutical compositions disclosed herein.

According to another embodiment disclosed herein, the human subject is a subject in need of treatment and/or prevention of cancer, including a subject having multidrug resistance (MDR), and exhibiting resistance or poor responsiveness to DNA damage inducing agents.

According to another embodiment disclosed herein, the human subject is a subject in need of treatment and/or prevention of cancer, including a subject that is HRD-positive or HRD-negative.

According to another embodiment disclosed herein, the cancer includes, but is not limited to, one or more selected from the group consisting of acoustic neuroma, adenocarcinoma, adrenal cancer, anal cancer, angiosarcoma, benign monoclonal gammopathy, cholangiocarcinoma, bladder cancer, breast cancer, brain cancer, lymphoma, multiple myeloma, lacrimal gland tumor, bronchial cancer, cervical cancer, craniopharyngioma, colorectal cancer, epithelial carcinoma, ependymoma, adenoid cystic carcinoma (ACC), endothelial sarcoma, endometrial cancer, esophageal cancer, Barrett's adenocarcinoma, Ewing's sarcoma, eye cancer, gallbladder cancer, gastric cancer, colon cancer, gastrointestinal stromal tumor (GIST), head and neck cancer, oral cancer (OSCC), throat cancer, hematologic cancer, hemangioblastoma, inflammatory myofibroblastic tumor, immunocyte-associated amyloidosis, kidney cancer, renal cell carcinoma, liver cancer, lung cancer, melanoma, uveal melanoma, acute myeloid leukemia (AML), myelodysplastic syndrome (MDS), mesothelioma, myeloproliferative disorder (MPD), chronic idiopathic myelofibrosis, chronic myeloid leukemia (CML), chronic neutrophilic leukemia (CNL), neuroblastoma, neurofibroma, neuroendocrine cancer, osteosarcoma, ovarian cancer, papillary adenocarcinoma, pancreatic cancer, penile cancer, prostate cancer, rectal cancer, rhabdomyosarcoma, salivary gland cancer, skin cancer, small intestinal cancer, soft tissue sarcoma, thyroid cancer, urethral cancer, vaginal cancer, and vulvar cancer.

The brain cancer includes, but is not limited to, one or more selected from the group consisting of meningioma, glioma, medulloblastoma, glioblastoma, and brain metastasis. The lung cancer also includes, but is not limited to, one or more selected from the group consisting of non-small cell lung cancer (NSCLC) and small cell lung cancer (SCLC).

The breast cancer or ovarian cancer may be a BRCA1 wild-type cancer, a BRCA1 mutant cancer, a BRCA2 wild-type cancer, or a BRCA2 mutant cancer, or may be any one cancer selected from the group consisting of a BRCA1 wild-type and BRCA2 wild-type cancer, a BRCA1 wild-type and BRCA2 mutant cancer, a BRCA1 mutant and BRCA2 wild-type cancer, and a BRCA1 mutant and BRCA2 mutant cancer. In a specific embodiment, the cancer may be homologous recombination deficiency (HRD)-positive or HRD-negative.

In some embodiments, the pharmaceutical composition may include at least one compound for the treatment and/or prevention of cancer which includes one or more compounds selected from the group consisting of 4-[(2R)-3-(3,4-dihydro-1H-isoquinolin-2-yl)-2-hydroxy-propyl]-8-[(1-methyl-4-piperidyl)oxy]-2,3-dihydro-1,4-benzoxazepin-5-one [Compound-87], 4-[(2R)-3-(3,4-dihydro-1H-isoquinolin-2-yl)-2-hydroxy-propyl]-2,2-dimethyl-8-[(1-methyl-4-piperidyl)oxy]-3H-1,4-benzoxazepin-5-one [Compound-112], and 4-[(2R)-3-(3,4-dihydro-1H-isoquinolin-2-yl)-2-hydroxy-propyl]-8-[(3R)-1-(oxetane-3-yl)pyrrolidin-3-yl]oxy-2,3-dihydro-1,4-benzoxazepin-5-one [Compound-271], and may be in combination with at least one DNA damage inducing substances.

In some embodiments, the pharmaceutical combination may comprise at least one PRMT5 inhibitor of Chemical Formula 1 and an alkylating agent among DNA damage inducing substances. In some embodiments, the alkylating agent may be at least one selected from the group consisting of cisplatin (azane; dichloroplatinum), carboplatin (azanide; cyclobutane-1,1-dicarboxylic acid; platinum(2+)), oxaliplatin ([(1R,2R)-2-azanidylcyclohexyl]azanide; oxalic acid; platinum(2+)), temozolomide (3-methyl-4-oxoimidazo[5,1-d][1,2,3,5]tetrazine-8-carboxamide), and lubinectedin ([(1R,2R,3R,11S,12S,14R,26R)-5,12-dihydroxy-6,6′-dimethoxy-7,21,30-trimethyl-27-oxospiro[17,19,28-trioxa-24-thia-13,30-13,30-diazaheptacycle [12.9.6.13,11.02,13.04,9.015,23.016,20]triaconta-4(9),5,7,15,20,22-hexaen-26,1′-2,3,4,9-tetrahydropyrido[3,4-b]indole]-22-yl]acetate).

In some embodiments, the pharmaceutical combination may comprise at least one PRMT5 inhibitor of Chemical Formula 1 and an anti-metabolite among DNA damage inducing substances. In some embodiments, the anti-metabolite may be one or more selected from the group consisting of fluorouracil (5-fluoro-1H-pyrimidine-2,4-dione), capecitabine (pentyl N-[1-[(2R,3R,4S,5R)-3,4-dihydroxy-5-methyloxolan-2-yl]-5-fluoro-2-oxopyrimidin-4-yl]carbamate), gemcitabine(4-amino-1-[(2R,4R,5R)-3,3-difluoro-4-hydroxy-5-(hydroxymethyl)oxolan-2-yl]pyrimidin-2-one), and methotrexate ((2S)-2-[[4-[(2,4-diaminopyrimidin-6-yl)methyl-methylamino]benzoyl]amino]pentanedioicacid).

In some embodiments, the pharmaceutical combination may comprise at least one PRMT5 inhibitor of formula I and a topoisomerase among the DNA damage inducing substances. In some embodiments, the topoisomerase is etoposide ([(1S,2S,3R,4S,7R,9S,10S,12R,15S)-4-acetyloxy-1,9,12-trihydroxy-15-[(2R,3S)-2-hydroxy-3-[(2-methylpropan-2-yl)oxycarbonylamino]-3-phenylpropanoyl]oxy-10,14,17,17-tetramethyl-11-oxo-6-oxatetracyclo[11.3.1.03,10.04,7]heptadec-13-en-2-yl]benzoate), irinotecan ([(19S)-10,19-diethyl-19-hydroxy-14,18-dioxo-17-oxa-3,13-diazapentacyclo[11.8.02,11.04,9.015,20]henicosa-1(21),2,4(9),5,7,10,15(20)-heptaen-7-yl]4-piperidine-1-ylpiperidine-1-carboxylate), SN-38 ((4S)-4,11-diethyl-4,9-dihydroxy-1,4-dihydro-3H,14H-pyrano[3′,4′:6,7]indoleizono[1,2-b]quinoline-3,14-dione), and doxorubicin It may be at least one selected from the group consisting of ((7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione).

In some embodiments, the pharmaceutical combination may comprise at least one PRMT5 inhibitor of Chemical Formula 1 and a microtubule inhibitor among DNA damage inducing substances. In some embodiments, the microtubule inhibitor may be one or more selected from the group consisting of paclitaxel ([(1S,2S,3R,4S,7R,9S,10S,12R,15S)-4,12-diacetyloxy-15-[(2R,3S)-3-benzamido-2-hydroxy-3-phenylpropanoyl]oxy-1,9-dihydroxy-10,14,17,17-tetramethyl-11-oxo-6-oxatetracyclo[11.3.1.03,10.04,7]heptadeca-13-en-2-yl]benzoate), and docetaxel ([(1S,2S,3R,4S,7R,9S,10S,12R,15S)-4-acetyloxy-1,9,12-trihydroxy-15-[(2R,3S)-2-hydroxy-3-[(tert-butoxycarbonylamino]-3-phenylpropanoyl]oxy-10,14,17,17-tetramethyl-11-oxo-6-oxatetracyclo[11.3.1.03,10,04,7]heptadeca-13-en-2-yl]benzoate).

In some embodiments, the pharmaceutical combination may comprise at least one PRMT5 inhibitor of Chemical Formula 1 and a PARP inhibitor. In some embodiments, the PARP inhibitor may be at least one selected from the group consisting of olaparib, veliparib, niraparib, pamiparib, talazoparib, rucaparib, CEP-9722, E7016 (GPI 21016), fluzoparib, AZD5305, and AZD9574.

In the present disclosure, when the pharmaceutical combination comprises at least one PRMT5 inhibitor of Chemical Formula 1 and a PARP inhibitor among DNA damage inducing substances, the pharmaceutical combination may be used for the treatment of cancer, BRCA mutation-positive cancer or BRCA mutation-negative cancer, and the like, for example, for the treatment of BRCA-mutated ovarian cancer.

In some embodiments, administration of the PRMT5 inhibitor of Chemical Formula 1 and at least one DNA damage inducing substance may be performed in any order. For example, the PRMT5 inhibitor of Chemical Formula 1 may be administered simultaneously, concomitantly, sequentially, continuously, alternatively, or separately with at least one co-administered DNA damage inducing substance.

In some embodiments, the present disclosure includes a method for treating and/or preventing a cancer in a human subject in need thereof, the method comprising administering to the human subject a therapeutically effective amount of a pharmaceutical combination which contains a PRMT5 inhibitor of Chemical Formula 1 and a DNA damage inducing substance as defined herein, to treat and/or prevent cancer in the human subject.

In some embodiments, the human subject may be a subject having multidrug resistance (MDR), or exhibiting resistance or poor responsiveness to DNA damage inducing substances, and the method comprises treating and/or preventing cancer in the subject.

In some embodiments, the human subject may be HRD (homologous recombination deficiency)-positive or HRD-negative, and the method comprises treating and/or preventing cancer in the subject.

In some embodiments, the present disclosure includes a method for treating and/or preventing cancer in a human subject having cancer, the method comprising administering to the human subject a therapeutically effective amount of a pharmaceutical combination which comprises a PRMT5 inhibitor of Chemical Formula 1 as defined herein and a DNA damage inducing substance, in combination with at least one pharmaceutically acceptable carrier and/or pharmaceutically acceptable diluent, to treat and/or prevent cancer in the human subject.

In some embodiments, the present disclosure includes a pharmaceutical combination for use in the treatment and/or prevention of cancer in a subject in need thereof, the pharmaceutical combination comprising a PRMT5 inhibitor of Chemical Formula 1 as defined herein and a DNA damage inducing substance.

In some embodiments, the present disclosure includes a pharmaceutical combination for use in the treatment of cancer in a subject in need thereof, the pharmaceutical combination comprising a PRMT5 inhibitor of Chemical Formula 1 as defined herein and a DNA damage inducing substance.

In some embodiments, the present disclosure includes a pharmaceutical combination for use in reducing tumor growth rate, reducing tumor size, or alleviating one or more symptoms associated with the presence of a tumor in a subject in need thereof, the pharmaceutical combination comprising a PRMT5 inhibitor of Chemical Formula 1 as defined herein and a DNA damage inducing substance.

In some embodiments, the present disclosure provides a pharmaceutical combination comprising a PRMT5 inhibitor of Chemical Formula 1 and a DNA damage inducing substance, which may be used for treating and/or preventing various types of cancer. According to another embodiment disclosed herein, the cancer includes, but is not limited to, one or more selected from the group consisting of acoustic neuroma, adenocarcinoma, adrenal cancer, anal cancer, angiosarcoma, benign monoclonal gammopathy, cholangiocarcinoma, bladder cancer, breast cancer, brain cancer, lymphoma, multiple myeloma, lacrimal gland tumor, bronchial cancer, cervical cancer, craniopharyngioma, colorectal cancer, epithelial carcinoma, ependymoma, adenoid cystic carcinoma (ACC), endothelial sarcoma, endometrial cancer, esophageal cancer, Barrett's adenocarcinoma, Ewing's sarcoma, eye cancer, gallbladder cancer, gastric cancer, colon cancer, gastrointestinal stromal tumor (GIST), head and neck cancer, oral cancer (OSCC), throat cancer, hematologic cancer, hemangioblastoma, inflammatory myofibroblastic tumor, immunocyte-associated amyloidosis, kidney cancer, renal cell carcinoma, liver cancer, lung cancer, melanoma, uveal melanoma, acute myeloid leukemia (AML), myelodysplastic syndrome (MDS), mesothelioma, myeloproliferative disorder (MPD), chronic idiopathic myelofibrosis, chronic myeloid leukemia (CML), chronic neutrophilic leukemia (CNL), neuroblastoma, neurofibroma, neuroendocrine cancer, osteosarcoma, ovarian cancer, papillary adenocarcinoma, pancreatic cancer, penile cancer, prostate cancer, rectal cancer, rhabdomyosarcoma, salivary gland cancer, skin cancer, small intestinal cancer, soft tissue sarcoma, thyroid cancer, urethral cancer, vaginal cancer, and vulvar cancer.

In some embodiments, the brain cancer includes, but is not limited to, one or more selected from the group consisting of meningioma, glioma, medulloblastoma, glioblastoma, and brain metastasis. The lung cancer also includes, but is not limited to, one or more selected from the group consisting of non-small cell lung cancer (NSCLC) and small cell lung cancer (SCLC).

In some embodiments, the breast cancer or ovarian cancer may be a BRCA1 wild-type cancer, a BRCA1 mutant cancer, a BRCA2 wild-type cancer, or a BRCA2 mutant cancer, or may be any one cancer selected from the group consisting of BRCA1 wild-type cancer and BRCA2 wild-type cancer, BRCA1 wild-type and BRCA2 mutant cancer, BRCA1 mutant and BRCA2 wild-type cancer, and BRCA1 mutant and BRCA2 mutant cancer. In a specific embodiment, the cancer may be HRD-positive or HRD-negative cancer.

### Definition

The general terms used in the chemical formulas may be defined as follows.

However, the meanings provided should not be construed as limiting the scope of the terms themselves. As used herein, the description of a numerical range for a variable is intended to indicate that the invention may be practiced with any value within the range for that variable. Accordingly, for inherently discrete variables, the variable may be equal to any integer value within the range, including the endpoints. Similarly, for inherently continuous variables, the variable may be equal to any real value within the range, including the endpoints. For example, a variable described as having values between 0 and 2 could be 0, 1, or 2, or for essentially discrete variables 0.0, 0.1, 0.01, 0.001, or any other real value for essentially continuous variables.

As used herein, the terms “treat,” “improve,” and “inhibit,” as well as words derived therefrom do not inherently imply 100% or complete treatment, improvement, or inhibition. Rather, there are various degrees of treatment, improvement, and inhibition that those skilled in the art would recognize as having potential benefit or therapeutic effects. In this regard, the disclosed methods can provide any amount of any level of treatment, improvement, or inhibition in a mammal. For example, a disorder, including a symptom or condition of the disorder, may be reduced by, for example, 100%, 90%, 80%, 70%, 60%, 50%, 40%, 30%, 20%, or 10%. Moreover, the treatment, improvement, or inhibition provided by the present methods may include treatment, improvement, or inhibition of one or more conditions or symptoms of a disorder, for example, cancer. Additionally, for purposes herein, “treatment,” “improvement,” or “inhibition” may encompass delaying the onset of a disorder, or a symptom or condition thereof.

As used herein, “prevention” refers to reducing or eliminating the likelihood of contracting a disease.

As used herein, the term “inhibitor” refers to a molecule that binds to an enzyme and partially or completely inhibits the activity of the enzyme.

As used herein, the term “pharmaceutical combinations” may include, in addition to the active compounds according to the present invention, other chemical components such as carriers, diluents, and excipients. Accordingly, the pharmaceutical combination may comprise, as needed, one or more pharmaceutically acceptable carriers, diluents, excipients, or combinations thereof. Such pharmaceutical combinations facilitate the administration of the active compound into a biological subject. Various techniques for administering pharmaceutical combinations comprising the compounds are known, including, but not limited to, oral, injectable, aerosol, parenteral, and topical administration. The pharmaceutical combination may be sterile and may further comprise additives such as preservatives, stabilizers, hydrating agents or emulsifying agents, salts and/or buffers for osmotic adjustment, and may additionally include other therapeutically useful substances, and may be formulated using conventional methods such as mixing, granulation, or coating.

As used herein, the term “carrier” refers to a compound that facilitates the delivery of another compound into a cell or tissue. For example, dimethyl sulfoxide (DMSO) is a conventional carrier that facilitates the delivery of many organic compounds into cells or tissues of a biological subject.

As used herein, a “diluent” is defined as a compound that not only stabilizes the biologically active form of the subject compound, but is also diluted in water to dissolve the compound. Salts dissolved in buffers are used as diluents in the art. A commonly used buffer is phosphate buffered saline, which mimics the salt form of human body fluids. Since buffer salts can control the pH of a solution at low concentrations, it is rare for a buffered diluent to alter the biological activity of a compound.

As used herein, the term “pharmaceutically acceptable” refers to a property that does not impair the biological activity or physicochemical properties of the compound.

### Combination

In some embodiments, the alkylating agent among the DNA damage inducing substances that can be used in combination with the PRMT5 inhibitor of Chemical Formula 1 may include or exclude cisplatin, carboplatin, oxaliplatin, mechlorethamine, cyclophosphamide, ifosfamide, melphalan, chlorambucil, carmustine, lomustine, nimustine, altretamine, busulfan, dacarbazine, procarbazine, temozolomide, thiotepa, lurbinectedin or any other agent.

In some embodiments, the antimetabolite among the DNA damage inducing substances that may be used in combination with the PRMT5 inhibitor of Chemical Formula 1 may include or exclude one or more of fluorouracil, capecitabine, cytarabine, gemcitabine, methotrexate, mercaptopurine, or any other agent.

In some embodiments, the topoisomerase inhibitor among the DNA damage inducing substances that may be used in combination with the PRMT5 inhibitor of Chemical Formula 1 may include or exclude one or more of etoposide, teniposide, topotecan, irinotecan, SN-38, dactinomycin, doxorubicin, daunorubicin, mitomycin, bleomycin, or any other agent.

In some embodiments, the microtubule inhibitor among the DNA damage inducing substances that may be used in combination with the PRMT5 inhibitor of Chemical Formula 1 may include, or exclude, one or more of vinblastine, vincristine, vinorelbine, paclitaxel, docetaxel, or any other agent.

In some embodiments, the poly(ADP-ribose) polymerase (PARP) inhibitor that may be used in combination with the PRMT5 inhibitor of Chemical Formula 1 may include or exclude one or more of olaparib, veliparib, niraparib, pamiparib, talazoparib, rucaparib, CEP-9722, E7016 (GPI 21016), fluzoparib, AZD5305, AZD9574, or any other agent.

When the PRMT5 inhibitor compound and the PARP inhibitor are used in combination, the content ratio of the PRMT5 inhibitor compound to the PARP inhibitor (PRMT5 inhibitor compound: PARP inhibitor) may be in a weight ratio ranging from 1:3000 to 1:0.00015, and may be used, for example, in a weight ratio ranging from 1:1000 to 1:0.001.

In one embodiment of the present invention, the pharmaceutical combination may be used in a form in which the PRMT5 inhibitor and the DNA damage inducing substance are administered simultaneously or at different times. For example, the PRMT5 inhibitor and the DNA damage inducing substance may be administered in the form of a single pharmaceutical composition.

Synergistic and/or Enhanced Combination

As used herein, the term “synergistic” refers to a therapeutic combination that is more effective than the additive effect of two or more single agents. Combination therapy provides “synergistic acting” and demonstrates “synergistic,” i.e., the effect achieved when the active ingredients are used together is greater than the sum of the effects resulting from the compounds used separately. A synergistic effect can be achieved when the active ingredients are: (1) co-formulated in a single unit dosage formulation and administered or delivered simultaneously; or (2) delivered as separate formulations in an alternative therapy. When delivered in alternative therapy, a synergistic effect can be achieved when the compounds are administered or delivered sequentially, for example, by different injections in separate syringes. Typically, during alterative therapy, effective doses of each active ingredient are administered sequentially, i.e., serially in time. In some embodiments, synergy is demonstrated by lower toxicity of the combination compared to the same dose of any single ingredient at the same total dosage. For example, when a PRMT5 inhibitor compound of Chemical Formula 1 is co-administered with a DNA damage inducing agent as described herein, the toxicity of the combination comprising the PRMT5 inhibitor compound as described herein and the DNA damage inducing agent a ratio of 50:50 (w/w) can be less than the toxicity of 100% (w/w) of PRMT5 inhibitor compound, and/or the toxicity of 100% (w/w) of DNA damage inducing agent, and wherein the combination has about the same level of efficacy or greater. Without wishing to be bound by theory, the inventors have recognized that while the PRMT5 inhibitor compound can be metabolized by a first set of metabolizing enzymes, the pharmaceutical combination comprising the DNA damage inducing agent, or a pharmaceutically acceptable salt thereof, can be metabolized by a second set of enzymes, such that the first set of enzymes and the second set of enzymes are not entirely identical. The result is that either the toxicity of the combination will be less than the toxicity of the single components of the combination, or the efficacy of the combination will be greater than the efficacy of the single components of the combination. The rationale for combining two or more anticancer therapies is not only to reduce toxicity and provide greater safety, but also to enhance efficacy beyond that provided by either agent alone. Increased efficacy is one of the benefits of combination therapies. The combinations are designed to target cellular signaling mechanisms so that multiple oncogenic pathways can be addressed concomitantly, resulting in enhanced anticancer activity and/or efficacy (e.g., tumor growth inhibition). The exemplary assays discussed herein demonstrate enhanced anticancer efficacy both in vitro and in vivo, clearly illustrating the advantage of combining Formula I with DNA damage inducing agents.

In measuring in vivo or therapeutic synergy, one measure of synergy is known as “Excess over Highest Single Agent” synergy. Excess over Highest Single Agent synergy occurs when a fixed dose combination is superior to the doses of both of its components, which is referred to as “Excess over Highest Single Agent.”

An effective amount of a compound (or a pharmaceutically acceptable salt thereof) may be understood to include an amount sufficient to prevent or inhibit the growth of tumor cells or the progression of cancer metastasis in the combination of the present invention. The therapeutic or pharmacological effectiveness of a dosage and administration regimen may also be characterized by its ability to induce, enhance, maintain or prolong remission in a patient experiencing a particular tumor.

### Method

In some embodiments of the present invention, there is provided an use of a pharmaceutical combination comprising a PRMT5 inhibitor of Chemical Formula 1 and a DNA damage inducing substance or pharmaceutically acceptable salts thereof, for the manufacture of a medicament for the treatment of a disease or condition in which inhibition of PRMT5 protein is ineffective.

In some embodiments, the present disclosure provides a method of treating and/or preventing cancer in a human subject in need thereof, the method comprising administering to the human subject a pharmaceutical combination comprising a PRMT5 inhibitor of formula I and a DNA damage inducing agent, or a pharmaceutically acceptable salt thereof. Cancers may include at least one selected the group consisting of acoustic neuroma, adenocarcinoma, adrenal cancer, anal cancer, angiosarcoma, benign monoclonal gammopathy, cholangiocarcinoma, bladder cancer, breast cancer, brain cancer (meningioma, glioma, medulloblastoma, glioblastoma, and brain metastases), lymphoma, multiple myeloma, lacrimal gland tumor, bronchial cancer, cervical cancer, craniopharyngioma, colorectal cancer, epithelial carcinoma, ependymoma, adenoid cystic carcinoma (ACC), endothelial sarcoma, endometrial cancer, esophageal cancer, Barrett's adenocarcinoma, Ewing's sarcoma, eye cancer, gallbladder cancer, gastric cancer, colon cancer, gastrointestinal stromal tumor (GIST), head and neck cancer, oral cancer (OSCC), throat cancer, hematologic cancer, hemangioblastoma, inflammatory myofibroblastic tumor, immunocyte-associated amyloidosis, kidney cancer, renal cell carcinoma, liver cancer, lung cancer, melanoma, uveal melanoma, acute myeloid leukemia (AML), myelodysplastic syndrome (MDS), mesothelioma, myeloproliferative disorder (MPD), chronic idiopathic myelofibrosis, chronic myeloid leukemia (CML), chronic neutrophilic leukemia (CNL), neuroblastoma, neurofibroma, neuroendocrine cancer, osteosarcoma, ovarian cancer, papillary adenocarcinoma, pancreatic cancer, penile cancer, prostate cancer, rectal cancer, rhabdomyosarcoma, salivary gland cancer, skin cancer, small intestinal cancer, soft tissue sarcoma, thyroid cancer, urethral cancer, vaginal cancer, and vulvar cancer.

In some embodiments, the brain cancer may be one or more selected from the group consisting of meningioma, glioma, medulloblastoma, glioblastoma, and brain metastatic cancer, and the lung cancer may be non-small cell lung cancer (NSCLC) or small cell lung cancer (SCLC).

The breast cancer and/or ovarian cancer may be a BRCA1 wild-type cancer, a BRCA1 mutant cancer, a BRCA2 wild-type cancer, or a BRCA2 mutant cancer, or may be one selected from the group consisting of BRCA1 wild-type and BRCA2 wild-type cancer, BRCA1 wild-type and BRCA2 mutant cancer, BRCA1 mutant and BRCA2 wild-type cancer, and BRCA1 mutant and BRCA2 mutant cancer. In a specific embodiment, the cancer may be HRD-positive or HRD-negative cancer.

In some embodiments, the present invention provides a method for treating and/or preventing cancer in a subject in need thereof. In some embodiments, the present invention provides a method for reducing tumor growth rate, reducing tumor size, alleviating one or more symptoms associated with the presence of a tumor, or eliminating the tumor in a subject in need thereof. The cancer may include or exclude solid tumors, leukemia, or lymphoma.

### Administration

In some embodiments, the pharmaceutical combination provided herein may include a pharmaceutical combination comprising a PRMT5 inhibitor of Chemical Formula 1, a DNA damage inducing substance or pharmaceutically acceptable salts thereof, which is administered by any route appropriate to the condition to be treated. Suitable routes may include or exclude one or more selected from the group consisting of oral, parenteral, subcutaneous, intramuscular, intravenous, intraarterial, intradermal, intrathecal, and epidural), intraperitoneal (IP), transdermal, rectal, intranasal, topical, buccal, and sublingual), vaginal, intrapulmonary, and intranasal. In some embodiments, for local treatment, the compound is administered by intratumoral administration, including by perfusing or otherwise contacting the tumor with an inhibitor. The preferred route may vary depending upon, for example, the condition of the recipient/subject. In some embodiments, if the compound is administered orally, it may be formulated as a pill, capsule, tablet, or the like, together with pharmaceutically acceptable carriers and/or excipients. In some embodiments, if the compound is administered parenterally, it may be formulated together with a pharmaceutically acceptable parenteral vehicle and in a unit dosage injectable form as described herein.

An appropriate dosage and dosing regimen may be determined using a range-finding approach, which may be a function of factors such as subject weight, body volume, body surface area, site of administration, route of administration, and the cancer prognosis of the subject. Typically, the treatment is initiated with a lower dose than the optimal dose of the combination of the present invention. Thereafter, the dosage may be incrementally increased until the optimal therapeutic effect is achieved under the given circumstances. The method may involve administering from about 0.01 g to about 200 mg of at least one compound of the present invention per kilogram of body weight of the subject. For example, for a 70 kg patient, a dosage of from about 1 g to about 1000 mg of the compound may be used, depending on the physiological response of the patient.

In some embodiments, the dosage of the pharmaceutically active agents described herein for use in the treatment and/or prevention of cancer as described in the present disclosure, may be from about 0.001 to about 1 mg per kilogram of body weight of subject per day. For example, the dosage may be about 0.001 mg, 0.002 mg, 0.005 mg, 0.010 mg, 0.015 mg, 0.020 mg, 0.025 mg, 0.050 mg, 0.075 mg, 0.1 mg, 0.15 mg, 0.2 mg, 0.25 mg, 0.5 mg, 0.75 mg, or 1 mg/kg body weight/day. The dosage of the pharmaceutically active agent(s) described herein for use in the methods disclosed may also be from about 1 to about 1000 mg per kilogram of body weight per day. For example, the dosage may be about 1 mg, 2 mg, 5 mg, 10 mg, 15 mg, 20 mg, 25 mg, 50 mg, 75 mg, 100 mg, 150 mg, 200 mg, 250 mg, 500 mg, 750 mg, or 1000 mg/kg body weight/day.

In some embodiments, in combination with a DNA damage inducing substance administered at a dosage of about 1 mg/kg/day, about 2 mg/kg/day, about 5 mg/kg/day, about 10 mg/kg/day, about 15 mg/kg/day, about 20 mg/kg/day, about 25 mg/kg/day, about 30 mg/kg/day, about 35 mg/kg/day, about 40 mg/kg/day, about 45 mg/kg/day, about 50 mg/kg/day, about 60 mg/kg/day, about 70 mg/kg/day, about 80 mg/kg/day, about 90 mg/kg/day, about 100 mg/kg/day, about 125 mg/kg/day, about 150 mg/kg/day, about 175 mg/kg/day, about 200 mg/kg/day, about 250 mg/kg/day, or about 300 mg/kg/day, the PRMT5 inhibitor of Chemical Formula 1 in a pharmaceutical combination for treating and/or preventing a disease or condition as described herein (for example, treating and/or preventing cancer as disclosed herein), may be administered at a dosage of about 1 mg/kg/day, about 2 mg/kg/day, about 5 mg/kg/day, about 10 mg/kg/day, about 15 mg/kg/day, about 20 mg/kg/day, about 25 mg/kg/day, about 30 mg/kg/day, about 35 mg/kg/day, about 40 mg/kg/day, about 45 mg/kg/day, about 50 mg/kg/day, about 60 mg/kg/day, about 70 mg/kg/day, about 80 mg/kg/day, about 90 mg/kg/day, about 100 mg/kg/day, about 125 mg/kg/day, about 150 mg/kg/day, about 175 mg/kg/day, about 200 mg/kg/day, about 250 mg/kg/day, or about 300 mg/kg/day.

In some embodiments, the pharmaceutical combination of the present disclosure may be administered systemically, for example via intravenous, intraarterial, or intraperitoneal administration. Accordingly, the resulting circulating concentration of the PRMT5 inhibitor compound of Chemical Formula 1 may range from approximately 0.001 micromolar (μM) to approximately 150 μM, or even higher, including about 200 μM, 300 μM, 400 μM, 500 μM, 600 μM, 700 μM, 800 μM, 900 μM, or 1000 μM. The final circulating concentration may be any one of the following (all expressed in micromolar): 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, or 150 μM, or any value between any two of the aforementioned values, or any higher concentration, or any concentration within the stated range. An ordinary person skilled in the art will appreciate the appropriate dosage of one or more active agents, taking into consideration the properties and principles of the formulation as previously discussed herein.

In some embodiments, the present disclosure provides a method of administration wherein a pharmaceutical combination comprising a PRMT5 inhibitor of Chemical Formula 1, a DNA damage inducing substance, and/or pharmaceutically acceptable salts thereof is administered simultaneously, concomitantly, sequentially, continuously, alternately, or separately.

In a pharmaceutical combination comprising a PRMT5 inhibitor of Chemical Formula 1 or a pharmaceutically acceptable salt thereof and a DNA damage inducing substance, the co-administration of the PRMT5 inhibitor and at least one DNA damage inducing substance may provide enhanced relief or improvement of one or more symptoms, a reduction in the length or extent of the disease, a delay or retardation in disease progression, an improvement, mitigation, or stabilization of the disease condition, partial or complete remission, extended survival, and/or other beneficial therapeutic outcomes. Such treatment may involve administration either simultaneously or sequentially in any or order during the course of treatment. One of ordinary skill in the art will readily understand how to administer the formulation or therapy simultaneously, alternately, or sequentially between doses and over appropriate time intervals. When the PRMT5 inhibitor compound is administered at a different time than the DNA damage inducing substance as described herein, the administration may be separated by an interval of, for example, 30 minutes, 1 hour, 2 hours, 3 hours, 4 hours, 5 hours, 6 hours, 7 hours, 8 hours, 9 hours, 10 hours, 11 hours, 1 day, 2 days, 3 days, 4 days, 5 days, 6 days, 1 week, 2 weeks, 3 weeks, or 1 month, or any time interval between any two of the aforementioned periods. Dosage frequency may include QD (once daily), BID (twice daily), TID (three times daily), QID (four times daily), or weekly regimens such as QIW (once weekly), BIW (twice weekly), or QW (weekly). Dosages may also be administered PRN (as needed) or at bedtime (hora somni).

In some embodiments, the treatment according to the present invention may involve administering to a human subject a pharmaceutical combination comprising a PRMT5 inhibitor of Chemical Formula 1 and a DNA damage inducing substance or a pharmaceutically acceptable salt thereof. For example, the pharmaceutical combination disclosed herein may be used in the manufacture of a medicament to promote general well-being in the subject or to reduce one or more side effects of cancer therapy. An ordinary person skilled in the art will readily understand that various formulations may be beneficially administered depending, for example, on the specific disease to be treated. In some embodiments, in which at least one DNA damage inducing substance selected from a pharmaceutical combination comprising a PRMT5 inhibitor of Chemical Formula 1, a DNA damage inducing substance, or a pharmaceutically acceptable salt thereof, is co-administered initially, the administration of at least one DNA damage inducing substance selected from the pharmaceutical combination comprising a PRMT5 inhibitor of Chemical Formula 1, a DNA damage inducing substance, or a pharmaceutically acceptable salt thereof, may be continued thereafter.

The administration of a pharmaceutical combination comprising a PRMT5 inhibitor of Chemical Formula 1 and a DNA damage inducing substance or a pharmaceutically acceptable salt thereof, may occur at any time during the course of a disease or disorder, or before or after the onset of one or more symptoms of such disease or disorder. In one embodiment, at least one DNA damage inducing substance selected from the pharmaceutical combination comprising a PRMT5 inhibitor of Chemical Formula 1, a DNA damage inducing substance or a pharmaceutically acceptable salt thereof, is administered on a daily basis for an extended period to assist in the ongoing management of symptoms. In another embodiment, the DNA damage inducing substance selected from the pharmaceutical combination comprising a PRMT5 inhibitor of Chemical Formula 1, a DNA damage inducing substance, or a pharmaceutically acceptable salt thereof, is administered on a daily basis for an extended period to prevent or delay the onset of the disease or disorder.

### Effect of Invention

The present invention provides an use of a combination therapy comprising a PRMT5 inhibitor and various anticancer agents to overcome resistance to anticancer agents, and ultimately to treat, ameliorate, or prevent cancer.

### BRIEF DESCRIPTION OF THE DRAWINGS

The drawings form a part of the present specification and are included to further illustrate certain embodiments of the invention described herein. These embodiments are better understood when read in conjunction with one or more of the drawings referenced below and the detailed description provided herein.

FIG. 1 illustrates the combined treatment effect of a representative combination of a PRMT5 inhibitor according to an embodiment of the present invention and a DNA damage inducing substance, specifically an alkylating agent (cisplatin) in the A549 lung cancer cell line, in terms of cell survival rate and a synergistic effect scale (Bliss score).

FIG. 2 illustrates the combined treatment effect of a representative combination of a PRMT5 inhibitor according to an embodiment of the present invention and a DNA damage inducing substance, specifically an alkylating agent (cisplatin), in the cisplatin-resistant A549 lung cancer cell line (A549/CR, cisplatin-resistance), in terms of cell survival rate and a synergistic effect scale (Bliss score).

FIG. 3 illustrates the combined treatment effect of a representative combination of a PRMT5 inhibitor according to an embodiment of the present invention and a DNA damage inducing substance, specifically an alkylating agent (cisplatin), in the paclitaxel-resistant A549 lung cancer cell line (A549/PR, paclitaxel-resistance), in terms of cell survival rate and a synergistic effect scale (Bliss score).

FIG. 4 illustrates the combined treatment effect of a representative combination of a PRMT5 inhibitor according to an embodiment of the present invention and a DNA damage inducing substance, specifically an alkylating agent (cisplatin), in the NCI-H1048 small cell lung cancer cell line, in terms of cell survival rate and a synergistic effect scale (Bliss score).

FIG. 5 illustrates the combined treatment effect of a representative combination of a PRMT5 inhibitor according to an embodiment of the present invention and a DNA damage inducing substance, specifically an alkylating agent (temozolomide), in the U-87 MG glioblastoma cell line, in terms of cell survival rate and a synergistic effect scale (Bliss score).

FIG. 6 illustrates the combined treatment effect of a representative combination of a PRMT5 inhibitor according to an embodiment of the present invention and a DNA damage inducing substance, specifically an alkylating agent (lurbinectedin), in the NCI-H1048 small cell lung cancer cell line, in terms of cell survival rate and a synergistic effect scale (Bliss score).

FIG. 7 illustrates the combined treatment effect of a representative combination of a PRMT5 inhibitor according to an embodiment of the present invention and a DNA damage inducing substance, specifically an anti-metabolite (gemcitabine), in the CAPAN-1 pancreatic cancer cell line, in terms of cell survival rate and a synergistic effect scale (Bliss score).

FIG. 8 illustrates the combined treatment effect of a representative combination of a PRMT5 inhibitor according to an embodiment of the present invention and a DNA damage inducing substance, specifically a topoisomerase inhibitor (etoposide), in the NCI-H1048 small cell lung cancer cell line, in terms of cell survival rate and a synergistic effect scale (Bliss score).

FIG. 9 illustrates the combined treatment effect of a representative combination of a PRMT5 inhibitor according to an embodiment of the present invention and a DNA damage inducing substance, specifically a topoisomerase inhibitor (SN-38), in the CAPAN-1 pancreatic cancer cell line, in terms of cell survival rate and a synergistic effect scale (Bliss score).

FIG. 10 illustrates the combined treatment effect of a representative combination of a PRMT5 inhibitor according to an embodiment of the present invention and a DNA damage inducing substance, specifically a topoisomerase inhibitor (SN-38), in the Mia PaCa-2 pancreatic cancer cell line, in terms of cell survival rate and a synergistic effect scale (Bliss score).

FIG. 11 illustrates the combined treatment effect of a representative combination of a PRMT5 inhibitor according to an embodiment of the present invention and a DNA damage inducing substance, specifically a microtubule inhibitor (paclitaxel), in the MDA-MB-231 triple-negative breast cancer cell line, in terms of cell survival rate and a synergistic effect scale (Bliss score).

FIG. 12 illustrates the combined treatment effect of a representative combination of a PRMT5 inhibitor according to an embodiment of the present invention and a PARP inhibitor (olaparib), in the SNU-324 pancreatic cancer cell line harboring a BRCA2 mutation, in terms of cell survival rate and a synergistic effect scale (Bliss score).

FIG. 13 illustrates the combined treatment effect of a representative combination of a PRMT5 inhibitor according to an embodiment of the present invention and a PARP inhibitor (olaparib), in the Mia PaCa-2 pancreatic cancer cell line with wild-type BRCA2, in terms of cell survival rate and a synergistic effect scale (Bliss score).

FIG. 14 illustrates the combined treatment effect of a representative combination of a PRMT5 inhibitor according to an embodiment of the present invention and a PARP inhibitor (olaparib), in the CAPAN-1 pancreatic cancer cell line (BRCA2 mutation), in terms of cell survival rate and a synergistic effect scale (Bliss score).

FIG. 15 illustrates the combined treatment effect of a representative combination of a PRMT5 inhibitor according to an embodiment of the present invention and a PARP inhibitor (olaparib), in the PANC-1 pancreatic cancer cell line (wild-type BRCA1/BRCA2), in terms of cell survival rate and a synergistic effect scale (Bliss score).

FIG. 16 illustrates the combined treatment effect of a representative combination of a PRMT5 inhibitor according to an embodiment of the present invention and a PARP inhibitor (olaparib), in the OVCAR3 cell line, in terms of cell survival rate and a synergistic effect scale (Bliss score).

FIG. 17 illustrates the combined treatment effect of a representative combination of a PRMT5 inhibitor according to an embodiment of the present invention and a PARP inhibitor (olaparib), in the PA-1 cell line, in terms of cell survival rate and a synergistic effect scale (Bliss score).

FIG. 18 illustrates the combined treatment effect of a representative combination of a PRMT5 inhibitor according to an embodiment of the present invention and a PARP inhibitor (olaparib), in the MDA-MB-231 cell line, in terms of cell survival rate and a synergistic effect scale (Bliss score).

FIG. 19 illustrates the combined treatment effect of a representative combination of a PRMT5 inhibitor according to an embodiment of the present invention and a PARP inhibitor (olaparib), in the NCI-H1048 cell line, in terms of cell survival rate and a synergistic effect scale (Bliss score).

FIG. 20 illustrates the combined treatment effect of a representative combination of a PRMT5 inhibitor according to an embodiment of the present invention and a PARP inhibitor (AZD2461), in the Mia-Paca-1 cell line, in terms of cell survival rate and a synergistic effect scale (Bliss score).

FIG. 21 illustrates the combined treatment effect of a representative combination of a PRMT5 inhibitor according to an embodiment of the present invention and a PARP inhibitor (AZD2461), in the CAPAN-1 cell line, in terms of cell survival rate and a synergistic effect scale (Bliss score).

FIG. 22 illustrates the combined treatment effect of a representative combination of a PRMT5 inhibitor and a PARP inhibitor (AZD2461), in the PANC-1 cell line, in terms of cell survival rate and a synergistic effect scale (Bliss score).

FIG. 23 illustrates the combined treatment effect of a representative combination of a PRMT5 inhibitor and a PARP inhibitor (E7016), in the Mia-Paca-2 cell line, in terms of cell survival rate and a synergistic effect scale (Bliss score).

FIG. 24 illustrates the combined treatment effect of a representative combination of a PRMT5 inhibitor and a PARP inhibitor (E7016), in the CAPAN-1 cell line, in terms of cell survival rate and a synergistic effect scale (Bliss score).

FIG. 25 illustrates the combined treatment effect of a representative combination of a PRMT5 inhibitor and a PARP inhibitor (E7016), in the PANC-1 cell line, in terms of cell survival rate and a synergistic effect scale (Bliss score).

FIG. 26 illustrates the tumor growth inhibition resulting from monotherapy with a PRMT5 inhibitor or combination therapy with cisplatin in a subcutaneous xenograft mouse model using NCI-H1048 small cell lung cancer cells.

FIG. 27 illustrates the tumor growth inhibition resulting from monotherapy with a PRMT5 inhibitor or combination therapy with gemcitabine in a subcutaneous xenograft mouse model using human CAPAN-1 pancreatic cancer cells.

FIG. 28 illustrates the tumor growth inhibition resulting from monotherapy with a PRMT5 inhibitor or combination therapy with gemcitabine in a subcutaneous xenograft mouse model using Mia PaCa-2 human pancreatic cancer cells.

FIG. 29 illustrates the tumor growth inhibition resulting from monotherapy with a PRMT5 inhibitor or combination therapy with paclitaxel in a subcutaneous xenograft mouse model of CTG-1932 patient-derived non-small cell lung cancer (NSCLC) with low responsiveness to DNA damage inducing substances.

FIG. 30 visualizes gene expression changes following treatment with a PRMT5 inhibitor in a patient-derived GTG-2393 lung cancer xenograft mouse model, using Z-scores of log 2FPKM values in the form of a heat map.

### MODE FOR INVENTION

The following examples are provided to illustrate, but not limit, the methods described herein. Various modifications may be made by those skilled in the art without departing from the true spirit and scope of the subject matter described herein.

### Example 1. Preparation of PRMT5 Inhibitor

### 1-1: Materials

The PRMT5 inhibitor compounds of the present disclosure are described in PCT Publication No. WO2021066578 and can be made and characterized by the methods described herein. As a representative example, the synthesis of Compound-87, Compound-112 or Compound-271 is as described below:

### 1-2: Compound-87

4-[(2R)-3-(3,4-dihydro-1H-isoquinolin-2-yl)-2-hydroxy-propyl]-8-[(1-methyl-4-piperidyl)oxy]-2,3-dihydro-1,4-benzoxazepin-5-one

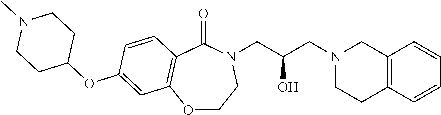 [SMILES: CN1CCC(Oc2ccc3c(c2)OCCN(C[C@H](O)CN2CCc4ccccc4C2)C3=O)CC1]

4-[(2R)-3-(3,4-dihydro-1H-isoquinolin-2-yl)-2-hydroxy-propyl]-8-(4-piperidyloxy)-2,3-dihydro-1,4-benzoxazepin-5-one 2 hydrochloride (100 mg, 0.19 mmol) (mmol) (prepared according to the procedure given in page 67, lines 639-640 of WO2021066578) was dissolved in methanol, and were added with paraformaldehyde (57 mg, 1.9 mmol) and sodium cyanoborohydride (36 mg, 0.57 mmol). The reaction solution was stirred at room temperature until the reaction was complete. Saturated ammonium chloride aqueous solution was added, stirred for 30 minutes, and then 1 N sodium hydroxide aqueous solution was added to basify the solution. Ethyl acetate was added to the mixture, extracted three times, and dried over anhydrous sodium sulfate. The solvent was removed by reduced pressure evaporation, and the pale yellow oily liquid obtained was purified by rapid chromatography to synthesize the title compound as a white solid.

1H NMR (400 MHz, Methanol-d4) δ 7.66 (d, J=8.8 Hz, 1H), 7.16-7.00 (m, 4H), 6.76 (dd, J=8.8, 2.4 Hz, 1H), 6.58 (d, J=2.4 Hz, 1H), 4.56-4.41 (m, 3H), 4.27-4.16 (m, 1H), 3.97 (dd, J=13.9, 3.5 Hz, 1H), 3.79-3.63 (m, 4H), 3.41 (dd, J=13.9, 7.7 Hz, 1H), 2.98-2.88 (m, 2H), 2.90-2.80 (m, 2H), 2.79-2.68 (m, 2H), 2.68-2.56 (m, 2H), 2.49-2.36 (m, 2H), 2.32 (s, 3H), 2.10-2.01 (m, 3H), 1.90-1.76 (m, 2H).

### 1-3: Compound-112

4-[(2R)-3-(3,4-dihydro-1H-isoquinolin-2-yl)-2-hydroxy-propyl]-2,2-dimethyl-8-[(1-methyl-4-piperidyl)oxy]-3H-1,4-benzoxazepin-5-one

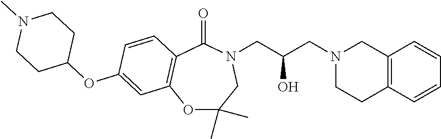 [SMILES: CN1CCC(Oc2ccc3c(c2)OC(C)(C)CN(C[C@H](O)CN2CCc4ccccc4C2)C3=O)CC1]

4-[(2R)-3-(3,4-dihydro-1H-isoquinolin-2-yl)-2-hydroxy-propyl]-2,2-dimethyl-8-(4-piperidyloxy)-3H-1,4-benzoxazepin-5-one 2 hydrochloride

The title compound was synthesized by the same method as Example 87, using the material obtained from page 84, line 853 to page 85, line 866 of WO2021066578 as a starting material.

1H NMR (400 MHz, Methanol-d4) δ 7.57 (d, J=8.6 Hz, 1H), 7.17-7.02 (m, 4H), 6.81 (d, J=8.7 Hz, 1H), 6.52 (s, 1H), 4.56-4.46 (m, 1H), 4.33-4.21 (m, 1H), 4.03 (d, J=13.3 Hz, 1H), 3.75 (s, 2H), 3.50 (s, 2H), 3.40 (dd, J=14.0, 8.1 Hz, 1H), 2.98-2.81 (m, 4H), 2.80-2.66 (m, 2H), 2.66-2.54 (m, 2H), 2.51-2.39 (m, 2H), 2.34 (s, 3H), 2.10-1.98 (m, 2H), 1.89-1.76 (m, 2H), 1.43 (s, 3H), 1.34 (s, 3H).

### 1-4: Compound-271

4-[(2R)-3-(3,4-dihydro-1H-isoquinolin-2-yl)-2-hydroxy-propyl]-8-[(3R)-1-(oxetan-3-yl)pyrrolidin-3-yl]oxy-2,3-dihydro-1,4-benzoxazepin-5-one

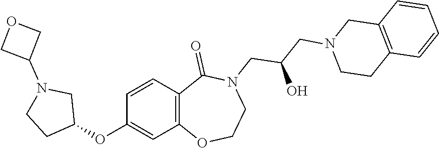 [SMILES: O=C1c2ccc(O[C@@H]3CCN(C4COC4)C3)cc2OCCN1C[C@H](O)CN1CCc2ccccc2C1]

The title compound was synthesized by the same method as in Example 87 using 4-[(2R)-3-(3,4-dihydro-1H-isoquinolin-2-yl)-2-hydroxy-propyl]-8-[(3R)-pyrrolidin-3-yl]oxy-2,3-dihydro-1,4-benzoxazepin-5-one 2 hydrochloride as a starting material and substituting paraformaldehyde with oxetan-3-one.

1H NMR (400 MHz, Methanol-d4) δ 7.68 (d, J=8.8 Hz, 1H), 7.16-7.02 (m, 4H), 6.73 (dd, J=8.8, 2.5 Hz, 1H), 6.54 (d, J=2.4 Hz, 1H), 4.98 (td, J=6.1, 5.3, 2.8 Hz, 1H), 4.75 (t, J=6.7 Hz, 2H), 4.64 (dt, J=11.8, 6.1 Hz, 2H), 4.48 (t, J=4.9 Hz, 2H), 4.23 (qd, J=7.2, 3.7 Hz, 1H), 3.98 (dd, J 13.9, 3.6 Hz, 1H), 3.80-3.71 (m, 5H), 3.42 (dd, J 13.9, 7.7 Hz, 1H), 2.98-2.80 (m, 7H), 2.69-2.62 (m, 2H), 2.56 (ddd, J=9.2, 7.8, 6.1 Hz, 1H), 2.38 (dq, J=14.0, 7.1 Hz, 1H), 2.01 (dt, J=13.7, 4.4 Hz, 1H).

### Example 2: In Vitro Data

### 2-1: Cell Culture

Most cell lines were obtained from the American Type Culture Collection (ATCC) or the Korean Cell Line Bank (KCLB). Combination experiments in cisplatin-resistant and paclitaxel-resistant cell lines were performed at the Asan Preclinical Evaluation center for cancer therapeutiX of Asan Medical Center, Seoul. All cell lines were maintained in appropriate growth medium for the cell line in 100-pi dishes oRT75 (75 cm2) flasks, cultured at 37° C. with 5% CO₂, and passaged every 3-4 days. Growth medium conditions and cell line information are listed in Table 2, and the cells were counted using Countess (Invitrogen) to obtain the number of viable cells. Mutation and HRD status by cell type are described in Table 3.

TABLE 2

| Cell line | Cancer type | Growth medium conditions |
|---|---|---|
| A549 | Lung cancer (non-small | RPMI1640 + 10% heat inactivated FBS + |
| | cell lung cancer) | 1% PS |
| A549/CR | Lung cancer (non-small | RPMI1640 + 10% heat inactivated FBS + |
| | cell lung cancer) | 1% PS |
| A549/PR | Lung cancer (non-small | RPMI1640 + 10% heat inactivated FBS + |
| | cell lung cancer) | 1% PS |
| CAPAN-1 | Pancreatic cancer | RPMI1640 + 10% heat inactivated FBS + |
| | | 1% PS |
| NCI-H1048 | Lung cancer (small cell | DMEM:F12 + 5 μg/ml insulin + 10 μg/ml |
| | lung cancer) | transferrin + 30 nM sodium selenite + 10 nM |
| | | hydrocortisone + 10 nM β-estradiol + 2 mM L- |
| | | glutamine + 5% heat inactivated FBS + 1% PS |
| MDA-MB-231 | Breast cancer | RPMI1640 + 10% heat inactivated FBS + |
| | | 1% PS |
| Mia PaCa-2 | Pancreatic cancer | DMEM + 10% heat inactivated FBS + 1% PS |
| SNU-324 | Pancreatic cancer | RPMI1640 + 10% heat inactivated FBS + |
| | | 1% PS |
| OVCAR3 | Ovarian cancer | RPMI1640 + 2 mM L-alanyl-L-glutamine + |
| | | 1 mM Na pyruvate + 10% heat inactivated |
| | | FBS + 1% PS |
| PA-1 | Ovarian cancer | RPMI1640 + 2 mM L-alanyl-L-glutamine + |
| | | 1 mM Na pyruvate + 10% heat inactivated |
| | | FBS + 1% PS |
| PANC-1 | Pancreatic cancer | DMEM + 10% heat inactivated FBS + 1% PS |
| U-87 MG | Brain cancer | MEM + 10% heat inactivated FBS + 1% PS |
| | (glioblastoma) | |

In the Table 2, A549/CR: cisplatin-resistance A549 cell, A549/PR: paclitaxel-resistance A549 cell, FBS: fetal bovine serum, RPMI-1640: Roswell Park Memorial Institute 1640 medium, DMEM: Dulbecco's Modified Eagle Medium, Han's F-12, P/S: penicillin/streptomycin.

TABLE 3

| Cell line | Mutation | HRD Status |
|---|---|---|
| CAPAN-1 | BRCA1 amp | HRD-positive |
| | BRCA2 mut | |
| MDA-MB-231 | BRCA1 WT, | HRD-negative |
| | BRCA WT | |
| SNU-324 | BRCA1 WT, | HRD-positive |
| | BRCA2 mut | |
| OVCA3 | BRCA1 WT, | HRD-negative |
| | BRCA2 WT | |
| PA-1 | BRCA1 WT, | HRD-negative |
| | BRCA2 WT | |
| Mia PaCa-2 | BRCA1 amp | HRD-negative |
| | BRCA2 WT | |
| PANC-1 | BRCA1 WT, | HRD-negative |
| | BRCA2 WT | |
| NCI-H1048 | BRCA1 WT, | HRD-negative |
| | BRCA2 WT | |

In Table 3, HRD: Homologous Recombination Deficiency, amp: amplification, mut: mutation, WT: Wild type.

### 2-2: Cell Viability Assay

As shown in Table 2, 100 to 1,500 cells of various cells were dispensed into each well of a 96-well or 384-well plate. The next day, compound-87 was diluted 1/2 to 1/4 to a concentration of 20 μM to 0.002 μM and treated at a set concentration range, and the co-administered drug was diluted 1/2 to 1/3 to a concentration of 100 μM to 0.00046 μM and treated at a 1:1 (v/v) ratio. Finally, the treatment ratio of compound-87 and the co-administered substance was between 1:20000 and 1:0.00015, and in clinical practice, compound-87: drug to be co-administered can be treated at a ratio of 1:10000 to 1:0.0001.

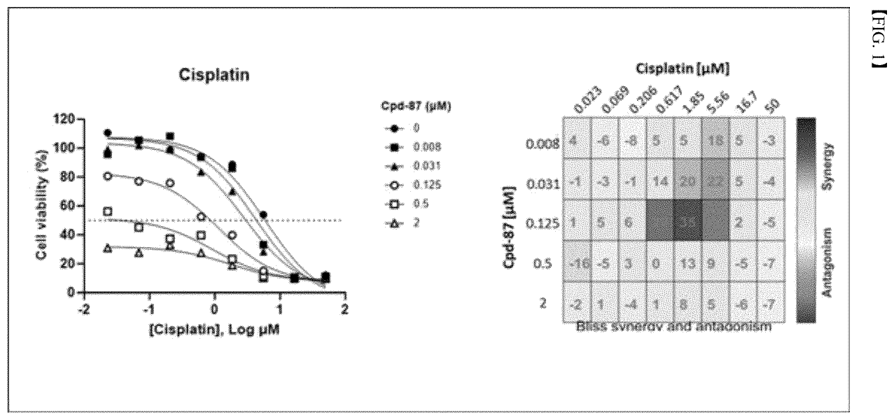

A549 non-small cell lung cancer cell line was treated with compound-87 at concentration of 0.008, 0.031, 0.125, 0.5, or 2 μM and cisplatin, an alkylating agent as a co-administered agent, at concentration of 0.023, 0.069, 0.206, 0.617, 1.85, 5.56, 16.7, or 50 μM (FIG. 1).

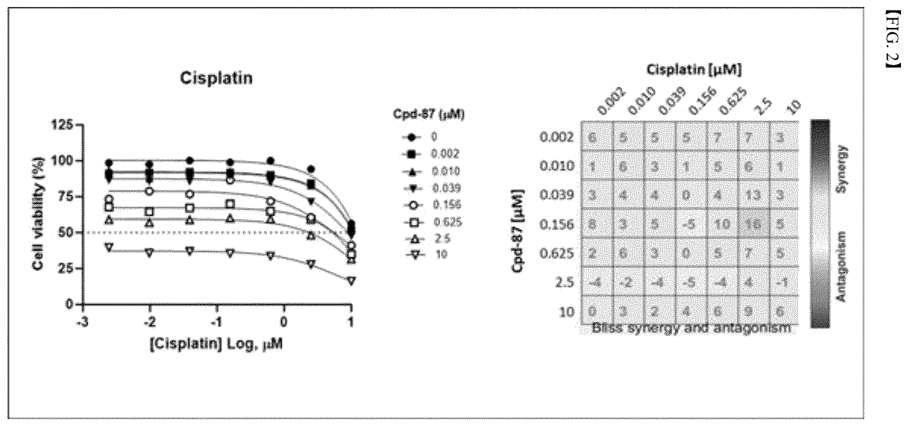

A549/CR cisplatin-resistant non-small cell lung cancer cell line was treated with compound-87 at concentrations of 0.002, 0.010, 0.039, 0.156, 0.625, 2.5, or 10 PM, and cisplatin as a co-administered agent was treated at concentration of 0.002, 0.010, 0.039, 0.156, 0.625, 2.5, or 10 μM (FIG. 2).

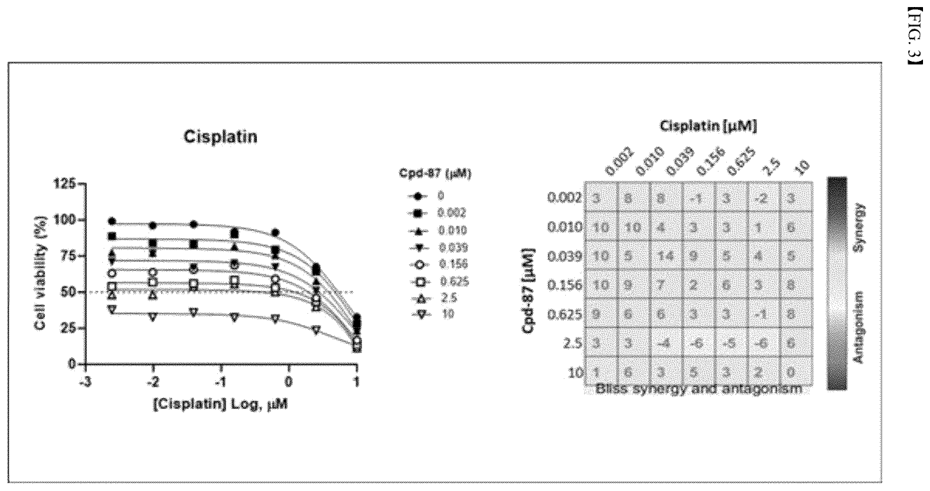

A549/PR paclitaxel-resistant non-small cell lung cancer cell line was treated with compound-87 at concentrations of 0.002, 0.010, 0.039, 0.156, 0.625, 2.5, or 10 PM, and cisplatin as a co-administered agent was treated at concentration of 0.002, 0.010, 0.039, 0.156, 0.625, 2.5, or 10 μM (FIG. 3).

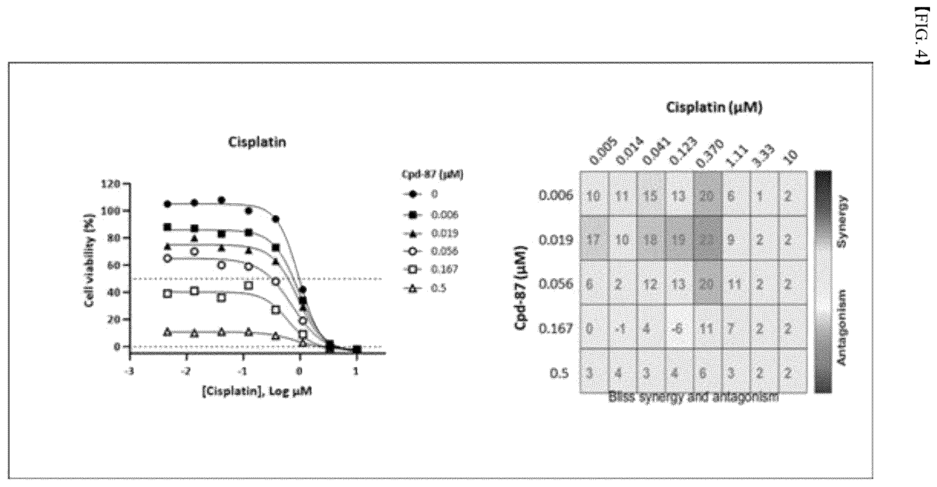

NCI-H1048 small cell lung cancer cell line was treated with compound-87 at concentrations of 0.006, 0.019, 0.056, 0.167, or 0.5 μM, and cisplatin as a co-administered agent was treated at concentrations of 0.005, 0.014, 0.041, 0.123, 0.037, 1.11, 3.33, or 10 M (FIG. 4).

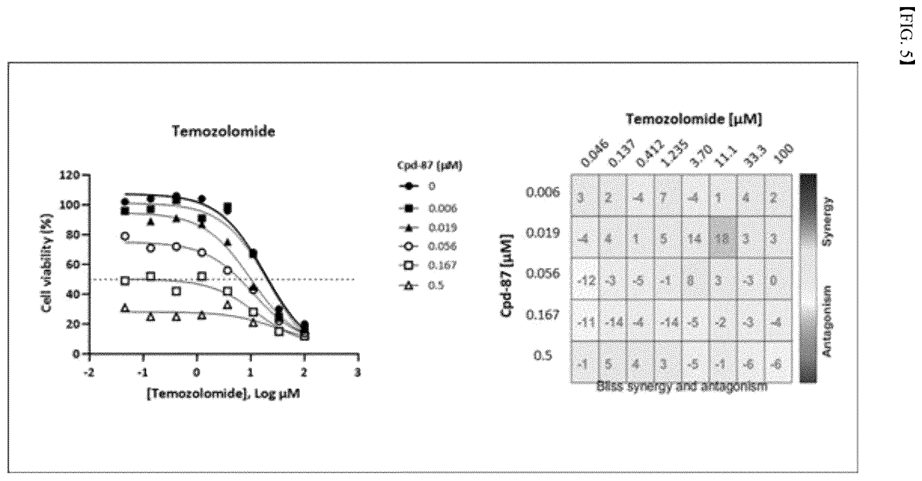

U-87 MG brain cancer cell line was treated with compound-87 at concentrations of 0.006, 0.019, 0.056, 0.167, or 0.5 μM, and temozolomide, an alkylating agent, as a co-administered agent, at concentration of 0.046, 0.137, 0.412, 1.235, 3.70, 11.1, 33.3, or 100 M (FIG. 5).

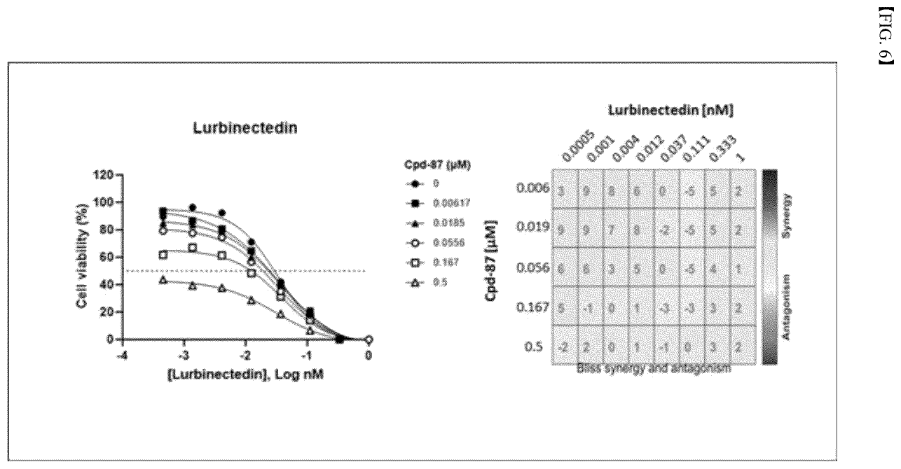

NCI-H1048 small cell lung cancer cell line was treated with compound-87 at concentrations of 0.006, 0.019, 0.056, 0.167, or 0.5 μM and rubinectedin, an alkylating agent, as a co-administered agent, at concentration of 0.0005, 0.001, 0.004, 0.012, 0.037, 0.111, 0.333, or 1 nM (FIG. 6).

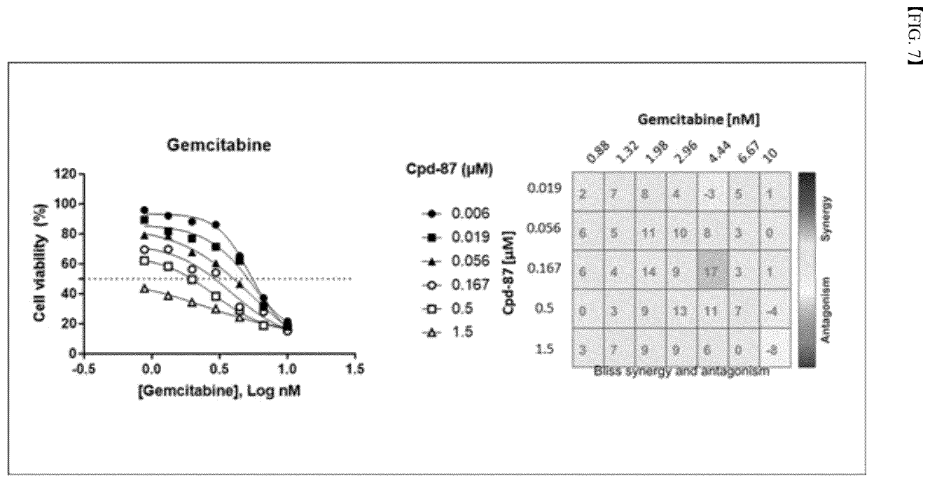

CAPAN-1 pancreatic cancer cell line was treated with compound-87 at concentrations of 0.019, 0.056, 0.167, 0.5, or 1.5 μM and gemcitabine, as a co-administered anti-metabolite, at concentration of 0.88, 1.32, 1.98, 2.96, 4.44, 6.67, or 10 nM (FIG. 7).

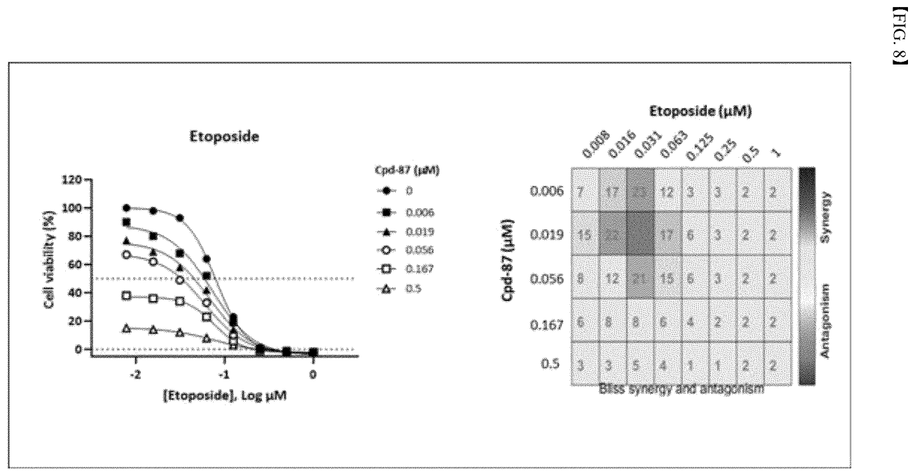

NCI-H1048 small cell lung cancer cell line was treated with compound-87 at concentrations of 0.006, 0.019, 0.056, 0.167, or 0.5 μM, and etoposide, a topoisomerase inhibitor as a co-administered agent, at concentrations of 0.008, 0.016, 0.031, 0.063, 0.125, 0.25, 0.5, or 1 μM (FIG. 8).

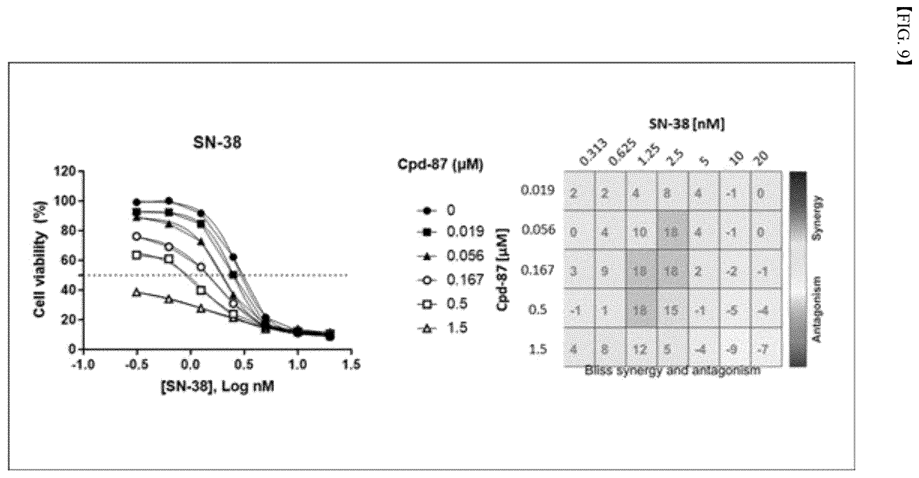

CAPAN-1 pancreatic cancer cell line was treated with compound-87 at concentrations of 0.019, 0.056, 0.167, 0.5, or 1.5 μM and SN-38, a topoisomerase inhibitor, as a co-administered agent, at concentration of 0.313, 0.625, 1.25, 2.5, 5, 10, or 20 nM (FIG. 9).

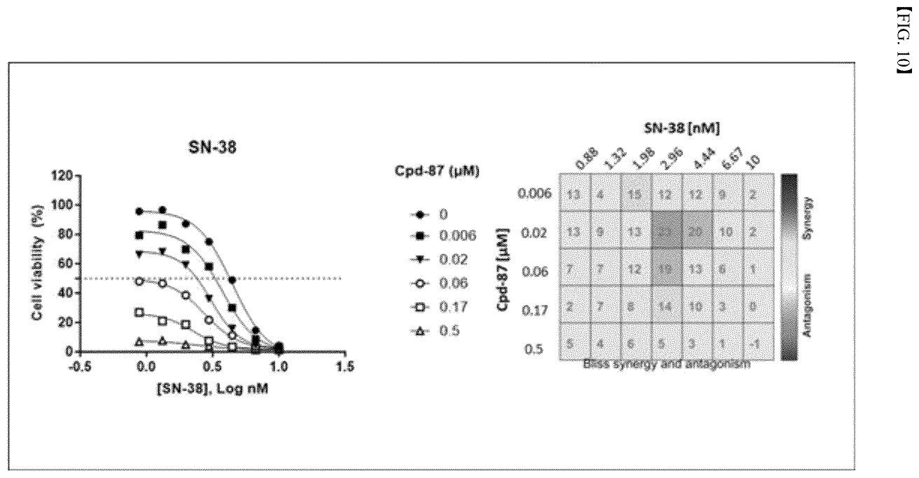

Mia PaCa-2 pancreatic cancer cell line was treated with compound-87 at concentrations of 0.006, 0.02, 0.06, 0.17, or 0.5 μM and SN-38, a topoisomerase inhibitor, as a co-administered agent, at concentration of 0.88, 1.32, 1.98, 2.96, 4.44, 6.67, or 10 nM (FIG. 10).

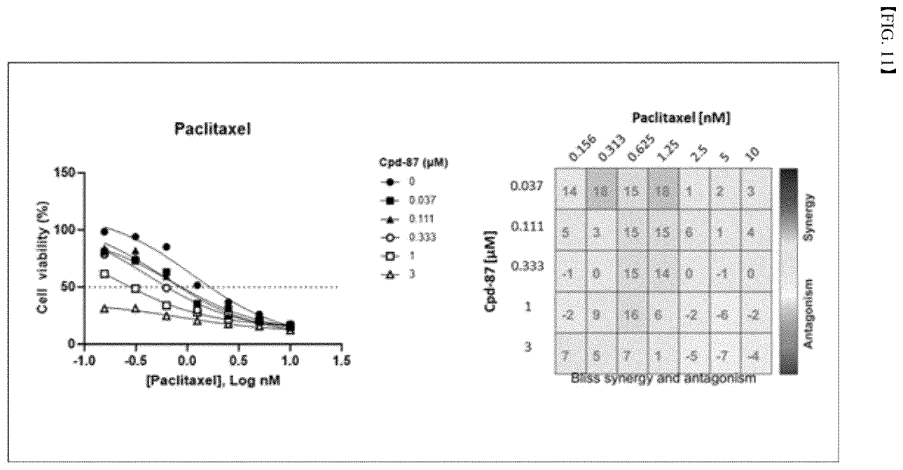

MDA-MB-231 breast cancer cell lines were treated with compound-87 at concentrations of 0.037, 0.111, 0.333, 1, or 3 μM and paclitaxel, a co-administered agent and microtubule inhibitor, at concentration of 0.156, 0.313, 0.625, 1.25, 2.5, 5, or 10 nM (FIG. 11).

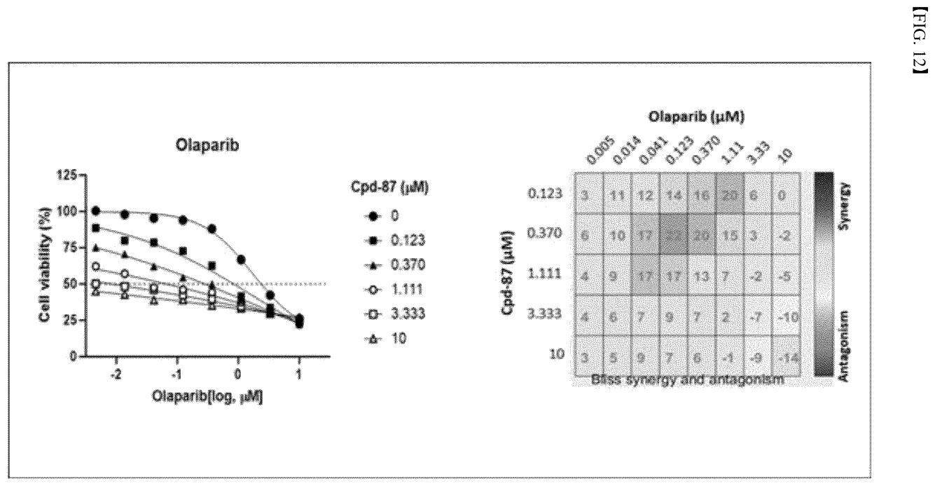

SNU-324 (BRCA2 mutant) pancreatic cancer cell line was treated with compound-87 at concentrations of 0.123, 0.370, 1.111, 3.333, or 10 μM, and olaparib, a PARP inhibitor, as a co-administered agent, at concentration of 0.0005, 0.014, 0.041, 0.123, 0.370, 1.11, 3.33, or 10 μM (FIG. 12).

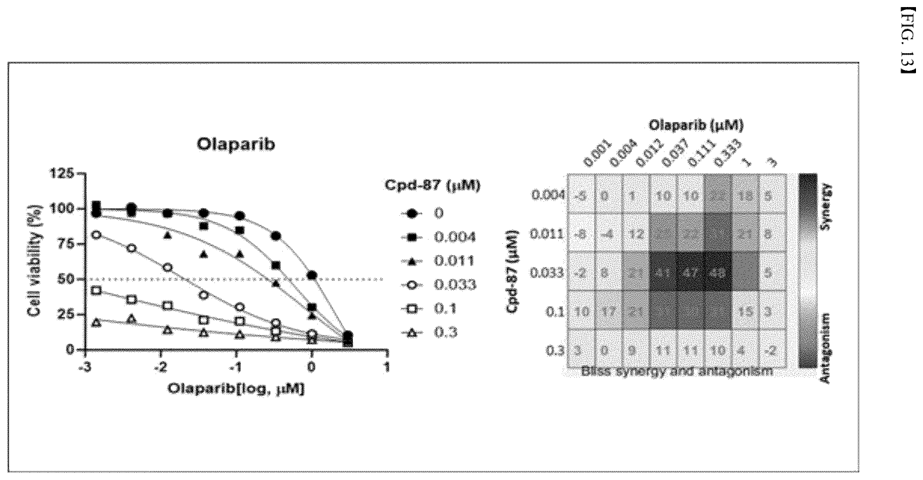

Mia PaCa-2 (wild-type BRCA2) pancreatic cancer cell line was treated with compound-87 at concentration of 0.004, 0.011, 0.033, 0.1, or 0.3 μM and the co-administered agent, the PARP inhibitor olaparib, at concentration of 0.0001, 0.004, 0.012, 0.037, 0.111, 0.333, 1, or μM (FIG. 13).

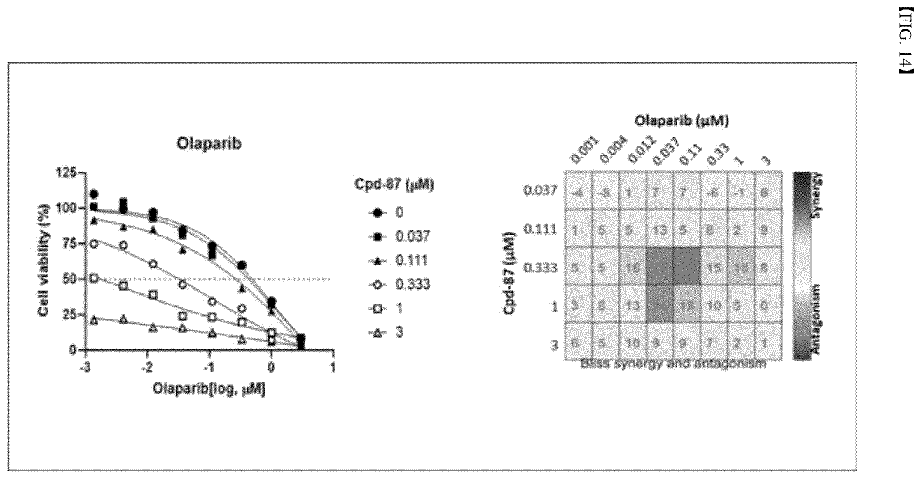

CAPAN-1 (BRCA2 mutant) pancreatic cancer cell lines were treated with compound-87 at concentrations of 0.037, 0.111, 0.333, 1, or 3 μM and olaparib, a PARP inhibitor, as a co-administered agent, at concentration of 0.001, 0.004, 0.012, 0.037, 0.11, 0.33, 1, or 3 μM (FIG. 14).

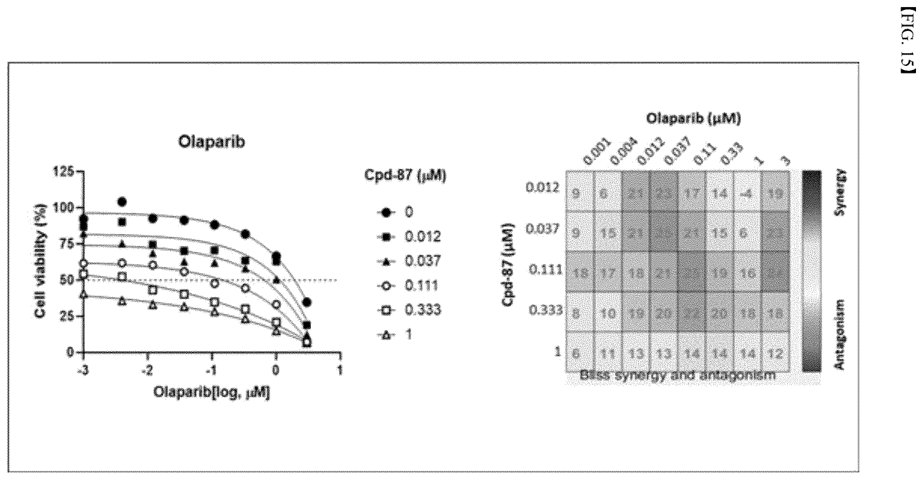

PANC-1 (wild-type BRCA1/BRCA2) pancreatic cancer cell line was treated with compound-87 at concentrations of 0.012, 0.037, 0.111, 0.333, or 1 μM and the co-administered agent, the PARP inhibitor olaparib, at concentration of 0.001, 0.004, 0.012, 0.037, 0.11, 0.33, 1, or 3 μM (FIG. 15).

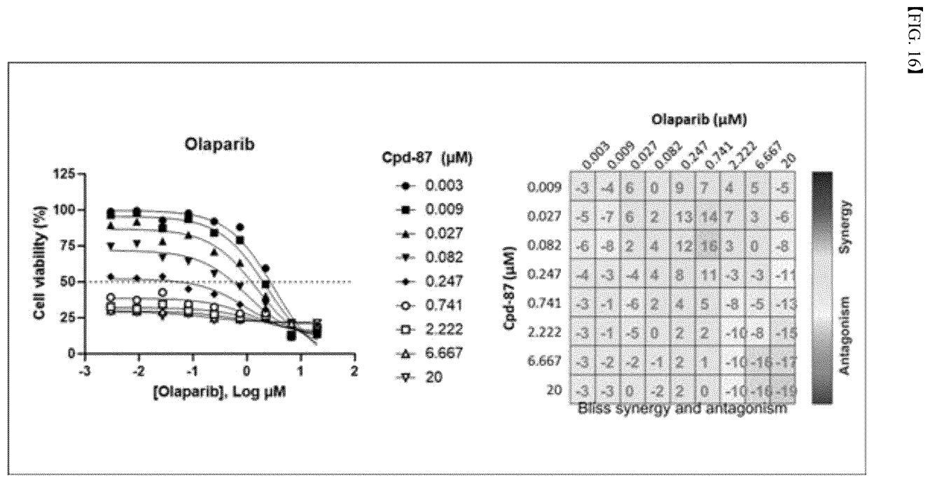

OVCAR3 (wild-type BRCA1/BRCA2) ovarian cancer cell line was treated with compound-87 at concentrations of 0.009, 0.027, 0.082, 0.247, 0.741, 2.222, 6.667, or 20 M, and olaparib, a PARP inhibitor as a co-administered agent, at concentrations of 0.003, 0.009, 0.027, 0.082, 0.247, 0.741, 2.222, 6.667, or 20 μM (FIG. 16).

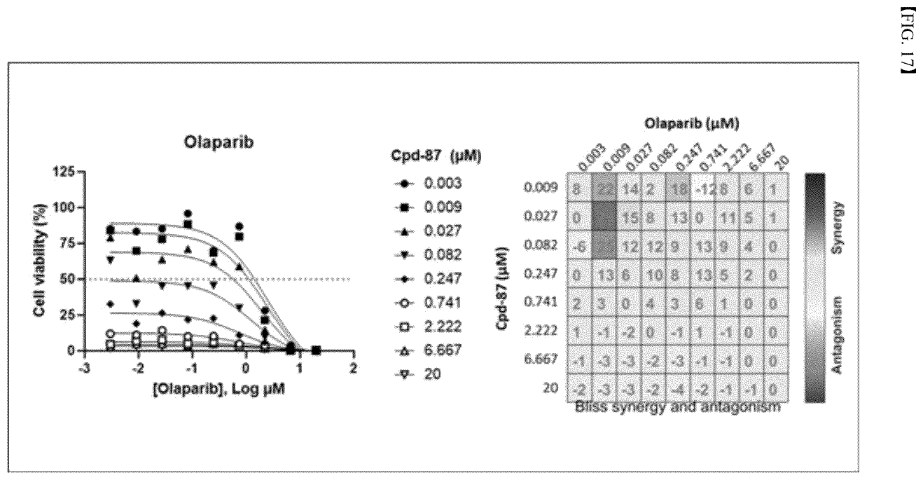

PA-1 (wild-type BRCA1/BRCA2) ovarian cancer cell line was treated with compound-87 at concentrations of 0.009, 0.027, 0.082, 0.247, 0.741, 2.222, 6.667, or 20 M, and olaparib, a PARP inhibitor as a co-administered agent, at concentration of 0.003, 0.009, 0.027, 0.082, 0.247, 0.741, 2.222, 6.667, or 20 μM (FIG. 17).

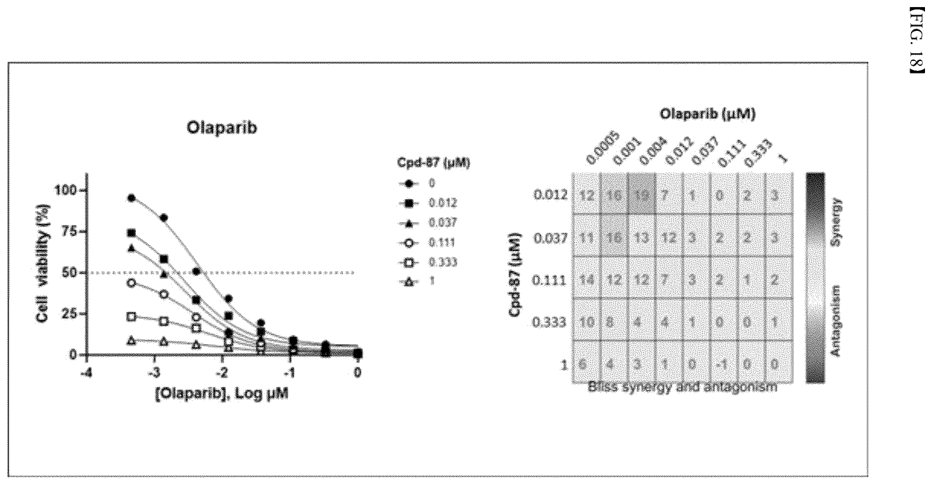

MDA-MB-231 (wild-type BRCA1/BRCA2) triple-negative breast cancer cell line was treated with compound-87 at concentration of 0.012, 0.037, 0.111, 0.333, or 1 μM and the co-administered agent, the PARP inhibitor olaparib, at concentration of 0.0005, 0.001, 0.004, 0.012, 0.037, 0.111, 0.333, or 1 μM (FIG. 18).

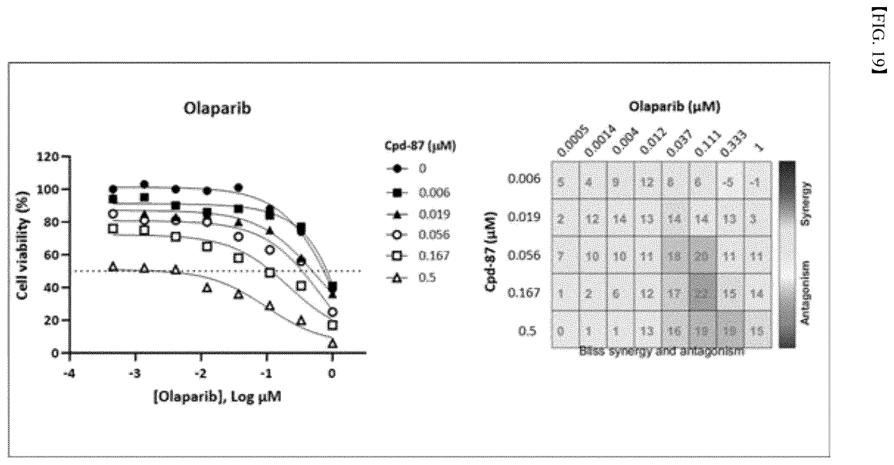

NCI-H1048 (wild-type BRCA1/BRCA2) small cell carcinoma cells were treated with compound-87 at concentrations of 0.006, 0.019, 0.056, 0.167, or 0.5 μM and the co-administered agent, the PARP inhibitor olaparib, at concentration of 0.0005, 0.0014, 0.004, 0.012, 0.037, 0.111, 0.333, or 1 μM (FIG. 19).

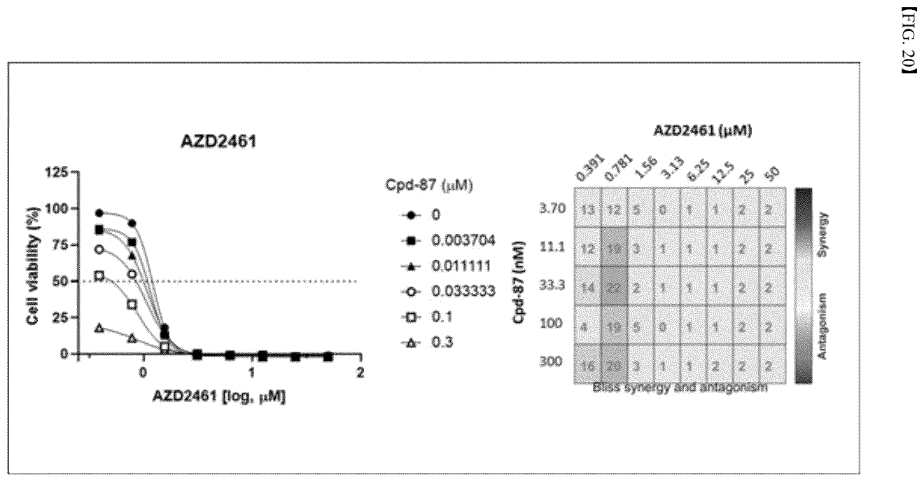

Mia PaCa-2 (wild-type BRCA2) pancreatic cancer cells were treated with compound-87 at concentrations of 300, 100, 33.3, 11.1, or 3.70 nM and the co-administered agent, the PARP inhibitor AZD2461, at concentration of 0.391, 0.781, 1.56, 3.13, 6.25, 12.5, 25, or 50 μM (FIG. 20).

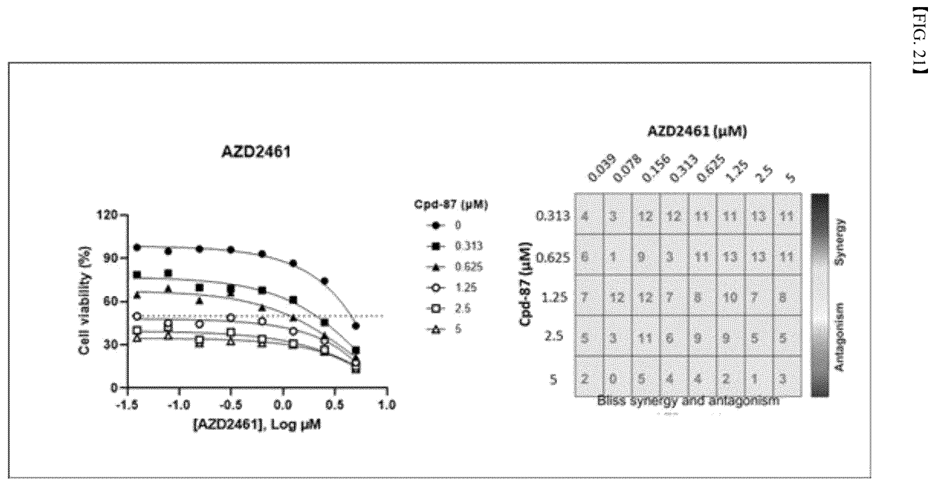

CAPAN-1 (BRCA2 mutant) pancreatic cancer cell lines were treated with compound-87 at concentrations of 0.313, 0.625, 1.25, 2.5, or 5 μM, and AZD2461, a PARP inhibitor as a co-administered agent, at concentration of 0.039, 0.078, 0.156, 0.313, 0.625, 1.25, 2.5, or 5 μM (FIG. 21).

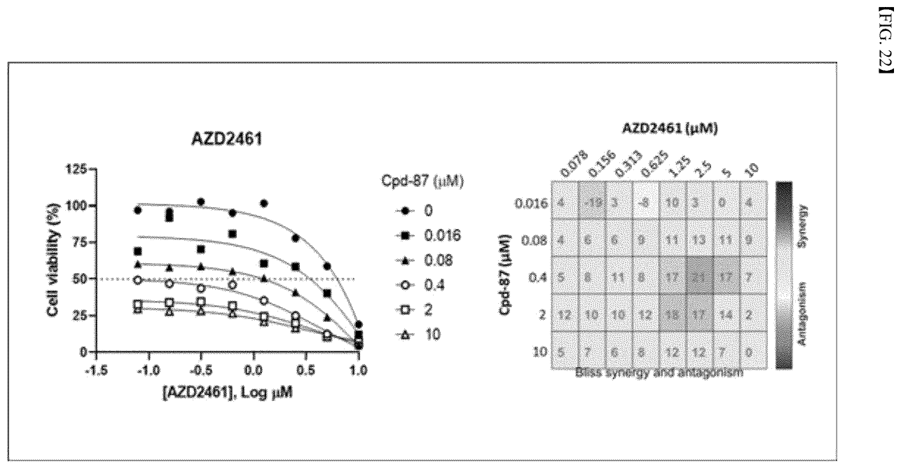

PANC-1 (wild-type BRCA1/BRCA2) pancreatic cancer cells were treated with compound-87 at concentrations of 0.016, 0.08, 0.4, 2, or 10 μM and AZD2461, a PARP inhibitor as a co-administered agent, at concentration of 0.078, 0.156, 0.313, 0.625, 1.25, 2.5, 5, or 10 μM (FIG. 22).

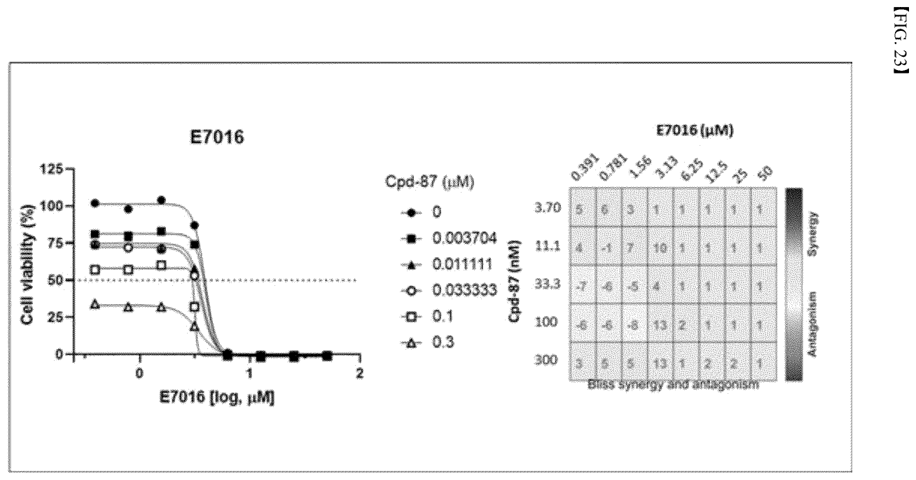

Mia PaCa-2 (wild-type BRCA2) pancreatic cancer cells were treated with compound-87 at concentrations of 300, 100, 33.3, 11.1, or 3.70 nM and the co-administered agent, the PARP inhibitor E7016, at concentration of 0.391, 0.781, 1.56, 3.13, 6.25, 12.5, 25, or 50 μM (FIG. 23).

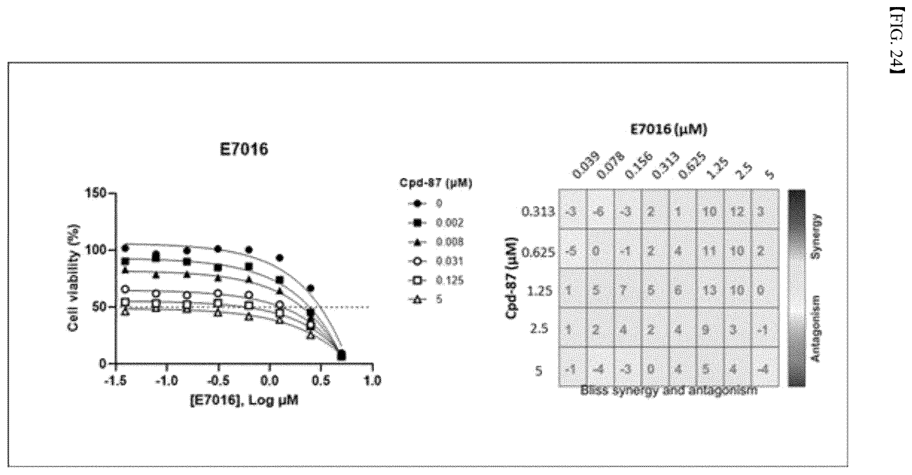

CAPAN-1 (BRCA2 mutant) pancreatic cancer cell lines were treated with compound-87 at concentrations of 0.313, 0.625, 1.25, 2.5, or 5 μM, and E7016, a PARP inhibitor as a co-administered agent, at concentration of 0.039, 0.078, 0.156, 0.313, 0.625, 1.25, 2.5, or 5 μM (FIG. 24).

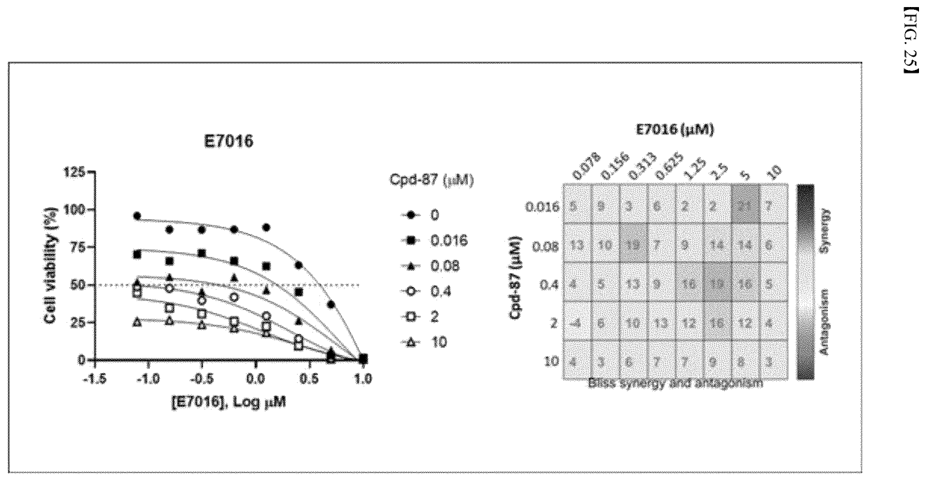

PANC-1 (wild-type BRCA1/BRCA2) pancreatic cancer cells were treated with compound-87 at concentrations of 0.016, 0.08, 0.4, 2, or 10 PM, and E7016, a PARP inhibitor as a co-administered agent at concentration of 0.078, 0.156, 0.313, 0.625, 1.25, 2.5, 5, or 10 μM (FIG. 25). Cell culture was terminated on days 7 to 9, and cell viability and synergistic effect were evaluated for each combination of concentrations.

Cell viability was measured by a colorimetric assay using water-soluble tetrazolium salt (WST). The optical density (OD) value is proportional to the number of actively proliferating cells. The untreated group means only the culture medium and WST without cells, and the DMSO-treated group means the group treated with WST alone without any drug.

### 2-3: Data Analysis

Results are normalized to DMSO-treated groups as 100% and expressed as the percent of viable cells upon compound addition. Data were modeled using a nonlinear regression curve fit with a four-parameter sigmoidal dose-response curve for cell viability versus concentration using GraphPad Prism 9 (GraphPad Software). The 50% growth inhibition value (IC₅₀) corresponds to the concentration that crosses the midpoint of the survival rate.

Cell viability %=[(mean OD of group treated with test substance−mean OD of untreated group)/(mean OD of group treated with DMSO−mean OD of untreated group)]%

The synergy of each compound was analyzed using Combenefit software (Cambridge Research UK, Cambridge Institute, University of Cambridge, United Kingdom), and synergy was evaluated by using Blliss (Ref: Di Veroli et al., 2016, Bioinformatics 32(18):2866-2868). The software calculates a Blliss score for each drug combination, where a Blliss score value of −10 or less indicates antagonism, and a Blliss score value of 10 or greater indicates strong synergy (Reference: SynergyFinder_user documentation).

As illustrated in FIGS. 1 to 25 and further discussed in the “In Vivo Data” section below, combinations of Compound-87, Compound-112, and Compound-271 with various DNA damage inducers demonstrated enhanced inhibition effects of cell viability and drug-drug synergy (Blliss score>10) for the combination treatment, compared to single agent. In FIGS. 1 to 25, Cpd-87, 112, and 271 represent Compound-87, Compound-112, and Compound-271, respectively, and the Blliss score is an indicator of drug-drug synergy.

### Example 3: Evaluation of Combination Efficacy in an Animal Model Using NCI-H1048 Cells

For in vivo efficacy study in a subcutaneous xenograft mouse model of human small cell lung cancer cells NCI-H1048, co-administration of compound-87 as a PRMT5 inhibitor according to an example of the present invention and cisplatin as an alkylating agent of a chemotherapeutic agent, was performed. To produce a small cell lung cancer xenograft model, 10 million of NCI-H1048 cells were injected subcutaneously into the right flank of female nude mice, and tumor growth was monitored.

To evaluate the anticancer effect, when the tumor volume reached approximately 162 mm³ on average, the mice were divided into four groups. The all group included a solvent-treated group (Vehicle 1 (Compound-87)+Vehicle 2 (cisplatin) group), a Compound-87 monotherapy group (50 mg/kg), a cisplatin monotherapy group (4 mg/kg), and combination therapy group of Compound-87 and cisplatin. The Compound-87 treatment group was administered orally on a 3-day administration/4-day non-administration schedule, and cisplatin was administered intraperitoneally once a week for 22 days (Day 1-Day 23). All mice were euthanized on Day 22 (Day 23).

In the group administered compound-87 and cisplatin alone, a tumor growth inhibition effect was observed, and a greater tumor growth inhibition effect was observed in the combination therapy group administered compound-87 and cisplatin, compared to the group administered compound-87 and cisplatin alone. On day 23, tumor growth inhibition rates of 62.0% and 62.9% were observed in the groups administered compound-87 and 4 mg/kg cisplatin alone, respectively. When compound-87 and cisplatin were combined, a partial response was observed in all mice, and a partial response of 60.6% was observed, showing a greater tumor growth inhibition effect compared to the group administered compound-87 and cisplatin alone.

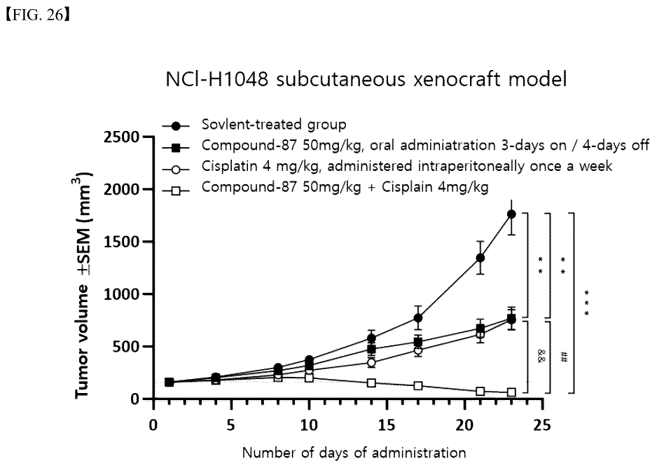

Furthermore, as shown in FIG. 26, when co-administration of compound-87 and cisplatin were performed in a subcutaneous xenograft mouse model of human small cell lung cancer cell NCI-H1048, they showed a higher anticancer effect than single administration of each drug. FIG. 26 is the result of Dunnett's T3′ multiple comparison test following one-way ANOVA using the Brown-Forsythe/Welch test (**p<0.01, ***p<0.001, ****p<0.0001 vs solvent treatment group; ^{##}p<0.01, ^{###}p<0.001 vs treatment group of compound 50 mg/kg; & p<0.05, && p<0.01 vs treatment group of cisplatin 4 mg/kg).

### Example 4: Evaluation of Combination Efficacy in an Animal Model Using CAPAN-1 Cells

For in vivo efficacy studies in a subcutaneous xenograft mouse model of human pancreatic cancer cells CAPAN-1, co-administration of compound-87 as a PRMT5 inhibitor according to one example of the present invention and Gemcitabine as an anti-metabolite of a chemotherapeutic agent, was performed.

To produce a pancreatic cancer xenograft model, 5 million CAPAN-1 cells were injected subcutaneously into the right flank of female nude mice, and tumor growth was monitored.

To evaluate the anticancer effect, when the tumor volume reached approximately 185 mm³ on average, the mice were divided into six groups. The all group included the solvent-treated group (vehicle group), the compound-87 monotherapy group (25 mg/kg or 50 mg/kg), the gemcitabine monotherapy group (50 mg/kg), combination therapy group of the compound-87 (25 mg/kg or 50 mg/kg), and the gemcitabine. The vehicle A group or the compound-87 treatment group was administered orally on a 3-day administration/4-day non-administration schedule, and the vehicle B group or the gemcitabine treatment group was administered intraperitoneally twice a week.

On Day 31 of administration, the tumor volume of the solvent-treated group reached 1,017 mm³. When administered in combination with gemcitabine, the treatment groups of Compound-87 25 mg/kg and 50 mg/kg significantly suppressed tumor growth on Day 31, with tumor volumes of 379 and 235 mm³, respectively, showing higher tumor growth inhibition efficacy than the Compound-87 monotherapy group and the gemcitabine monotherapy group at the same dose.

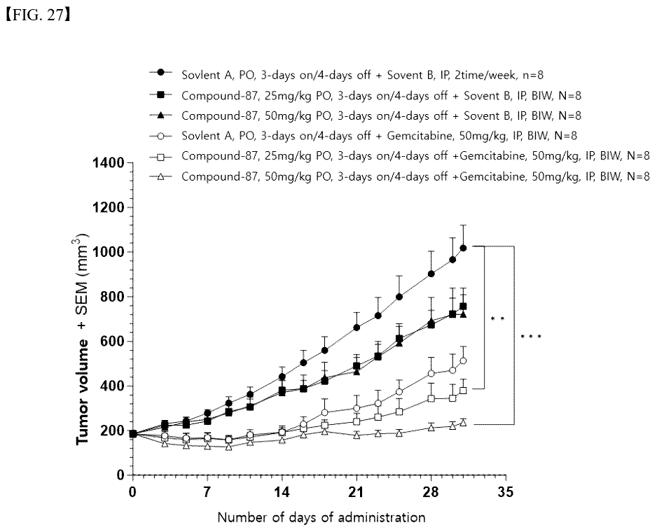

Furthermore, as shown in FIG. 27, when compound-87 and gemcitabine were administered together, a higher anticancer effect in a subcutaneous xenograft mouse model of human pancreatic cancer cell CAPAN-1, was observed compared to single administration of each drug. FIG. 27 shows the results of the Kruskal-Waliis test followed by Dunn's multiple comparison test (** p<0.01, *** p<0.001).

### Example 5: Evaluation of Combination Efficacy in an Animal Model Using Mia PaCa-2 Cells

### 5-1: Experimental Method

For in vivo efficacy study in a subcutaneous xenograft mouse model of human pancreatic cancer cells Mia PaCa-2, co-administration of compound-87 as a PRMT5 inhibitor according to an example of the present invention and Gemcitabine as an anti-metabolite of a chemotherapeutic agent, was performed. To produce a pancreatic cancer xenograft model, 5 million Mia PaCa-2 cells were injected subcutaneously into the right flank of female nude mice, and tumor growth was monitored.

To evaluate the anticancer effect, mice were divided into six groups when the tumor volume reached approximately 150 mm³ on average. All group included a solvent-treated group (vehicle group), a compound-87 monotherapy group (12.5 mg/kg or 50 mg/kg), a gemcitabine monotherapy group (60 mg/kg), and combination therapy group of compound-87 and gemcitabine. The solvent-treated group (vehicle group) or the compound-87 treatment group was administered orally on a 3-day administration/4-day non-administration schedule, and gemcitabine was administered intraperitoneally twice a week for 24 days (Day 0-Day 23). All mice were euthanized on Day 24, 24 hours after the last administration.

In the compound-87 monotherapy group, the tumor growth inhibition effect was observed in a compound-87 concentration-dependent manner, and in the combination therapy group of compound-87 and gemcitabine, a greater tumor growth inhibition effect was observed compared to the compound-87 monotherapy group. On day 24, tumor growth inhibition rates of 42.6% and 56.0% were observed in the compound-87 25 mg/kg monotherapy group and the 50 mg/kg monotherapy group, respectively. When combined with gemcitabine 60 mg/kg, the compound-87 25 mg/kg monotherapy group and the 50 mg/kg monotherapy group showed tumor growth inhibition rates of 67.7% and 77.0%, respectively, indicating compound-87 concentration-dependent manner and greater tumor growth inhibition effect compared to the monotherapy group.

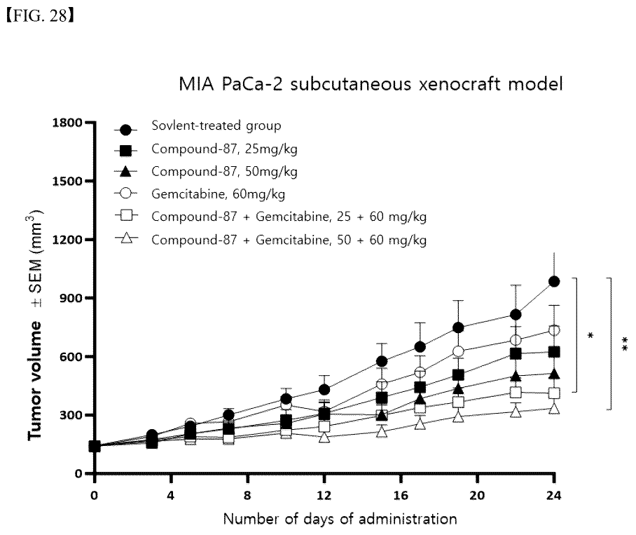

Furthermore, as shown in FIG. 28, when compound-87 and gemcitabine were administered together in a subcutaneous xenograft mouse model of human pancreatic cancer cells Mia PaCa-2, a higher anticancer effect was observed compared to single administration of each drug. FIG. 28 shows the results of the Kruskal-Waliis test followed by Dunn's multiple comparison test (* p<0.05, ** p<0.01).

### 5-2: Test for In Vivo Target Inhibition Ability in Animal

Quantitative reduction levels of symmetric dimethyl arginine (SDMA) in tumor samples harvested from a subcutaneous xenograft mouse model of Mia PaCa-2 were measured by enzyme-linked immunosorbent assay (ELISA).

Reagents: Bradford solution (catalog no. 500-0006) was purchased from Bio-rad (USA). SDMA antibody (catalog no. 13222s) was purchased from Cell signaling Technology (USA). SmD3 antibody (catalog no. ap12451a) was purchased from Abgent (USA). Secondary antibody (catalog no. ab6721) and TMB substrate (catalog no. ab210902) were purchased from Abcam (UK).

Experimental procedure: Tumor tissue transplanted into mice was extracted, cells were lysed, and quantified with Bradford solution. 5-10 g of protein per sample was diluted with carbonate-bicarbonate buffer, dispensed into a 96-well plate, and reacted at room temperature for 2 hours. After washing three times with phosphate-buffered saline (PBST) containing 0.05% Tween-20, 200 μL of PBST containing 5% bovine serum albumin (BSA) (BSA-PBST) was added, and reacted at room temperature for 2 hours. After washing three times with PBST, 100 μL of SDMA antibody and SmD3 antibody were diluted in BSA-PBST, each was dispensed, and reacted overnight at 4° C. After washing three times with PBST on next day, 100 μL of secondary antibody diluted in BSA-PBST was added, and reacted at room temperature for 1 hour. After washing three times with PBST, 100 μL of TMB substrate was added and reacted at room temperature for 10-20 minutes, and 100 μL of 1N sulfuric acid solution was added to terminate the reaction. Then, the degree of SDMA inhibition by the compound was calculated by measuring the absorbance at 450 nm.

Compound-87 administration for 3 days/no administration for 4 days for 24 days significantly reduced the amount of SDMA in the tumor in a Compound-87 dose-dependent manner, regardless of whether it was co-administered with gemcitabine. The SDMA amounts in the tumor samples were 37.4% (62.6% inhibition) and 27.7% (72.3% inhibition) in treatment groups with the 25 mg/kg and 50 mg/kg of Compound-87, respectively, compared to the vehicle group. Co-administration with 60 mg/kg of gemcitabine reduced the SDMA amounts to 33.5% (66.5% inhibition) and 24.9% (75.1% inhibition) in the treatment groups of 25 mg/kg and 50 mg/kg of Compound-78, respectively.

Therefore, the combination of a PRMT5 inhibitor according to one example of the present invention and gemcitabine demonstrated enhanced inhibition of tumor SDMA compared to single agent.

### Example 6: Evaluation of Combination Efficacy in an Animal Model Using Patient-Derived CTG-1932 Cells

For in vivo efficacy studies in a subcutaneous xenograft mouse model of CTG-1932, a human non-small cell lung cancer patient-derived cell line, combination therapy of compound-87 as a PRMT5 inhibitor according to one example of the present invention and with paclitaxel as a chemotherapy agent, was performed at Champions Oncology (USA).

To produce a non-small cell lung cancer xenograft model, CTG-1932 tumor stock was grown and harvested from primary mice and then transplanted subcutaneously into the left flank of immunocompromised female mice as a 5×5×5 mm³ tumor fragment. When the tumor volume reached approximately 150-300 mm³, the mice were separated into four groups of 10 each. All group consisted of a solvent-treated group (vehicle group), a compound-87 monotherapy group (50 mg/kg, oral administration, 3 days on/4 days off), a paclitaxel monotherapy group (20 mg/kg, intraperitoneal administration once a week), and combination treatment group of compound-87 (50 mg/kg, oral administration, 3 days on/4 days off) and paclitaxel (20 mg/kg, intraperitoneal administration once a week). tumor volume and body weight were measured twice a week.

Oral treatment with 50 mg/kg of compound-87 as a single agent showed 39% of tumor growth inhibition effect compared to the solvent-treated group on day 9, which was not significant, while single treatment with 20 mg/kg of paclitaxel showed a significant tumor growth inhibition effect of 75% compared to the solvent-treated group. In addition, the group administered compound-87 and paclitaxel together showed 89% of tumor growth inhibition compared to the solvent-treated group.

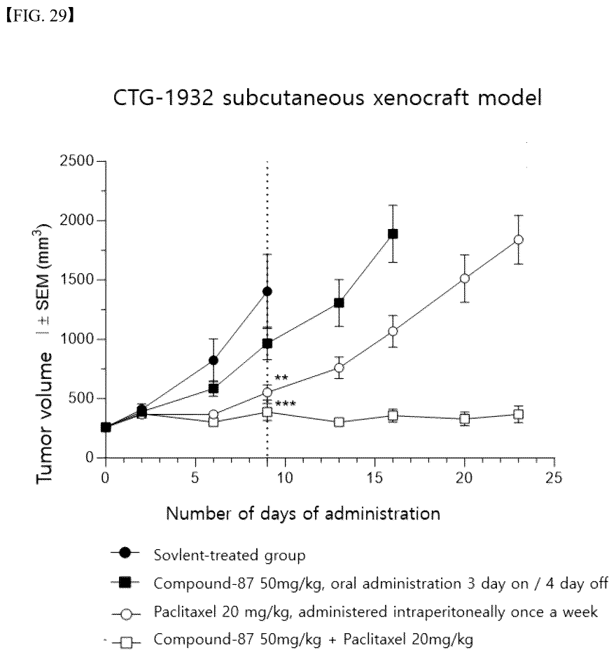

Furthermore, FIG. 29 shows a significant tumor growth inhibition effect when compound-87 was combined with paclitaxel as a chemotherapy agent in a CTG-1932 xenograft mouse model. FIG. 29 is the result of Dunnett's multiple comparison test following one-way ANOVA (** p<0.01, *** p<0.001).

### Example 7: Gene Expression Analysis in an Animal Model Using CTG-2534 Cells

In order to analyze changes in gene expression by PRMT5 inhibitor treatment according to one example of the present invention, patient-derived cells CTG-2534 were xenografted into mice subcutaneously, and then the mice were divided into groups, when the tumor volume reached 80 to 300 mm³. The solvent-treated group (vehicle group) or the compound-87 treatment group was orally administered on a 3-day on/4-day off schedule. When the tumor volume of the solvent-treated group reached 955 mm³, the tumors were separated and performed for gene expression analysis.

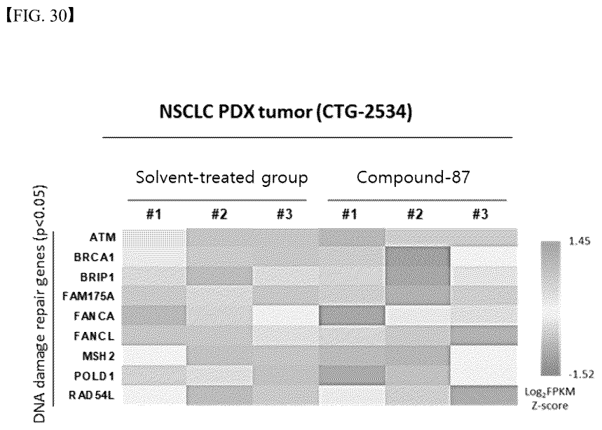

FIG. 30 is a heat map showing the change in expression of DNA damage repair genes using Z-score values for Log₂FPKM, after compound-87 treatment were performed in patient-derived lung cancer GTG-2393 cell xenograft mice. In FIG. 30, the solvent-treated group is shown in red (positive value), and the compound-87-treated group is shown in blue (negative value).

The results show that when compound-87 as a PRMT5 inhibitor according to an example of the present invention is treated, the reduced expression of DNA damage repair genes does make actively dividing cancer cells not to respond to DNA damage, which can cause problems in cancer cell differentiation and survival due to increased replication stress and accumulation of damaged DNA.

Therefore, the PRMT5 inhibitor according to one example of the present invention acts on cancer cells lacking homologous recombination (BRCA1/BRACA2 mutation) which is required for DSB (double strand break) repair, making it difficult for compensation of DNA repair to occur, causing DNA damage and inducing cancer cell death, when combined with a PARP inhibitor to cause synthetic lethality. In particular, this action can serve as a basis for showing a synergistic effect on BRCA wild-type cancer cells in addition to BRCA1/2 mutant cancer cells.

1. A pharmaceutical combination, comprising a combination of
a PRMT5 inhibitor of following Chemical Formula 1, optical isomer, stereoisomer or isotope variant thereof, or a pharmaceutically acceptable salt thereof; and
a DNA damage inducing substance: 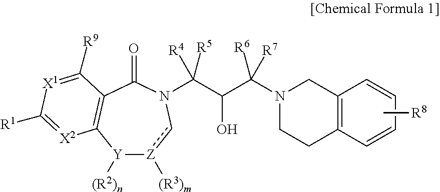
wherein,
X¹ and X² are each independently carbon or nitrogen;
Y is carbon, oxygen or nitrogen;
Z is carbon;
n is an integer of 0 or 1;
m is an integer from 0 to 2; [symbol]
is a single bond or double bond;
R¹ is -D-R¹⁰; wherein D is a direct bond, —O—, —C(═O)—, —C≡C— or —CR¹¹R¹²—; R¹⁰ is hydrogen, halo, hydroxy, cyano, alkyl, hydroxyalkyl, haloalkyl, haloalkylsulfonate, dialkylamino, alkylaminoalkyl, dialkylaminoalkyl, dialkylaminocarbonylalkyl, saturated or unsaturated carbocyclyl, saturated or unsaturated heterocyclyl, saturated or unsaturated carbocyclyl-alkyl, or saturated or unsaturated heterocyclyl-alkyl; R¹¹ and R¹² are each independently hydrogen, hydroxy or alkyl; the carbocycle or heterocycle may be substituted with one or more substituents selected from the group consisting of hydroxy, halo, oxo, formyl (—CHO), nitrile, alkyl, alkoxy, hydroxyalkyl, hydroxyhaloalkyl, alkoxyalkyl, haloalkyl, nitrilealkyl, alkylcarbonyl, alkylthiocarbonyl, alkoxycarbonyl, haloalkylcarbonyl, carbocyclyl, carbocyclylcarbonyl, (alkyl)(haloalkyl)amino, (alkyl)(heterocyclyl)amino, heterocyclyl and heterocyclyl-alkyl;
R², R³, R⁴, R⁵, R⁶ and R⁷ are each independently hydrogen or alkyl;
R⁸ is hydrogen, halo, alkyl, alkoxy or amino; and
R⁹ is hydrogen, halo, or alkyl.

2. The pharmaceutical combination of claim 1, wherein
X¹ and X² are each independently CH or N;
R² is hydrogen or C₁-C₇ alkyl;
R³ is hydrogen or C₁-C₇ alkyl;
R⁴, R⁵, R⁶ and R⁷ are each independently hydrogen or C₁-C₇ alkyl;
R⁸ is hydrogen, halo, C₁-C₇ alkyl, C₁-C₇ alkoxy or amino;
R⁹ is hydrogen, halo or C₁-C₇ alkyl; [symbol]
is a single bond;
when n is 0, Y is CH₂, 0, or NH; when n is 1, Y is CH or N; and
when m is 0, Z is CH₂ or CH; when m is 1, Z is CH or C; and when m is 2, Z is C.

3. The pharmaceutical combination of claim 1, wherein
R¹ is -D-R¹⁰;
D is a direct bond, —O—, —C(═O)—, —C≡C—, or —CR¹¹R¹²—;
R¹⁰ is hydrogen, halo, hydroxy, cyano, C₁-C₇ alkyl, hydroxy-C₁-C₇ alkyl, halo-C₁-C₇ alkyl, halo-C₁-C₇ alkylsulfonate, di(C₁-C₇ alkyl)amino, C₁-C₇ alkylamino-C₁-C₇ alkyl, di(C₁-C₇ alkyl)amino-C₁-C₇ alkyl, di(C₁-C₇ alkyl)aminocarbonyl-C₁-C₇ alkyl, saturated or unsaturated C₃-C₁₀ carbocyclyl, saturated or unsaturated 4- to 10-membered heterocyclyl, saturated or unsaturated C₃-C₁₀ carbocyclyl-C₁-C₇ alkyl, or saturated or unsaturated 4- to 10-membered heterocyclyl-alkyl; and
R¹¹ and R¹² are each independently hydrogen, hydroxy or C₁-C₇ alkyl.

4. (canceled)

5. The pharmaceutical combination of claim 1,
wherein R¹⁰ is halo, C₁-C₇ alkyl, hydroxy-C₁-C₇ alkyl, halo-C₁-C₇ alkyl, saturated or unsaturated C₃-C₁₀ carbocyclyl, saturated or unsaturated 4- to 10-membered heterocyclyl, or saturated or unsaturated 4- to 10-membered heterocyclyl-alkyl; or
wherein the carbocycle or the heterocycle is optionally substituted with one to five substituents selected from the group consisting of hydroxy, halo, oxo, formyl, nitrile, C₁-C₇ alkyl, C₁-C₇ alkoxy, hydroxy-C₁-C₇ alkyl, hydroxyhalo-C₁-C₇ alkyl, C₁-C₇ alkoxy-C₁-C₇ alkyl, halo-C₁-C₇ alkyl, nitrile-C₁-C₇ alkyl, C₁-C₇ alkylcarbonyl, C₁-C₇ alkylthiocarbonyl, C₁-C₇ alkoxycarbonyl, halo-C₁-C₇ alkylcarbonyl, saturated or unsaturated C₃-C₁₀ carbocyclyl, saturated or unsaturated C₃-C₁₀ carbocyclylcarbonyl, (C₁-C₇ alkyl)(halo-C₁-C₇ alkyl)amino, (C₁-C₇ alkyl)(saturated or unsaturated 4- to 10-membered heterocyclyl)amino, saturated or unsaturated 4- to 10-membered heterocyclyl, and saturated or unsaturated 4- to 10-membered heterocyclyl-C₁-C₇ alkyl.

6. The pharmaceutical combination of claim 5, wherein the carbocycle or the heterocycle is optionally substituted with one to five substituents selected from the group consisting of hydroxy, halo, formyl, C₁-C₇ alkyl, hydroxy-C₁-C₇ alkyl, C₁-C₇ alkoxy-C₁-C₇ alkyl, halo-C₁-C₇ alkyl, C₁-C₇ alkylcarbonyl, C₁-C₇ alkoxycarbonyl, halo-C₁-C₇ alkylcarbonyl, saturated or unsaturated C₃-C₁₀ carbocyclyl, saturated or unsaturated C₃-C₁₀ carbocyclylcarbonyl, (C₁-C₇ alkyl)(halo-C₁-C₇ alkyl)amino, (C₁-C₇ alkyl)(saturated or unsaturated 4- to 10-membered heterocyclyl)amino, and saturated or unsaturated 4- to 10-membered heterocyclyl-C₁-C₇ alkyl.

7. The pharmaceutical combination of claim 1, wherein the heterocycle is optionally substituted with one or two substituents selected from the group consisting of hydroxy, halo, formyl, C₁-C₇ alkyl, hydroxy-C₁-C₇ alkyl, C₁-C₇ alkoxy-C₁-C₇ alkyl, halo-C₁-C₇ alkyl, C₁-C₇ alkylcarbonyl, C₁-C₇ alkoxycarbonyl, halo-C₁-C₇ alkylcarbonyl, saturated or unsaturated C₃-C₁₀ carbocyclyl, saturated or unsaturated C₃-C₁₀ carbocyclylcarbonyl, and saturated or unsaturated 4- to 10-membered heterocyclyl-C₁-C₇ alkyl; or
wherein the carbocyclyl is optionally substituted with one or two substituents selected from the group consisting of halo-C₁-C₇ alkyl, (C₁-C₇ alkyl)(halo-C₁-C₇ alkyl) amino, and (C₁-C₇ alkyl)(saturated or unsaturated 4- to 10-membered heterocyclyl)amino.

8. (canceled)

9. The pharmaceutical combination of claim 1, wherein
R¹ is -DR¹⁰, D is a direct bond;
R¹⁰ is hydrogen, halo, cyano, C₁-C₇ alkyl, halo-C₁-C₇ alkyl, di(C₁-C₇ alkyl)amino-C₁-C₇ alkyl, saturated or unsaturated 4- to 10-membered heterocyclyl, saturated or unsaturated C₃-C₁₀ carbocyclyl, or saturated or unsaturated 4- to 10-membered heterocyclyl-alkyl; and
the heterocyclyl is optionally substituted with one or two substituents selected from the group consisting of hydroxy, halo, formyl, C₁-C₇ alkyl, C₁-C₇ alkoxy, hydroxy-C₁-C₇ alkyl, halo-C₁-C₇ alkyl, nitrile-C₁-C₇ alkyl, C₁-C₇ alkylcarbonyl, C₁-C₇ alkoxy-C₁-C₇ alkyl, C₁-C₇ alkoxycarbonyl, halo-C₁-C₇ alkylcarbonyl, and saturated or unsaturated 4- to 10-membered heterocyclyl.

10. (canceled)

11. (canceled)

12. The pharmaceutical combination of claim 1, wherein the heterocycle is a saturated or unsaturated 4- to 8-membered hydrocarbon containing one or two heteroatoms selected from nitrogen and oxygen; or
wherein the carbocyclyl is selected from the group consisting of cyclohexane, cyclohexene, cyclopropane, cyclobutane, and cyclopentane.

13. The pharmaceutical combination of claim 1, wherein the heterocycle is selected from the group consisting of tetrahydropyridine, dihydropyridine, piperidine, dihydropyran, tetrahydropyran, pyrrolidine, 2-oxa-6-azaspiroheptane, azetidine, morpholine, 3,3a,4,5,6,6a-hexahydro-1H-cyclopenta[c]pyrrole, oxazepane, 2-oxa-5-azabicyclo[2.2.1]heptane, pyridyl, tetrahydrofuran, 8-azabicyclo[3.2.1]octane, piperazine, 2-azaspiro[3.3]heptane, 2-oxa-7-azaspiro[3.4]octane, 2-azabicyclo[2.2.1]heptane, 3-oxa-8-azabicyclo[3.2.1]octane, 3,4-dihydro-1H-pyrrolo[1,2-a]pyrazine, 3,4,6,7,8,8a-hexahydro-1H-pyrrolo[1,2]-a]pyrazine, pyrimidine, pyrazole, 2-oxa-7-azaspiro[3.5]nonane, and oxetane.

14. The pharmaceutical combination of claim 13, wherein the heterocycle is selected from the group consisting of tetrahydropyridine, dihydropyridine, piperidine, tetrahydropyran, pyrrolidine, 2-oxa-6-azaspiroheptane, azetidine, morpholine, 3,3a,4,5,6,6a-hexahydro-1H-cyclopenta[c]pyrrole, pyridyl, 8-azabicyclo[3.2.1]octane, piperazine, 2-azaspiro[3.3]heptane, pyrazole, and oxetane.

15. (canceled)

16. The pharmaceutical combination of claim 1, wherein
D is a direct bond, —O—, —C(═O)— or —C≡C—;
R¹⁰ is halo, C₁-C₇ alkyl, hydroxy-C₁-C₇ alkyl, halo-C₁-C₇ alkyl, saturated or unsaturated C₃-C₁₀ carbocyclyl, saturated or unsaturated 4- to 10-membered heterocyclyl, or saturated or unsaturated 4- to 10-membered heterocyclyl-alkyl;
the carbocycle or heterocycle is optionally substituted with one to five substituents selected from the group consisting of hydroxy, halo, formyl, C₁-C₇ alkyl, hydroxy-C₁-C₇ alkyl, C₁-C₇ alkoxy-C₁-C₇ alkyl, halo-C₁-C₇ alkyl, C₁-C₇ alkylcarbonyl, C₁-C₇ alkoxycarbonyl, halo-C₁-C₇ alkylcarbonyl, saturated or unsaturated C₃-C₁₀ carbocyclyl, saturated or unsaturated C₃-C₁₀ carbocyclylcarbonyl, (C₁-C₇ alkyl)(halo-C₁-C₇ alkyl)amino, (C₁-C₇ alkyl)(saturated or unsaturated 4- to 10-membered heterocyclyl)amino, and saturated or unsaturated 4- to 10-membered heterocyclyl-C₁-C₇ alkyl;
the heterocyclyl is selected from the group consisting of tetrahydropyridine, dihydropyridine, piperidine, tetrahydropyran, pyrrolidine, 2-oxa-6-azaspiroheptane, azetidine, morpholine, 3,3a,4,5,6,6a-hexahydro-1H-cyclopenta[c]pyrrole, pyridyl, 8-azabicyclo[3.2.1]octane, piperazine, 2-azaspiro[3.3]heptane, pyrazole, and oxetane; and
the carbocyclyl is selected from the group consisting of cyclohexane, cyclohexene, and cyclopropane.

17. The pharmaceutical combination of claim 1, wherein the PRMT5 inhibitor of Chemical Formula 1 is selected from the group consisting of (1) to (277):
(1) 4-[3-(3,4-dihydro-1H-isoquinolin-2-yl)-2-hydroxy-propyl]-8-methoxy-2,3-dihydro-1,4-benzoxazepin-5-one;
(2) 2-[3-(3,4-dihydro-1H-isoquinolin-2-yl)-2-hydroxy-propyl]-7-methoxy-4,5-dihydro-3H-2-benzazepin-1-one;
(3) 2-[3-(3,4-dihydro-1H-isoquinolin-2-yl)-2-hydroxy-propyl]-7-hydroxy-4,5-dihydro-3H-2-benzazepin-1-one;
(4) 2-[3-(3,4-dihydro-1H-isoquinolin-2-yl)-2-hydroxy-propyl]-7-(trifluoromethoxy)-4,5-dihydro-3H-2-benzazepin-1-one;
(5) 4-[(2R)-3-(3,4-dihydro-1H-isoquinolin-2-yl)-2-hydroxy-propyl]-8-(trifluoromethyl)-2,3-dihydro-1,4-benzoxazepin-5-one;
(6) 2-[(2R)-3-(3,4-dihydro-1H-isoquinolin-2-yl)-2-hydroxy-propyl]-7-(trifluoromethoxy)-4,5-dihydro-3H-2-benzazepin-1-one;
(7) 2-[(2R)-3-(3,4-dihydro-1H-isoquinolin-2-yl)-2-hydroxy-propyl]-7-ethoxy-4,5-dihydro-3H-2-benzazepin-1-one;
(8) [2-[(2R)-3-(3,4-dihydro-1H-isoquinolin-2-yl)-2-hydroxy-propyl]-1-oxo-4,5-dihydro-3H-2-benzazepin-7-yl]trifluoromethanesulfonate;
(9) 2-[(2R)-3-(3,4-dihydro-1H-isoquinolin-2-yl)-2-hydroxy-propyl]-1-oxo-4,5-dihydro-3H-2-benzazepine-7-carbonitrile;
(10) 4-[(2R)-3-(3,4-dihydro-1H-isoquinolin-2-yl)-2-hydroxy-propyl]-8-methyl-2,3-dihydro-1,4-benzoxazepin-5-one;
(11) 4-[(2R)-3-(3,4-dihydro-1H-isoquinolin-2-yl)-2-hydroxy-propyl]-8-propyl-2,3-dihydro-1,4-benzoxazepin-5-one;
(12) 4-[(2R)-3-(3,4-dihydro-1H-isoquinolin-2-yl)-2-hydroxy-propyl]-8-isobutyl-2,3-dihydro-1,4-benzoxazepin-5-one;
(13) 4-[(2R)-3-(3,4-dihydro-1H-isoquinolin-2-yl)-2-hydroxy-propyl]-8-(3,3,3-trifluoropropyl)-2,3-dihydro-1,4-benzoxazepin-5-one;
(14) tert-Butyl 4-[4-[(2R)-3-(3,4-dihydro-1H-isoquinolin-2-yl)-2-hydroxy-propyl]-5-oxo-2,3-dihydro-1,4-benzoxazepin-8-yl]-3,6-dihydro-2H-pyridine-1-carboxylate;
(15) 4-[(2R)-3-(3,4-dihydro-1H-isoquinolin-2-yl)-2-hydroxy-propyl]-8-(1,2,3,6-tetrahydropyridin-4-yl)-2,3-dihydro-1,4-benzoxazepin-5-one;
(16) 8-(1-Acetyl-3,6-dihydro-2H-pyridin-4-yl)-4-[(2R)-3-(3,4-dihydro-1H-isoquinolin-2-yl)-2-hydroxy-propyl]-2,3-dihydro-1,4-benzoxazepin-5-one;
(17) 8-(1-Acetyl-4-piperidyl)-4-[(2R)-3-(3,4-dihydro-1H-isoquinolin-2-yl)-2-hydroxy-propyl]-2,3-dihydro-1,4-benzoxazepin-5-one;
(18) 4-[(2R)-3-(3,4-dihydro-1H-isoquinolin-2-yl)-2-hydroxy-propyl]-8-(1-ethyl-3,6-dihydro-2H-pyridin-4-yl)-2,3-dihydro-1,4-benzoxazepin-5-one;
(19) 4-[(2R)-3-(3,4-dihydro-1H-isoquinolin-2-yl)-2-hydroxy-propyl]-8-(1-isopropyl-3,6-dihydro-2H-pyridin-4-yl)-2,3-dihydro-1,4-benzoxazepin-5-one;
(20) 4-[(2R)-3-(3,4-dihydro-1H-isoquinolin-2-yl)-2-hydroxy-propyl]-8-(3,6-dihydro-2H-pyran-4-yl)-2,3-dihydro-1,4-benzoxazepin-5-one;
(21) 4-[(2R)-3-(3,4-dihydro-1H-isoquinolin-2-yl)-2-hydroxy-propyl]-8-tetrahydropyran-4-yl-2,3-dihydro-1,4-benzoxazepin-5-one;
(22) 4-[(2R)-3-(3,4-dihydro-1H-isoquinolin-2-yl)-2-hydroxy-propyl]-8-(morpholinomethyl)-2,3-dihydro-1,4-benzoxazepin-5-one;
(23) 4-[(2R)-3-(3,4-dihydro-1H-isoquinolin-2-yl)-2-hydroxy-propyl]-8-(1-piperidylmethyl)-2,3-dihydro-1,4-benzoxazepin-5-one;
(24) 4-[(2R)-3-(3,4-dihydro-1H-isoquinolin-2-yl)-2-hydroxy-propyl]-8-(pyrrolidin-1-ylmethyl)-2,3-dihydro-1,4-benzoxazepin-5-one;
(25) 4-[(2R)-3-(3,4-dihydro-1H-isoquinolin-2-yl)-2-hydroxy-propyl]-8-[(dimethylamino)methyl]-2,3-dihydro-1,4-benzoxazepin-5-one;
(26) 8-(Diethylaminomethyl)-4-[(2R)-3-(3,4-dihydro-1H-isoquinolin-2-yl)-2-hydroxy-propyl]-2,3-dihydro-1,4-benzoxazepin-5-one;
(27) 4-[(2R)-3-(3,4-dihydro-1H-isoquinolin-2-yl)-2-hydroxy-propyl]-8-[(4-methyl-1-piperidyl)methyl]-2,3-dihydro-1,4-benzoxazepin-5-one;
(28) 4-[(2R)-3-(3,4-dihydro-1H-isoquinolin-2-yl)-2-hydroxy-propyl]-8-(2-oxa-6-azaspiro[3.3]heptan-6-ylmethyl)-2,3-dihydro-1,4-benzoxazepin-5-one;
(29) 4-[(2R)-3-(3,4-dihydro-1H-isoquinolin-2-yl)-2-hydroxy-propyl]-8-[(3-methoxyazetidin-1-yl)methyl]-2,3-dihydro-1,4-benzoxazepin-5-one;
(30) 4-[(2R)-3-(3,4-dihydro-1H-isoquinolin-2-yl)-2-hydroxy-propyl]-8-[(2-methylmorpholin-4-yl)methyl]-2,3-dihydro-1,4-benzoxazepin-5-one;
(31) 8-[(4,4-difluoro-1-piperidyl)methyl]-4-[(2R)-3-(3,4-dihydro-1H-isoquinolin-2-yl)-2-hydroxy-propyl]-2,3-dihydro-1,4-benzoxazepin-5-one;
(32) 4-[(2R)-3-(3,4-dihydro-1H-isoquinolin-2-yl)-2-hydroxy-propyl]-8-[(4-fluoro-1-piperidyl)methyl]-2,3-dihydro-1,4-benzoxazepin-5-one;
(33) 4-[(2R)-3-(3,4-dihydro-1H-isoquinolin-2-yl)-2-hydroxy-propyl]-8-[(3,5-dimethyl-1-piperidyl)methyl]-2,3-dihydro-1,4-benzoxazepin-5-one;
(34) 8-(3,3a,4,5,6,6a-Hexahydro-1H-cyclopenta[c]pyrrol-2-ylmethyl)-4-[(2R)-3-(3,4-dihydro-1H-isoquinolin-2-yl)-2-hydroxy-propyl]-2,3-dihydro-1,4-benzoxazepin-5-one;
(35) 4-[(2R)-3-(3,4-dihydro-1H-isoquinolin-2-yl)-2-hydroxy-propyl]-8-[(3-hydroxypyrrolidin-1-yl)methyl]-2,3-dihydro-1,4-benzoxazepin-5-one;
(36) 4-[(2R)-3-(3,4-dihydro-1H-isoquinolin-2-yl)-2-hydroxy-propyl]-8-[[(2R)-2-methylpyrrolidin-1-yl]methyl]-2,3-dihydro-1,4-benzoxazepin-5-one;
(37) 4-[(2R)-3-(3,4-dihydro-1H-isoquinolin-2-yl)-2-hydroxy-propyl]-8-[[3-(hydroxymethyl)-1-piperidyl]methyl]-2,3-dihydro-1,4-benzoxazepin-5-one;
(38) 4-[(2R)-3-(3,4-dihydro-1H-isoquinolin-2-yl)-2-hydroxy-propyl]-8-[(4-methoxy-1-piperidyl)methyl]-2,3-dihydro-1,4-benzoxazepin-5-one;
(39) 4-[(2R)-3-(3,4-dihydro-1H-isoquinolin-2-yl)-2-hydroxy-propyl]-8-(1,4-oxazepan-4-ylmethyl)-2,3-dihydro-1,4-benzoxazepin-5-one;
(40) 4-[(2R)-3-(3,4-dihydro-1H-isoquinolin-2-yl)-2-hydroxy-propyl]-8-[(3-hydroxy-3-methyl-pyrrolidin-1-yl)methyl]-2,3-dihydro-1,4-benzoxazepin-5-one;
(41) 4-[(2R)-3-(3,4-dihydro-1H-isoquinolin-2-yl)-2-hydroxy-propyl]-8-[[3-(hydroxymethyl)morpholin-4-yl]methyl]-2,3-dihydro-1,4-benzoxazepin-5-one;
(42) 8-[(3,3-difluoro-1-piperidyl)methyl]-4-[(2R)-3-(3,4-dihydro-1H-isoquinolin-2-yl)-2-hydroxy-propyl]-2,3-dihydro-1,4-benzoxazepin-5-one;
(43) 4-[(2R)-3-(3,4-dihydro-1H-isoquinolin-2-yl)-2-hydroxy-propyl]-8-[(3-fluoro-1-piperidyl)methyl]-2,3-dihydro-1,4-benzoxazepin-5-one;
(44) 4-[(2R)-3-(3,4-dihydro-1H-isoquinolin-2-yl)-2-hydroxy-propyl]-8-[(3-hydroxy-1-piperidyl)methyl]-2,3-dihydro-1,4-benzoxazepin-5-one;
(45) 4-[(2R)-3-(3,4-dihydro-1H-isoquinolin-2-yl)-2-hydroxy-propyl]-8-[(4-hydroxy-1-piperidyl)methyl]-2,3-dihydro-1,4-benzoxazepin-5-one;
(46) 4-[(2R)-3-(3,4-dihydro-1H-isoquinolin-2-yl)-2-hydroxy-propyl]-8-[[(2S)-2-(hydroxymethyl)pyrrolidin-1-yl]methyl]-2,3-dihydro-1,4-benzoxazepin-5-one;
(47) 4-[(2R)-3-(3,4-dihydro-1H-isoquinolin-2-yl)-2-hydroxy-propyl]-8-[[(2R)-2-(hydroxymethyl)pyrrolidin-1-yl]methyl]-2,3-dihydro-1,4-benzoxazepin-5-one;
(48) 4-[(2R)-3-(3,4-dihydro-1H-isoquinolin-2-yl)-2-hydroxy-propyl]-8-[(2-ethylpyrrolidin-1-yl)methyl]-2,3-dihydro-1,4-benzoxazepin-5-one;
(49) 4-[(2R)-3-(3,4-dihydro-1H-isoquinolin-2-yl)-2-hydroxy-propyl]-8-[[(2S)-2-(methoxymethyl)pyrrolidin-1-yl]methyl]-2,3-dihydro-1,4-benzoxazepin-5-one;
(50) 4-[(2R)-3-(3,4-dihydro-1H-isoquinolin-2-yl)-2-hydroxy-propyl]-8-[[(2R)-2-(methoxymethyl)pyrrolidin-1-yl]methyl]-2,3-dihydro-1,4-benzoxazepin-5-one;
(51) 4-[(2R)-3-(3,4-dihydro-1H-isoquinolin-2-yl)-2-hydroxy-propyl]-8-[(3-fluoropyrrolidin-1-yl)methyl]-2,3-dihydro-1,4-benzoxazepin-5-one;
(52) 8-[(3,3-difluoropyrrolidin-1-yl)methyl]-4-[(2R)-3-(3,4-dihydro-1H-isoquinolin-2-yl)-2-hydroxy-propyl]-2,3-dihydro-1,4-benzoxazepin-5-one;
(53) 8-[(4-acetylpiperazin-1-yl)methyl]-4-[(2R)-3-(3,4-dihydro-1H-isoquinolin-2-yl)-2-hydroxy-propyl]-2,3-dihydro-1,4-benzoxazepin-5-one;
(54) 4-[(2R)-3-(3,4-dihydro-1H-isoquinolin-2-yl)-2-hydroxy-propyl]-8-[(4-methylpiperazin-1-yl)methyl]-2,3-dihydro-1,4-benzoxazepin-5-one;
(55) 4-[(2R)-3-(3,4-dihydro-1H-isoquinolin-2-yl)-2-hydroxy-propyl]-8-(2-oxa-5-azabicyclo[2.2.1]heptan-5-ylmethyl)-2,3-dihydro-1,4-benzoxazepin-5-one;
(56) 4-[(2R)-3-(3,4-dihydro-1H-isoquinolin-2-yl)-2-hydroxy-propyl]-8-(3-pyridylmethoxy)-2,3-dihydro-1,4-benzoxazepin-5-one;
(57) 4-[(2R)-3-(3,4-dihydro-1H-isoquinolin-2-yl)-2-hydroxy-propyl]-8-(4-pyridylmethoxy)-2,3-dihydro-1,4-benzoxazepin-5-one;
(58) 4-[(2R)-3-(3,4-dihydro-1H-isoquinolin-2-yl)-2-hydroxy-propyl]-8-[(2-fluoro-4-pyridyl)methoxy]-2,3-dihydro-1,4-benzoxazepin-5-one;
(59) 8-[(2,6-Dichloro-4-pyridyl)methoxy]-4-[(2R)-3-(3,4-dihydro-1H-isoquinolin-2-yl)-2-hydroxy-propyl]-2,3-dihydro-1,4-benzoxazepin-5-one;
(60) 8-[(2,3-Difluoro-4-pyridyl)methoxy]-4-[(2R)-3-(3,4-dihydro-1H-isoquinolin-2-yl)-2-hydroxy-propyl]-2,3-dihydro-1,4-benzoxazepin-5-one;
(61) 4-[(2R)-3-(3,4-dihydro-1H-isoquinolin-2-yl)-2-hydroxy-propyl]-8-[(6-fluoro-3-pyridyl)methoxy]-2,3-dihydro-1,4-benzoxazepin-5-one;
(62) 4-[(2R)-3-(3,4-dihydro-1H-isoquinolin-2-yl)-2-hydroxy-propyl]-8-[(3-fluoro-4-pyridyl)methoxy]-2,3-dihydro-1,4-benzoxazepin-5-one;
(63) 4-[(2R)-3-(3,4-dihydro-1H-isoquinolin-2-yl)-2-hydroxy-propyl]-8-[(5-fluoro-2-pyridyl)methoxy]-2,3-dihydro-1,4-benzoxazepin-5-one;
(64) 4-[(2R)-3-(3,4-dihydro-1H-isoquinolin-2-yl)-2-hydroxy-propyl]-8-tetrahydropyran-4-yloxy-2,3-dihydro-1,4-benzoxazepin-5-one;
(65) 4-[(2R)-3-(3,4-dihydro-1H-isoquinolin-2-yl)-2-hydroxy-propyl]-8-(tetrahydropyran-2-ylmethoxy)-2,3-dihydro-1,4-benzoxazepin-5-one;
(66) 8-(Cyclohexylmethoxy)-4-[(2R)-3-(3,4-dihydro-1H-isoquinolin-2-yl)-2-hydroxy-propyl]-2,3-dihydro-1,4-benzoxazepin-5-one;
(67) 4-[(2R)-3-(3,4-dihydro-1H-isoquinolin-2-yl)-2-hydroxy-propyl]-8-(tetrahydrofuran-2-ylmethoxy)-2,3-dihydro-1,4-benzoxazepin-5-one;
(68) 4-[(2R)-3-(3,4-dihydro-1H-isoquinolin-2-yl)-2-hydroxy-propyl]-8-[(1-methyl-3-piperidyl)oxy]-2,3-dihydro-1,4-benzoxazepin-5-one;
(69) 4-[(2R)-3-(3,4-dihydro-1H-isoquinolin-2-yl)-2-hydroxy-propyl]-8-[(1-ethyl-3-piperidyl)oxy]-2,3-dihydro-1,4-benzoxazepin-5-one;
(70) 8-[(1-Acetyl-4-piperidyl)methoxy]-4-[(2R)-3-(3,4-dihydro-1H-isoquinolin-2-yl)-2-hydroxy-propyl]-2,3-dihydro-1,4-benzoxazepin-5-one;
(71) 4-[(2R)-3-(3,4-dihydro-1H-isoquinolin-2-yl)-2-hydroxy-propyl]-8-(2,2,2-trifluoroethoxy)-2,3-dihydro-1,4-benzoxazepin-5-one;
(72) 4-[(2R)-3-(3,4-dihydro-1H-isoquinolin-2-yl)-2-hydroxy-propyl]-8-[2-(dimethylamino)ethoxy]-2,3-dihydro-1,4-benzoxazepin-5-one;
(73) 4-[(2R)-3-(3,4-dihydro-1H-isoquinolin-2-yl)-2-hydroxy-propyl]-8-(2-morpholinoethoxy)-2,3-dihydro-1,4-benzoxazepin-5-one;
(74) 4-[(2R)-3-(3,4-dihydro-1H-isoquinolin-2-yl)-2-hydroxy-propyl]-8-[2-(2-oxopyrrolidin-1-yl)ethoxy]-2,3-dihydro-1,4-benzoxazepin-5-one;
(75) 4-[(2R)-3-(3,4-dihydro-1H-isoquinolin-2-yl)-2-hydroxy-propyl]-8-(2-pyridyloxy)-2,3-dihydro-1,4-benzoxazepin-5-one;
(76) 4-[(2R)-3-(3,4-dihydro-1H-isoquinolin-2-yl)-2-hydroxy-propyl]-8-[(6-oxo-3-piperidyl)oxy]-2,3-dihydro-1,4-benzoxazepin-5-one;
(77) tert-Butyl 4-[[4-[(2R)-3-(3,4-dihydro-1H-isoquinolin-2-yl)-2-hydroxy-propyl]-5-oxo-2,3-dihydro-1,4-benzoxazepin-8-yl]oxy]piperidine-1-carboxylate;
(78) 8-[(1-Acetyl-4-piperidyl)oxy]-4-[(2R)-3-(3,4-dihydro-1H-isoquinolin-2-yl)-2-hydroxy-propyl]-2,3-dihydro-1,4-benzoxazepin-5-one;
(79) 8-[(1-Acetyl-3-piperidyl)oxy]-4-[(2R)-3-(3,4-dihydro-1H-isoquinolin-2-yl)-2-hydroxy-propyl]-2,3-dihydro-1,4-benzoxazepin-5-one;
(80) 8-(1-Acetylpyrrolidin-3-yl)oxy-4-[(2R)-3-(3,4-dihydro-1H-isoquinolin-2-yl)-2-hydroxy-propyl]-2,3-dihydro-1,4-benzoxazepin-5-one;
(81) 8-[(3R)-1-acetylpyrrolidin-3-yl]oxy-4-[(2R)-3-(3,4-dihydro-1H-isoquinolin-2-yl)-2-hydroxy-propyl]-2,3-dihydro-1,4-benzoxazepin-5-one;
(82) 8-[(3S)-1-acetylpyrrolidin-3-yl]oxy-4-[(2R)-3-(3,4-dihydro-1H-isoquinolin-2-yl)-2-hydroxy-propyl]-2,3-dihydro-1,4-benzoxazepin-5-one;
(83) 8-(1-Acetylazetidin-3-yl)oxy-4-[(2R)-3-(3,4-dihydro-1H-isoquinolin-2-yl)-2-hydroxy-propyl]-2,3-dihydro-1,4-benzoxazepin-5-one;
(84) 4-[(2R)-3-(3,4-dihydro-1H-isoquinolin-2-yl)-2-hydroxy-propyl]-8-(1-propanoylazetidin-3-yl)oxy-2,3-dihydro-1,4-benzoxazepin-5-one;
(85) 8-[1-(Cyclopropanecarbonyl)azetidin-3-yl]oxy-4-[(2R)-3-(3,4-dihydro-1H-isoquinolin-2-yl)-2-hydroxy-propyl]-2,3-dihydro-1,4-benzoxazepin-5-one;
(86) Methyl 3-[[4-[(2R)-3-(3,4-dihydro-1H-isoquinolin-2-yl)-2-hydroxy-propyl]-5-oxo-2,3-dihydro-1,4-benzoxazepin-8-yl]oxy]azetidine-1-carboxylate;
(87) 4-[(2R)-3-(3,4-dihydro-1H-isoquinolin-2-yl)-2-hydroxy-propyl]-8-[(1-methyl-4-piperidyl)oxy]-2,3-dihydro-1,4-benzoxazepin-5-one;
(88) 4-[(2R)-3-(3,4-dihydro-1H-isoquinolin-2-yl)-2-hydroxy-propyl]-8-[(1-ethyl-4-piperidyl)oxy]-2,3-dihydro-1,4-benzoxazepin-5-one;
(89) 4-[(2R)-3-(3,4-dihydro-1H-isoquinolin-2-yl)-2-hydroxy-propyl]-8-[(1-isopropyl-4-piperidyl)oxy]-2,3-dihydro-1,4-benzoxazepin-5-one;
(90) 8-[(1-Cyclopropyl-4-piperidyl)oxy]-4-[(2R)-3-(3,4-dihydro-1H-isoquinolin-2-yl)-2-hydroxy-propyl]-2,3-dihydro-1,4-benzoxazepine-5-one;
(91) 8-[(1-Cyclobutyl-4-piperidyl)oxy]-4-[(2R)-3-(3,4-dihydro-1H-isoquinolin-2-yl)-2-hydroxy-propyl]-2,3-dihydro-1,4-benzoxazepin-5-one;
(92) 4-[(2R)-3-(3,4-dihydro-1H-isoquinolin-2-yl)-2-hydroxy-propyl]-8-[[1-(oxetan-3-yl)-4-piperidyl]oxy]-2,3-dihydro-1,4-benzoxazepin-5-one;
(93) 4-[(2R)-3-(3,4-dihydro-1H-isoquinolin-2-yl)-2-hydroxy-propyl]-8-(1-methylazetidin-3-yl)oxy-2,3-dihydro-1,4-benzoxazepin-5-one;
(94) 4-[(2R)-3-(3,4-dihydro-1H-isoquinolin-2-yl)-2-hydroxy-propyl]-8-(1-ethylazetidin-3-yl)oxy-2,3-dihydro-1,4-benzoxazepin-5-one;
(95) 4-[(2R)-3-(3,4-dihydro-1H-isoquinolin-2-yl)-2-hydroxy-propyl]-8-(1-isopropylazetidin-3-yl)oxy-2,3-dihydro-1,4-benzoxazepin-5-one;
(96) 4-[(2R)-3-(3,4-dihydro-1H-isoquinolin-2-yl)-2-hydroxy-propyl]-8-[1-(oxetan-3-yl)azetidin-3-yl]oxy-2,3-dihydro-1,4-benzoxazepin-5-one;
(97) 4-[(2R)-3-(3,4-dihydro-1H-isoquinolin-2-yl)-2-hydroxy-propyl]-8-[(1-ethylazetidin-3-yl)methoxy]-2,3-dihydro-1,4-benzoxazepin-5-one;
(98) 4-[(2R)-3-(3,4-dihydro-1H-isoquinolin-2-yl)-2-hydroxy-propyl]-8-(1-methylpyrrolidin-3-yl)oxy-2,3-dihydro-1,4-benzoxazepin-5-one;
(99) 4-[(2R)-3-(3,4-dihydro-1H-isoquinolin-2-yl)-2-hydroxy-propyl]-8-[(3R)-1-ethylpyrrolidin-3-yl]oxy-2,3-dihydro-1,4-benzoxazepin-5-one;
(100) 4-[(2R)-3-(3,4-dihydro-1H-isoquinolin-2-yl)-2-hydroxy-propyl]-8-(2-pyridyloxy)-2,3-dihydro-1,4-benzoxazepin-5-one;
(101) 4-[(2R)-3-(3,4-dihydro-1H-isoquinolin-2-yl)-2-hydroxy-propyl]-8-[[(2S)-1-isopropylpyrrolidin-2-yl]methoxy]-2,3-dihydro-1,4-benzoxazepin-5-one;
(102) 4-[(2R)-3-(3,4-dihydro-1H-isoquinolin-2-yl)-2-hydroxy-propyl]-8-[(1-methyl-4-piperidyl)methoxy]-2,3-dihydro-1,4-benzoxazepin-5-one;
(103) 4-[(2R)-3-(3,4-dihydro-1H-isoquinolin-2-yl)-2-hydroxy-propyl]-8-[(1-ethyl-4-piperidyl)methoxy]-2,3-dihydro-1,4-benzoxazepin-5-one;
(104) 4-[(2R)-3-(3,4-dihydro-1H-isoquinolin-2-yl)-2-hydroxy-propyl]-8-[(4-methylmorpholin-2-yl)methoxy]-2,3-dihydro-1,4-benzoxazepin-5-one;
(105) 4-[(2R)-3-(3,4-dihydro-1H-isoquinolin-2-yl)-2-hydroxy-propyl]-8-[(4-ethylmorpholin-2-yl)methoxy]-2,3-dihydro-1,4-benzoxazepin-5-one;
(106) 4-[(2R)-3-(3,4-dihydro-1H-isoquinolin-2-yl)-2-hydroxy-propyl]-8-[(1-methylpyrrolidin-3-yl)methoxy]-2,3-dihydro-1,4-benzoxazepin-5-one;
(107) 4-[(2R)-3-(3,4-dihydro-1H-isoquinolin-2-yl)-2-hydroxy-propyl]-8-[(1-methyl-3-piperidyl)methoxy]-2,3-dihydro-1,4-benzoxazepin-5-one;
(108) 4-[(2R)-3-(3,4-dihydro-1H-isoquinolin-2-yl)-2-hydroxy-propyl]-6-fluoro-8-(morpholinomethyl)-2,3-dihydro-1,4-benzoxazepin-5-one;
(109) 4-[(2R)-3-(3,4-dihydro-1H-isoquinolin-2-yl)-2-hydroxy-propyl]-6-fluoro-8-[(1-methyl-4-piperidyl)oxy]-2,3-dihydro-1,4-benzoxazepin-5-one;
(110) 4-[(2R)-3-(3,4-dihydro-1H-isoquinolin-2-yl)-2-hydroxy-propyl]-8-[(1-ethyl-4-piperidyl)oxy]-2,2-dimethyl-3H-1,4-benzoxazepin-5-one;
(111) 4-[(2R)-3-(3,4-dihydro-1H-isoquinolin-2-yl)-2-hydroxy-propyl]-2,2-dimethyl-8-(morpholinomethyl)-3H-1,4-benzoxazepin-5-one;
(112) 4-[(2R)-3-(3,4-dihydro-1H-isoquinolin-2-yl)-2-hydroxy-propyl]-2,2-dimethyl-8-[(1-methyl-4-piperidyl)oxy]-3H-1,4-benzoxazepin-5-one;
(113) 4-[(2R)-3-(3,4-dihydro-1H-isoquinolin-2-yl)-2-hydroxy-propyl]-2,2-dimethyl-8-(4-pyridylmethoxy)-3H-1,4-benzoxazepin-5-one;
(114) 8-(1-Acetylazetidin-3-yl)oxy-4-[(2R)-3-(3,4-dihydro-1H-isoquinolin-2-yl)-2-hydroxy-propyl]-2,2-dimethyl-3H-1,4-benzoxazepin-5-one;
(115) 4-[(2R)-3-(3,4-dihydro-1H-isoquinolin-2-yl)-2-hydroxy-propyl]-1-methyl-8-(morpholinomethyl)-2,3-dihydro-1,4-benzodiazepin-5-one;
(116) 4-[(2R)-3-(3,4-dihydro-1H-isoquinolin-2-yl)-2-hydroxy-propyl]-1-methyl-8-(3,3,3-trifluoropropyl)-2,3-dihydro-1,4-benzodiazepin-5-one;
(117) 8-(Cyclohexylmethyl)-4-[(2R)-3-(3,4-dihydro-1H-isoquinolin-2-yl)-2-hydroxy-propyl]-1-methyl-2,3-dihydro-1,4-benzodiazepin-5-one;
(118) 8-(Cyclohexen-1-yl)-4-[(2R)-3-(3,4-dihydro-1H-isoquinolin-2-yl)-2-hydroxy-propyl]-1-methyl-2,3-dihydro-1,4-benzodiazepin-5-one;
(119) 4-[(2R)-3-(3,4-dihydro-1H-isoquinolin-2-yl)-2-hydroxy-propyl]-1-methyl-8-[4-(trifluoromethyl)cyclohexen-1-yl]-2,3-dihydro-1,4-benzodiazepin-5-one;
(120) tert-Butyl 4-[4-[(2R)-3-(3,4-dihydro-1H-isoquinolin-2-yl)-2-hydroxy-propyl]-1-methyl-5-oxo-2,3-dihydro-1,4-benzodiazepin-8-yl]-3,6-dihydro-2H-pyridine-1-carboxylate;
(121) 8-[(1-Acetyl-4-piperidyl)oxy]-4-[(2R)-3-(3,4-dihydro-1H-isoquinolin-2-yl)-2-hydroxy-propyl]-1-methyl-2,3-dihydro-1,4-benzodiazepin-5-one;
(122) 8-(1-Acetylazetidin-3-yl)oxy-4-[(2R)-3-(3,4-dihydro-1H-isoquinolin-2-yl)-2-hydroxy-propyl]-1-methyl-2,3-dihydro-1,4-benzodiazepin-5-one;
(123) 8-[(3R)-1-acetylpyrrolidin-3-yl]oxy-4-[(2R)-3-(3,4-dihydro-1H-isoquinolin-2-yl)-2-hydroxy-propyl]-1-methyl-2,3-dihydro-1,4-benzodiazepin-5-one;
(124) 8-[(3S)-1-acetylpyrrolidin-3-yl]oxy-4-[(2R)-3-(3,4-dihydro-1H-isoquinolin-2-yl)-2-hydroxy-propyl]-1-methyl-2,3-dihydro-1,4-benzodiazepin-5-one;
(125) 4-[(2R)-3-(3,4-dihydro-1H-isoquinolin-2-yl)-2-hydroxy-propyl]-1-methyl-8-[(1-methyl-4-piperidyl)oxy]-2,3-dihydro-1,4-benzodiazepin-5-one;
(126) 4-[(2R)-3-(3,4-dihydro-1H-isoquinolin-2-yl)-2-hydroxy-propyl]-8-[(1-ethyl-4-piperidyl)oxy]-1-methyl-2,3-dihydro-1,4-benzodiazepin-5-one;
(127) 8-[(1-Cyclobutyl-4-piperidyl)oxy]-4-[(2R)-3-(3,4-dihydro-1H-isoquinolin-2-yl)-2-hydroxy-propyl]-1-methyl-2,3-dihydro-1,4-benzodiazepin-5-one;
(128) 4-[(2R)-3-(3,4-dihydro-1H-isoquinolin-2-yl)-2-hydroxy-propyl]-1-methyl-8-[(1-tetrahydrofuran-3-yl-4-piperidyl)oxy]-2,3-dihydro-1,4-benzodiazepin-5-one;
(129) 4-[(2R)-3-(3,4-dihydro-1H-isoquinolin-2-yl)-2-hydroxy-propyl]-1-methyl-8-[[1-(oxetan-3-yl)-4-piperidyl]oxy]-2,3-dihydro-1,4-benzodiazepin-5-one;
(130) 4-[(2R)-3-(3,4-dihydro-1H-isoquinolin-2-yl)-2-hydroxy-propyl]-1-methyl-8-[(1-methyl-3-piperidyl)oxy]-2,3-dihydro-1,4-benzodiazepin-5-one;
(131) 4-[(2R)-3-(3,4-dihydro-1H-isoquinolin-2-yl)-2-hydroxy-propyl]-1-methyl-8-[(1-methylpyrrolidin-3-yl)methoxy]-2,3-dihydro-1,4-benzodiazepin-5-one;
(132) 4-[(2R)-3-(3,4-dihydro-1H-isoquinolin-2-yl)-2-hydroxy-propyl]-1-methyl-8-(1-methylpyrrolidin-3-yl)oxy-2,3-dihydro-1,4-benzodiazepin-5-one;
(133) 4-[(2R)-3-(3,4-dihydro-1H-isoquinolin-2-yl)-2-hydroxy-propyl]-1-methyl-8-[(1-methyl-3-piperidyl)methoxy]-2,3-dihydro-1,4-benzodiazepin-5-one;
(134) 4-[(2R)-3-(3,4-dihydro-1H-isoquinolin-2-yl)-2-hydroxy-propyl]-8-[(3-fluoro-4-pyridyl)methoxy]-1-methyl-2,3-dihydro-1,4-benzodiazepin-5-one;
(135) 4-[(2R)-3-(3,4-dihydro-1H-isoquinolin-2-yl)-2-hydroxy-propyl]-8-[(3-fluoro-4-piperidyl)oxy]-1-methyl-2,3-dihydro-1,4-benzodiazepin-5-one 2 hydrochloride;
(136) 4-[(2R)-3-(3,4-dihydro-1H-isoquinolin-2-yl)-2-hydroxy-propyl]-8-[(1-ethyl-3-fluoro-4-piperidyl)oxy]-1-methyl-2,3-dihydro-1,4-benzodiazepin-5-one;
(137) 4-[(2R)-3-(3,4-dihydro-1H-isoquinolin-2-yl)-2-hydroxy-propyl]-8-[(8-ethyl-8-azabicyclo[3.2.1]octan-3-yl)oxy]-1-methyl-2,3-dihydro-1,4-benzodiazepin-5-one;
(138) 4-[(2R)-3-(3,4-dihydro-1H-isoquinolin-2-yl)-2-hydroxy-propyl]-1-methyl-8-[(2-methyl-4-piperidyl)oxy]-2,3-dihydro-1,4-benzodiazepin-5-one 2 hydrochloride;
(139) 4-[(2R)-3-(3,4-dihydro-1H-isoquinolin-2-yl)-2-hydroxy-propyl]-8-[(5-fluoro-2-pyridyl)methoxy]-1-methyl-2,3-dihydro-1,4-benzodiazepin-5-one;
(140) 4-[(2R)-3-(3,4-dihydro-1H-isoquinolin-2-yl)-2-hydroxypropyl]-2-methyl-8-(4-piperidyloxy)-2,3-dihydro-1,4-benzoxazepin-5-one 2 hydrochloride;
(141) 4-[(2R)-3-(3,4-dihydro-1H-isoquinolin-2-yl)-2-hydroxypropyl]-2-methyl-8-[(1-methyl-4-piperidyl)oxy]-2,3-dihydro-1,4-benzoxazepin-5-one;
(142) (2R)-8-(1-acetyazetidin-3-yl)oxy-4-[(2R)-3-(3,4-dihydro-1H-isoquinolin-2-yl)-2-hydroxypropyl]-2-methyl-2,3-dihydro-1,4-benzoxazepin-5-one;
(143) (2R)-4-[(2R)-3-(3,4-dihydro-1H-isoquinolin-2-yl)-2-hydroxypropyl]-2-methyl-8-[(1-methyl-4-piperidyl)oxy]-2,3-dihydro-1,4-benzoxazepin-5-one;
(144) 4-[(2R)-3-(3,4-dihydro-1H-isoquinolin-2-yl)-2-hydroxypropyl]-2-methyl-8-[(1-methyl-4-piperidyl)oxy]-2,3-dihydropyrido[3,2-f][1,4]oxazepin-5-one;
(145) 4-[(2R)-3-(3,4-dihydro-1H-isoquinolin-2-yl)-2-hydroxypropyl]-2,2-dimethyl-8-[(1-methyl-4-piperidyl)oxy]-3H-pyrido[3,2-f][1,4]oxazepin-5-one;
(146) (2S)-4-[(2R)-3-(3,4-dihydro-1H-isoquinolin-2-yl)-2-hydroxypropyl]-2-methyl-8-[(1-methyl-4-piperidyl)oxy]-2,3-dihydro-1,4-benzoxazepin-5-one;
(147) 4-[3-(3,4-dihydro-1H-isoquinolin-2-yl)-2-hydroxy-propyl]-2,3-dihydro-1,4-benzoxazepin-5-one;
(148) 2-[3-(3,4-dihydro-1H-isoquinolin-2-yl)-2-hydroxy-propyl]-4,5-dihydro-3H-2-benzazepin-1-one;
(149) 4-[(2R)-3-(3,4-dihydro-1H-isoquinolin-2-yl)-2-hydroxy-propyl]-2,3-dihydro-1,4-benzoxazepin-5-one;
(150) 4-[(2R)-3-(3,4-dihydro-1H-isoquinolin-2-yl)-2-hydroxy-propyl]-8-fluoro-2,3-dihydro-1,4-benzoxazepin-5-one;
(151) 8-Bromo-4-[(2R)-3-(3,4-dihydro-1H-isoquinolin-2-yl)-2-hydroxy-propyl]-2,3-dihydro-1,4-benzoxazepin-5-one;
(152) 4-[(2R)-3-(3,4-dihydro-1H-isoquinolin-2-yl)-2-hydroxy-propyl]-2,3-dihydropyrido[4,3-f][1,4]oxazepin-5-one;
(153) 8-Chloro-4-[(2R)-3-(3,4-dihydro-1H-isoquinolin-2-yl)-2-hydroxy-propyl]-2,3-dihydropyrido[3,2-f][1,4]oxazepin-5-one;
(154) 7-Chloro-2-[(2R)-3-(3,4-dihydro-1H-isoquinolin-2-yl)-2-hydroxy-propyl]-4,5-dihydro-3H-2-benzazepin-1-one;
(155) 7-Bromo-2-[(2R)-3-(3,4-dihydro-1H-isoquinolin-2-yl)-2-hydroxy-propyl]-4,5-dihydro-3H-2-benzazepin-1-one;
(156) 8-Bromo-4-[(2R)-3-(3,4-dihydro-1H-isoquinolin-2-yl)-2-hydroxy-propyl]-1-methyl-2,3-dihydro-1,4-benzodiazepin-5-one;
(157) 8-Bromo-4-[(2R)-3-(3,4-dihydro-1H-isoquinolin-2-yl)-2-hydroxy-propyl]-1-ethyl-2,3-dihydro-1,4-benzodiazepin-5-one;
(158) 8-Bromo-4-[(2R)-3-(3,4-dihydro-1H-isoquinolin-2-yl)-2-hydroxy-propyl]-1-ethyl-3H-1,4-benzodiazepine-2,5-dione;
(159) 2-[4-[[4-(2R)-3-(3,4,-dihydro-1H-isoquinolin-2-yl)-2-hydroxy-propyl]-5-oxo-2,3,-dihydro-1,4,-benzoxazepin-8-yl]-1-piperidyl]acetonitrile;
(160) 8-[[1-(2,2-difluoroacetyl)-4-piperidyl]oxy]-4-[(2R)-3-(3,4-dihydro-1H-isoquinolin-2-yl)-2-hydroxy-propyl]-2,3-dihydro-1,4-benzoxazepin-5-one;
(161) 8-[1-(2,2-difluoroacetyl)azetidin-3-yl]oxy-4-[(2R)-3-(3,4-dihydro-1H-isoquinolin-2-yl)-2-hydroxy-propyl]-2,3-dihydro-1,4-benzoxazepin-5-one;
(162) 4-[(2R)-3-(3,4-dihydro-1H-isoquinolin-2-yl)-2-hydroxy-propyl]-8-[[1-(2-hydroxyethyl)-4-piperidyl]oxy]-2,3-dihydro-1,4-benzoxazepin-5-one;
(163) 4-[(2R)-3-(3,4-dihydro-1H-isoquinolin-2-yl)-2-hydroxy-propyl]-8-[[1-(2-fluoroethyl)-4-piperidyl]oxy]-2,3-dihydro-1,4-benzoxazepin-5-one;
(164) 4-[(2R)-3-(3,4-dihydro-1H-isoquinolin-2-yl)-2-hydroxy-propyl]-8-[[1-(2-fluoroethyl)-4-piperidyl]oxy]-2,3-dihydropyrido[3,2-f][1,4]oxazepin-5-one;
(165) 4-[(2R)-3-(3,4-dihydro-1H-isoquinolin-2-yl)-2-hydroxypropyl]-8-[[1-[(3-methyloxetan-3-yl)methyl]-4-piperidyl]oxy]-2,3-dihydro-1,4-benzoxazepin-5-one;
(166) 4-[[4-[(2R)-3-(3,4-dihydro-1H-isoquinolin-2-yl)-2-hydroxy-propyl]-5-oxo-2,3-dihydro-1,4-benzoazepin-8-yl]oxy]piperidine-1-carbonitrile;
(167) 8-[(4-acetylpiperazin-1-yl)methyl]-4-[(2R)-3-(3,4-dihydro-1H-isoquinolin-2-yl)-2-hydroxy-propyl]-2,2-dimethyl-3H-1,4-benzoxazepin-5-one;
(168) 3-[[4-[(2R)-3-(3,4-dihydro-1H-isoquinolin-2-yl)-2-hydroxy-propyl]-2,2-dimethyl-5-oxo-3H-1,4-benzoxazepin-8-yl]oxy]azetidine-1-carbaldehyde;
(169) 4-[[4-[(2R)-3-(3,4-dihydro-1H-isoquinolin-2-yl)-2-hydroxy-propyl]-2,2-dimethyl-5-oxo-3H-1,4-benzoxazepin-8-yl]methyl]piperazine-1-carbaldehyde;
(170) 4-[(2R)-3-(3,4-dihydro-1H-isoquinolin-2-yl)-2-hydroxy-propyl]-2,2-dimethyl-8-(2-oxa-6-azaspiro[3.3]heptan-6-ylmethyl)-3H-1,4-benzoxazepin-5-one;
(171) 4-[(2R)-3-(3,4-dihydro-1H-isoquinolin-2-yl)-2-hydroxy-propyl]-8-[(8-ethyl-8-azabicyclo[3.2.1]octan-3-yl)oxy]-2,3-dihydro-1,4-benzoxazepin-5-one;
(172) 4-[(2R)-3-(3,4-dihydro-1H-isoquinolin-2-yl)-2-hydroxy-propyl]-8-[(2-ethyl-2-azaspiro[3.3]heptan-6-yl)oxy]-2,3-dihydro-1,4-benzoxazepin-5-one;
(173) 8-[(8-Acetyl-8-azabicyclo[3.2.1]octan-3-yl)oxy]-4-[(2R)-3-(3,4-dihydro-1H-isoquinolin-2-yl)-2-hydroxy-propyl]-2,3-dihydro-1,4-benzoxazepin-5-one;
(174) 8-[(2-Acetyl-2-azaspiro[3.3]heptan-6-yl)oxy]-4-[(2R)-3-(3,4-dihydro-1H-isoquinolin-2-yl)-2-hydroxy-propyl]-2,3-dihydro-1,4-benzoxazepin-5-one;
(175) 4-[(2R)-3-(3,4-dihydro-1H-isoquinolin-2-yl)-2-hydroxy-propyl]-8-(pyrrolidine-1-carbonyl)-2,3-dihydro-1,4-benzoxazepin-5-one;
(176) 4-[(2R)-3-(3,4-dihydro-1H-isoquinolin-2-yl)-2-hydroxy-propyl]-8-(piperidine-1-carbonyl)-2,3-dihydro-1,4-benzoxazepin-5-one;
(177) 8-(3,3-Difluoropyrrolidine-1-carbonyl)-4-[(2R)-3-(3,4-dihydro-1H-isoquinolin-2-yl)-2-hydroxy-propyl]-2,3-dihydro-1,4-benzoxazepin-5-one;
(178) 4-[(2R)-3-(3,4-dihydro-1H-isoquinolin-2-yl)-2-hydroxy-propyl]-8-(4-methylpiperazine-1-carbonyl)-2,3-dihydro-1,4-benzoxazepin-5-one;
(179) 4-[(2R)-3-(3,4-dihydro-1H-isoquinolin-2-yl)-2-hydroxy-propyl]-2,2-dimethyl-8-(pyrrolidin-1-ylmethyl)-3H-1,4-benzoxazepin-5-one;
(180) 8-[(1-Acetyl-4-piperidyl)oxy]-4-[(2R)-3-(3,4-dihydro-1H-isoquinolin-2-yl)-2-hydroxy-propyl]-2,2-dimethyl-3H-1,4-benzoxazepin-5-one;
(181) 4-[[4-[(2R)-3-(3,4-dihydro-1H-isoquinolin-2-yl)-2-hydroxy-propyl]-2,2-dimethyl-5-oxo-3H-1,4-benzoxazepin-8-yl]oxy]piperidine-1-carbaldehyde;
(182) 4-[(2R)-3-(3,4-dihydro-1H-isoquinolin-2-yl)-2-hydroxy-propyl]-2,2-dimethyl-8-[[4-(oxetan-3-yl)piperazin-1-yl]methyl]-3H-1,4-benzoxazepin-5-one;
(183) 4-[(2R)-3-(3,4-dihydro-1H-isoquinolin-2-yl)-2-hydroxy-propyl]-2,2-dimethyl-8-(2-oxa-7-azaspiro[3.4]octan-7-ylmethyl)-3H-1,4-benzoxazapin-5-one;
(184) 4-[(2R)-3-(3,4-dihydro-1H-isoquinolin-2-yl)-2-hydroxy-propyl]-8-(4-fluoropiperidine-1-carbonyl)-2,3-dihydro-1,4-benzoxazepin-5-one;
(185) 4-[(2R)-3-(3,4-dihydro-1H-isoquinolin-2-yl)-2-hydroxy-propyl]-8-[3-(trifluoromethyl)piperidine-1-carbonyl]-2,3-dihydro-1,4-benzoxazepin-5-one;
(186) 4-[(2R)-3-(3,4-dihydro-1H-isoquinolin-2-yl)-2-hydroxy-propyl]-8-[4-(trifluoromethyl)piperidine-1-carbonyl]-2,3-dihydro-1,4-benzoxazepin-5-one;
(187) 4-[(2R)-3-(3,4-dihydro-1H-isoquinolin-2-yl)-2-hydroxy-propyl]-8-(2,6-dimethylmorpholine-4-carbonyl)-2,3-dihydro-1,4-benzoxazepin-5-one;
(188) 8-(2-Azabicyclo[2.2.1]heptane-2-carbonyl)-4-[(2R)-3-(3,4-dihydro-1H-isoquinolin-2-yl)-2-hydroxy-propyl]-2,3-dihydro-1,4-benzoxazepin-5-one;
(189) 4-[(2R)-3-(3,4-dihydro-1H-isoquinolin-2-yl)-2-hydroxy-propyl]-8-(3-oxa-8-azabicyclo[3.2.1]octane-8-carbonyl)-2,3-dihydro-1,4-benzoxazepin-5-one;
(190) 4-[(2R)-3-(3,4-dihydro-1H-isoquinolin-2-yl)-2-hydroxy-propyl]-8-(2-methylmorpholine-4-carbonyl)-2,3-dihydro-1,4-benzoxazepin-5-one;
(191) 4-[(2R)-3-(3,4-dihydro-1H-isoquinolin-2-yl)-2-hydroxy-propyl]-8-(3-methylmorpholine-4-carbonyl)-2,3-dihydro-1,4-benzoxazepin-5-one;
(192) 4-[(2R)-3-(3,4-dihydro-1H-isoquinolin-2-yl)-2-hydroxy-propyl]-2,2-dimethyl-8-(morpholinomethyl)-3H-pyrido[3,2-f][1,4]oxazepin-5-one;
(193) 8-(3,3a,4,5,6,6a-Hexahydro-1H-cyclopenta[c]pyrrole-2-carbonyl)-4-[(2R)-3-(3,4-dihydro-1H-isoquinolin-2-yl)-2-hydroxy-propyl]-2,3-dihydro-1,4-benzoxazepin-5-one;
(194) 4-[(2R)-3-(3,4-dihydro-1H-isoquinolin-2-yl)-2-hydroxy-propyl]-8-(3-fluoropiperidine-1-carbonyl)-2,3-dihydro-1,4-benzoxazepin-5-one;
(195) 8-(2,2-Difluoromorpholine-4-carbonyl)-4-[(2R)-3-(3,4-dihydro-1H-isoquinolin-2-yl)-2-hydroxy-propyl]-2,3-dihydro-1,4-benzoxazepin-5-one;
(196) 8-(4,4-Difluoropiperidine-1-carbonyl)-4-[(2R)-3-(3,4-dihydro-1H-isoquinolin-2-yl)-2-hydroxy-propyl]-2,3-dihydro-1,4-benzoxazepin-5-one;
(197) 4-[(2R)-3-(3,4-dihydro-1H-isoquinolin-2-yl)-2-hydroxy-propyl]-2,2-dimethyl-8-(1-piperidylmethyl)-3H-pyrido[3,2-f][1,4]oxazepin-5-one;
(198) 4-[(2R)-3-(3,4-dihydro-1H-isoquinolin-2-yl)-2-hydroxypropyl]-8-[4-[methyl(oxetan-3-yl)amino]cyclohexoxy]-2,3-dihydro-1,4-benzoxazepin-5-one;
(199) 4-[(2R)-3-(3,4-dihydro-1H-isoquinolin-2-yl)-2-hydroxypropyl]-8-[4-[2-fluoroethyl(methyl)amino]cyclohexoxy]-2,3-dihydro-1,4-benzoxazepin-5-one;
(200) (2S)-4-[(2R)-3-(3,4-dihydro-1H-isoquinolin-2-yl)-2-hydroxy-propyl]-8-[(1-ethanethioyl-4-piperidyl)oxy]-2-methyl-2,3-dihydro-1,4-benzoxazepin-5-one;
(201) (2S)-4-[(2R)-3-(3,4-dihydro-1H-isoquinolin-2-yl)-2-hydroxy-propyl]-8-[1-(2-fluoroethyl)azetidin-3-yl]oxy-2-methyl-2,3-dihydro-1,4-benzoxazepin-5-one;
(202) (2S)-4-[(2R)-3-(3,4-dihydro-1H-isoquinolin-2-yl)-2-hydroxy-propyl]-8-[1-(2-fluoroethyl)-4-piperidyl]oxy-2-methyl-2,3-dihydro-1,4-benzoxazepin-5-one;
(203) 4-[(2R)-3-(3,4-dihydro-1H-isoquinolin-2-yl)-2-hydroxy-propyl]-2,2-dimethyl-8-(3-oxa-8-azabicyclo[3.2.1]octane-8-carbonyl)-3H-1,4-benzoxazepin-5-one;
(204) 4-[(2R)-3-(3,4-dihydro-1H-isoquinolin-2-yl)-2-hydroxy-propyl]-2,2-dimethyl-8-(2-oxa-5-azabicyclo[2.2.1]heptane-5-carbonyl)-3H-1,4-benzoxazepin-5-one;
(205) 4-[(2R)-3-(3,4-dihydro-1H-isoquinolin-2-yl)-2-hydroxy-propyl]-8-(3,5-dimethylmorpholine-4-carbonyl)-2,2-dimethyl-3H-1,4-benzoxazepin-5-one;
(206) 4-[(2R)-3-(3,4-dihydro-1H-isoquinolin-2-yl)-2-hydroxy-propyl]-8-[(3-fluoro-1-methyl-4-piperidyl)oxy]-2,3-dihydro-1,4-benzoxazepin-5-one;
(207) 4-[(2R)-3-(3,4-dihydro-1H-isoquinolin-2-yl)-2-hydroxy-propyl]-8-[[3-fluoro-1-(2-fluoroethyl)-4-piperidyl]oxy]-2,3-dihydro-1,4-benzoxazepin-5-one;
(208) 4-[(2R)-3-(3,4-dihydro-1H-isoquinolin-2-yl)-2-hydroxy-propyl]-8-[[3-fluoro-1-(2-hydroxyethyl)-4-piperidyl]oxy]-2,3-dihydro-1,4-benzoxazepin-5-one;
(209) 4-[(2R)-3-(3,4-dihydro-1H-isoquinolin-2-yl)-2-hydroxy-propyl]-8-(3,4-dihydro-1H-pyrrolo[1,2-a]pyrazine-2-carbonyl)-2,2-dimethyl-3H-1,4-benzoxazepin-5-one;
(210) 8-(3,4,6,7,8,8a-Hexahydro-1H-pyrrolo[1,2-a]pyrazine-2-carbonyl)-4-[(2R)-3-(3,4-dihydro-1H-isoquinolin-2-yl)-2-hydroxy-propyl]-2,2-dimethyl-3H-1,4-benzoxazepin-5-one;
(211) 4-[(2R)-3-(3,4-dihydro-1H-isoquinolin-2-yl)-2-hydroxy-propyl]-8-(5-fluoropyrimidin-2-yl)oxy-2,3-dihydro-1,4-benzoxazepin-5-one;
(212) 4-[(2R)-3-(3,4-dihydro-1H-isoquinolin-2-yl)-2-hydroxy-propyl]-8-[[1-(2-hydroxypropyl)-4-piperidyl]oxy]-2,3-dihydro-1,4-benzoxazepin-5-one;
(213) 4-[(2R)-3-(3,4-dihydro-1H-isoquinolin-2-yl)-2-hydroxy-propyl]-8-[(1-methyl-3-piperidyl)oxy]-2,3-dihydro-1,4-benzoxazepin-5-one;
(214) 4-[(2R)-3-(3,4-dihydro-1H-isoquinolin-2-yl)-2-hydroxy-propyl]-8-propanoyl-2,3-dihydro-1,4-benzoxazepin-5-one;
(215) 4-[(2R)-3-(3,4-dihydro-1H-isoquinolin-2-yl)-2-hydroxy-propyl]-8-[[8-(2-fluoroethyl)-8-azabicyclo[3.2.1]octan-3-yl]oxy]-2,3-dihydro-1,4-benzoxazepin-5-one;
(216) 8-(Cyclopropanecarbonyl)-4-[(2R)-3-(3,4-dihydro-1H-isoquinolin-2-yl)-2-hydroxy-propyl]-2,3-dihydro-1,4-benzoxazepin-5-one;
(217) 8-(Cyclopentanecarbonyl)-4-[(2R)-3-(3,4-dihydro-1H-isoquinolin-2-yl)-2-hydroxy-propyl]-2,3-dihydro-1,4-benzoxazepin-5-one;
(218) 4-[(2R)-3-(3,4-dihydro-1H-isoquinolin-2-yl)-2-hydroxypropyl]-8-[(1-methyl-3-piperidyl)oxy]-2,3-dihydro-1,4-benzoxazepin-5-one;
(219) (2R)-4-[(2R)-3-(3,4-dihydro-1H-isoquinolin-2-yl)-2-hydroxy-propyl]-8-[[1-(2-hydroxyethyl)-4-piperidyl]oxy]-2-methyl-2,3-dihydro-1,4-benzoxazepin-5-one;
(220) (2R)-4-[(2R)-3-(3,4-dihydro-1H-isoquinolin-2-yl)-2-hydroxy-propyl]-8-[[1-(2-hydroxy-2-methyl-propyl)-4-piperidyl]oxy]-2-methyl-2,3-dihydro-1,4-benzoxazepin-5-one;
(221) 4-[(2R)-3-(3,4-dihydro-1H-isoquinolin-2-yl)-2-hydroxy-propyl]-8-(1-morpholinoethyl)-2,3-dihydro-1,4-benzoxazepin-5-one;
(222) 4-[(2R)-3-(3,4-dihydro-1H-isoquinolin-2-yl)-2-hydroxy-propyl]-8-[1-(2-oxa-6-azaspiro[3.3]heptan-6-yl)ethyl]-2,3-dihydro-1,4-benzoxazepin-5-one;
(223) 4-[(2R)-3-(3,4-dihydro-1H-isoquinolin-2-yl)-2-hydroxy-propyl]-8-(3-hydroxyprop-1-ynyl)-2,3-dihydro-1,4-benzoxazepin-5-one;
(224) (2R)-4-[(2R)-3-(3,4-dihydro-1H-isoquinolin-2-yl)-2-hydroxy-propyl]-8-[[1-(2-methoxyethyl)-4-piperidyl]oxy]-2-methyl-2,3-dihydro-1,4-benzoxazepin-5-one;
(225) 4-[(2R)-3-(3,4-dihydro-1H-isoquinolin-2-yl)-2-hydroxy-propyl]-8-(4-pyridyl)-2,3-dihydro-1,4-benzoxazepin-5-one;
(226) 4-[(2R)-3-(3,4-dihydro-1H-isoquinolin-2-yl)-2-hydroxy-propyl]-8-[2-(4-pyridyl)ethynyl]-2,3-dihydro-1,4-benzoxazepin-5-one;
(227) 4-[(2R)-3-(3,4-dihydro-1H-isoquinolin-2-yl)-2-hydroxy-propyl]-8-[2-(3-pyridyl)ethynyl]-2,3-dihydro-1,4-benzoxazepin-5-one;
(228) 4-[(2R)-3-(3,4-dihydro-1H-isoquinolin-2-yl)-2-hydroxy-propyl]-8-(3-hydroxybut-1-ynyl)-2,3-dihydro-1,4-benzoxazepin-5-one;
(229) 4-[(2R)-3-(3,4-dihydro-1H-isoquinolin-2-yl)-2-hydroxy-propyl]-8-[3-(methylamino)prop-1-ynyl]-2,3-dihydro-1,4-benzoxazepin-5-one;
(230) 4-[(2R)-3-(3,4-dihydro-1H-isoquinolin-2-yl)-2-hydroxy-propyl]-8-(3-pyridyl)-2,3-dihydro-1,4-benzoxazepin-5-one;
(231) 4-[(2R)-3-(3,4-dihydro-1H-isoquinolin-2-yl)-2-hydroxy-propyl]-8-(1,3-dimethylpyrazol-4-yl)-2,3-dihydro-1,4-benzoxazepin-5-one;
(232) 4-[(2R)-3-(3,4-dihydro-1H-isoquinolin-2-yl)-2-hydroxy-propyl]-8-(1-methylpyrazol-4-yl)-2,3-dihydro-1,4-benzoxazepin-5-one;
(233) 4-[(2R)-3-(3,4-dihydro-1H-isoquinolin-2-yl)-2-hydroxy-propyl]-8-(1,5-dimethylpyrazol-4-yl)-2,3-dihydro-1,4-benzoxazepin-5-one;
(234) 4-[(2R)-3-(3,4-dihydro-1H-isoquinolin-2-yl)-2-hydroxy-propyl]-8-[[1-(2-hydroxyethyl)-4-piperidyl]oxy]-2,2-dimethyl-3H-pyrido[3,2-f][1,4]oxazepin-5-one;
(235) 4-[(2R)-3-(3,4-dihydro-1H-isoquinolin-2-yl)-2-hydroxy-propyl]-2,2-dimethyl-8-[[(3R)-1-methyl-3-piperidyl]oxy]-3H-pyrido[3,2-f][1,4]oxazepin-5-one;
(236) 4-[(2R)-3-(3,4-dihydro-1H-isoquinolin-2-yl)-2-hydroxy-propyl]-8-[[(1-(2-fluoroethyl)-3-piperidyl)oxy)-2,3-dihydro-1,4-benzoxazepin-5-one;
(237) 4-[(2R)-3-(3,4-dihydro-1H-isoquinolin-2-yl)-2-hydroxy-propyl]-8-[[(1-(2-hydroxyethyl)-3-piperidyl)oxy)-2,3-dihydro-1,4-benzoxazepin-5-one;
(238) 4-[(2R)-3-(3,4-dihydro-1H-isoquinolin-2-yl)-2-hydroxy-propyl]-8-[[1-(2-hydroxy-2-methyl-propyl)-4-piperidyl]oxy]-2,3-dihydro-1,4-benzoxazepin-5-one;
(239) 4-[(2R)-3-(3,4-dihydro-1H-isoquinolin-2-yl)-2-hydroxy-propyl]-8-(6-fluoro-2-pyridyl)-2,3-dihydro-1,4-benzoxazepin-5-one;
(240) (2R)-4-[(2R)-3-(3,4-dihydro-1H-isoquinolin-2-yl)-2-hydroxy-propyl]-8-[[1-(2-ethoxyethyl)-4-piperidyl]oxy]-2-methyl-2,3-dihydro-1,4-benzoxazepin-5-one;
(241) (2R)-4-[(2R)-3-(3,4-dihydro-1H-isoquinolin-2-yl)-2-hydroxy-propyl]-8-[[8-(2-hydroxyethyl)-8-azabicyclo[3.2.1]octan-3-yl]oxy]-2-methyl-2,3-dihydro-1,4-benzoxazepin-5-one;
(242) (2R)-4-[(2R)-3-(3,4-dihydro-1H-isoquinolin-2-yl)-2-hydroxy-propyl]-8-[[8-(2-fluoroethyl)-8-azabicyclo[3.2.1]octan-3-yl]oxy]-2-methyl-2,3-dihydro-1,4-benzoxazepin-5-one;
(243) 4-[(2R)-3-(3,4-dihydro-1H-isoquinolin-2-yl)-2-hydroxy-propyl]-8-[[1-(2-methoxyethyl)-4-piperidyl]oxy]-2,3-dihydro-1,4-benzoxazepin-5-one;
(244) 4-[(2R)-3-(3,4-dihydro-1H-isoquinolin-2-yl)-2-hydroxy-propyl]-8-(6-methoxy-2-pyridyl)-2,3-dihydro-1,4-benzoxazepin-5-one;
(245) (2R)-4-[(2R)-3-(3,4-dihydro-1H-isoquinolin-2-yl)-2-hydroxy-propyl]-8-[[1-(2-hydroxypropyl)-4-piperidyl]oxy]-2-methyl-2,3-dihydro-1,4-benzoxazepin-5-one;
(246) 4-[(2R)-3-(3,4-dihydro-1H-isoquinolin-2-yl)-2-hydroxy-propyl]-2,2-dimethyl-8-(1-morpholinoethyl)-3H-pyrido[3,2-f][1,4]oxazepin-5-one;
(247) 4-[(2R)-3-(3,4-dihydro-1H-isoquinolin-2-yl)-2-hydroxy-propyl]-2,2-dimethyl-8-[1-(2-oxa-6-azaspiro[3.3]heptan-6-yl)ethyl]-3H-pyrido[3,2-f][1,4]oxazepin-5-one;
(248) 4-[(2R)-3-(3,4-dihydro-1H-isoquinolin-2-yl)-2-hydroxy-propyl]-8-[[1-(2-hydroxypropyl)-4-piperidyl]oxy]-2,2-dimethyl-3H-pyrido[3,2-f][1,4]oxazepin-5-one;
(249) 4-[(2R)-3-(3,4-dihydro-1H-isoquinolin-2-yl)-2-hydroxypropyl]-8-(3,4-dihydro-1H-pyrrolo[1,2-a]pyrazin-2-ylmethyl)-2,3-dihydro-1,4-benzoxazepin-5-one;
(250) (2R)-4-[(2R)-3-(3,4-dihydro-1H-isoquinolin-2-yl)-2-hydroxypropyl]-2-methyl-8-(2-oxa-6-azaspiro[3.3]heptan-6-ylmethyl)-2,3-dihydro-1,4-benzoxazepin-5-one;
(251) 4-[(2R)-3-(3,4-dihydro-1H-isoquinolin-2-yl)-2-hydroxypropyl]-8-(2-oxa-7-azaspiro[3.5]nonan-7-ylmethyl)-2,3-dihydro-1,4-benzoxazepin-5-one;
(252) (2R)-4-[(2R)-3-(3,4-dihydro-1H-isoquinolin-2-yl)-2-hydroxypropyl]-2-methyl-8-(2-oxa-7-azaspiro[3.5]nonan-7-ylmethyl)-2,3-dihydro-1,4-benzoxazepin-5-one;
(253) (2R)-4-[(2R)-3-(3,4-dihydro-1H-isoquinolin-2-yl)-2-hydroxy-propyl]-8-[[(3R,4R)-3-fluoro-1-(2-hydroxyethyl)-4-piperidyl]oxy]-2-methyl-2,3-dihydro-1,4-benzoxazepin-5-one 2 hydrochloride;
(254) (2R)-4-[(2R)-3-(3,4-dihydro-1H-isoquinolin-2-yl)-2-hydroxy-propyl]-8-[[(3R,4R)-3-fluoro-1-(2-hydroxypropyl)-4-piperidyl]oxy]-2-methyl-2,3-dihydro-1,4-benzoxazepin-5-one 2 hydrochloride;
(255) (2R)-4-[(2R)-3-(3,4-dihydro-1H-isoquinolin-2-yl)-2-hydroxy-propyl]-8-[[(3R,4R)-3-fluoro-1-(2-hydroxyethyl)-4-piperidyl]oxy]-2-methyl-2,3-dihydro-1,4-benzoxazepin-5-one 2 hydrochloride;
(256) 8-[[3,3-Difluoro-1-(2-hydroxyethyl)-4-piperidyl]oxy]-4-[(2R)-3-(3,4-dihydro-1H-isoquinolin-2-yl)-2-hydroxy-propyl]-2,2-dimethyl-3H-pyrido[3,2-f][1,4]oxazepin-5-one;
(257) 4-[(2R)-3-(3,4-dihydro-1H-isoquinolin-2-yl)-2-hydroxypropyl]-8-[1-(1-piperidyl)ethyl]-2,3-dihydro-1,4-benzoxazepin-5-one;
(258) 4-[(2R)-3-(3,4-dihydro-1H-isoquinolin-2-yl)-2-hydroxypropyl]-8-[1-(4-hydroxy-1-piperidyl)ethyl]-2,3-dihydro-1,4-benzoxazepin-5-one;
(259) 4-[(2R)-3-(3,4-dihydro-1H-isoquinolin-2-yl)-2-hydroxypropyl]-8-[1-(4-methoxy-1-piperidyl)ethyl]-2,3-dihydro-1,4-benzoxazepin-5-one;
(260) 4-[(2R)-3-(3,4-dihydro-1H-isoquinolin-2-yl)-2-hydroxypropyl]-8-[1-(3-hydroxy-3-methyl-pyrrolidin-1-yl)ethyl]-2,3-dihydro-1,4-benzoxazepin-5-one;
(261) (2R)-4-[(2R)-3-(3,4-dihydro-1H-isoquinolin-2-yl)-2-hydroxy-propyl]-8-[[1-(2-hydroxyethyl)-4-piperidyl]oxy]-2-methyl-2,3-dihydro-1,4-benzoxazepin-5-one 2 hydrochloride;
(262) 4-[(2R)-3-(3,4-dihydro-1H-isoquinolin-2-yl)-2-hydroxy-propyl]-8-[[1-(2-methoxyethyl)-4-piperidyl]oxy]-2,2-dimethyl-3H-pyrido[3,2-f][1,4]oxazepin-5-one;
(263) 4-[(2R)-3-(3,4-dihydro-1H-isoquinolin-2-yl)-2-hydroxy-propyl]-8-[(3S)-1-(oxetan-3-yl)pyrrolidin-3-yl]oxy-2,3-dihydro-1,4-benzoxazepin-5-one;
(264) (2R)-4-[(2R)-3-(3,4-dihydro-1H-isoquinolin-2-yl)-2-hydroxy-propyl]-2-methyl-8-[(3S)-1-methylpyrrolidin-3-yl]oxy-2,3-dihydro-1,4-benzoxazepin-5-one;
(265) 4-[(2R)-3-(3,4-dihydro-1H-isoquinolin-2-yl)-2-hydroxy-propyl]-8-[(3R)-tetrahydrofuran-3-yl]oxy-2,3-dihydro-1,4-benzoxazepin-5-one;
(266) 4-[(2R)-3-(3,4-dihydro-1H-isoquinolin-2-yl)-2-hydroxy-propyl]-8-[(3S)-tetrahydrofuran-3-yl]oxy-2,3-dihydro-1,4-benzoxazepine-5-one;
(267) (2R)-4-[(2R)-3-(3,4-dihydro-1H-isoquinolin-2-yl)-2-hydroxy-propyl]-8-[[1-[(2S)-2-hydroxypropyl]-4-piperidyl]oxy]-2-methyl-2,3-dihydro-1,4-benzoxazepin-5-one;
(268) (2R)-4-[(2R)-3-(3,4-dihydro-1H-isoquinolin-2-yl)-2-hydroxy-propyl]-8-[[1-[(2R)-2-hydroxypropyl]-4-piperidyl]oxy]-2-methyl-2,3-dihydro-1,4-benzoxazepin-5-one;
(269) 8-[Cyclopropyl(hydroxy)methyl]-4-[(2R)-3-(3,4-dihydro-1H-isoquinolin-2-yl)-2-hydroxy-propyl]-2,3-dihydro-1,4-benzoxazepin-5-one;
(270) 8-[Cyclopentyl(hydroxy)methyl]-4-[(2R)-3-(3,4-dihydro-1H-isoquinolin-2-yl)-2-hydroxy-propyl]-2,3-dihydro-1,4-benzoxazepin-5-one;
(271) 4-[(2R)-3-(3,4-dihydro-1H-isoquinolin-2-yl)-2-hydroxy-propyl]-8-[(3R)-1-(oxetan-3-yl)pyrrolidin-3-yl]oxy-2,3-dihydro-1,4-benzoxazepin-5-one;
(272) 4-[(2R)-3-(3,4-dihydro-1H-isoquinolin-2-yl)-2-hydroxy-propyl]-8-[(3R)-1-(2-methoxyethyl)pyrrolidin-3-yl]oxy-2,3-dihydro-1,4-benzoxazepin-5-one;
(273) (2R)-4-[(2R)-3-(3,4-dihydro-1H-isoquinolin-2-yl)-2-hydroxy-propyl]-2-methyl-8-[(3R)-1-methylpyrrolidin-3-yl]oxy-2,3-dihydro-1,4-benzoxazepin-5-one;
(274) (2R)-4-[(2R)-3-(3,4-dihydro-1H-isoquinolin-2-yl)-2-hydroxy-propyl]-8-[(3R)-1-(2-methoxyethyl)pyrrolidin-3-yl]oxy-2-methyl-2,3-dihydro-1,4-benzoxazepin-5-one;
(275) 4-[(2R)-3-(3,4-dihydro-1H-isoquinolin-2-yl)-2-hydroxy-propyl]-8-[[1-(3,3,3-trifluoro-2-hydroxy-propyl)-4-piperidyl]oxy]-2,3-dihydro-1,4-benzoxazepin-5-one;
(276) (2R)-4-[(2R)-3-(3,4-dihydro-1H-isoquinolin-2-yl)-2-hydroxy-propyl]-2-methyl-8-[[1-(3,3,3-trifluoro-2-hydroxy-propyl)-4-piperidyl]oxy]-2,3-dihydro-1,4-benzoxazepin-5-one; and
(277) (2R)-4-[(2R)-3-(3,4-dihydro-1H-isoquinolin-2-yl)-2-hydroxypropyl]-2-methyl-8-[(3R)-1-(oxetan-3-yl)pyrrolidin-3-yl]oxy-2,3-dihydro-1,4-benzoxazepin-5-one.

18. The pharmaceutical combination of claim 1, wherein the PRMT5 inhibitor of the Chemical Formula 1 is at least one selected from the group consisting of:
4-[(2R)-3-(3,4-dihydro-1H-isoquinolin-2-yl)-2-hydroxy-propyl]-8-[(1-methyl-4-piperidyl)oxy]-2,3-dihydro-1,4-benzoxazepin-5-one;
4-[(2R)-3-(3,4-dihydro-1H-isoquinolin-2-yl)-2-hydroxy-propyl]-2,2-dimethyl-8-[(1-methyl-4-piperidyl)oxy]-3H-1,4-benzoxazepin-5-one; and
4-[(2R)-3-(3,4-dihydro-1H-isoquinolin-2-yl)-2-hydroxy-propyl]-8-[(3R)-1-(oxetan-3-yl)pyrrolidin-3-yl]oxy-2,3-dihydro-1,4-benzoxazepin-5-one.

19. The pharmaceutical combination of claim 1, wherein the DNA damage inducing substance is selected from the group consisting of alkylating agents, anti-metabolites, topoisomerase inhibitors, microtubule inhibitors, and poly (ADP-ribose) polymerase (PARP) inhibitors.

20. The pharmaceutical combination of claim 19, wherein the alkylating agent is selected from the group consisting of cisplatin, carboplatin, oxaliplatin, mechlorethamine, cyclophosphamide, ifosfamide, melphalan, chlorambucil, carmustine (BCNU), lomustine (CCNU), nimustine, altretamine, busulfan, dacarbazine, procarbazine, temozolomide, thiotepa, and lurbinectedin;
wherein the anti-metabolite is selected from the group consisting of fluorouracil (5-FU), capecitabine, cytarabine, gemcitabine, methotrexate, and mercaptopurine (6-MP);
wherein the topoisomerase inhibitor is selected from the group consisting of etoposide, teniposide, topotecan, irinotecan, SN-38, dactinomycin, doxorubicin, daunorubicin, mitomycin, and bleomycin;
wherein the microtubule inhibitor is selected from the group consisting of vinblastine, vincristine, vinorelbine, paclitaxel, and docetaxel; or
wherein the poly (ADP-ribose) polymerase (PARP) inhibitor is at least one selected from the group consisting of olaparib, veliparib, niraparib, pamiparib, talazoparib, rucaparib, CEP-9722, E7016 (GPI 21016), fluzoparib, AZD5305, and AZD9574.

21.-31. (canceled)

32. The pharmaceutical combination of claim 1, wherein the content ratio of the PRMT5 inhibitor of Chemical Formula 1 and the DNA damage inducing substance is in the range of 1:3000 to 1:0.00015 by weight.

33. A method of treating or preventing cancer in a subject, comprising
administering the pharmaceutical combination of claim 1 to the subject.

34. The method of claim 33, wherein the subject is a multidrug resistance (MDR) subject; the subject exhibits resistance or low responsiveness to a DNA damage inducing substance; or the subject is either HRD (Homologous recombination deficiency)-positive or HRD-negative.

35. The method of claim 33, wherein the cancer is at least one selected from the group consisting of acoustic neuroma, adenocarcinoma, adrenal cancer, anal cancer, angiosarcoma, benign monoclonal gammopathy, cholangiocarcinoma, bladder cancer, breast cancer, brain cancer, lymphoma, multiple myeloma, lacrimal gland tumor, bronchial cancer, cervical cancer, craniopharyngioma, colorectal cancer, epithelial carcinoma, ependymoma, adenoid cystic carcinoma (ACC), endothelial sarcoma, endometrial cancer, esophageal cancer, Barrett's adenocarcinoma, Ewing's sarcoma, eye cancer, gallbladder cancer, gastric cancer, colon cancer, gastrointestinal stromal tumor (GIST), head and neck cancer, oral cancer (OSCC), throat cancer, hematologic cancer, hemangioblastoma, inflammatory myofibroblastic tumor, immunocyte-associated amyloidosis, kidney cancer, renal cell carcinoma, liver cancer, lung cancer, melanoma, uveal melanoma, acute myeloid leukemia (AML), myelodysplastic syndrome (MDS), mesothelioma, myeloproliferative disorder (MPD), chronic idiopathic myelofibrosis, chronic myeloid leukemia (CML), chronic neutrophilic leukemia (CNL), neuroblastoma, neurofibroma, neuroendocrine cancer, osteosarcoma, ovarian cancer, papillary adenocarcinoma, pancreatic cancer, penile cancer, prostate cancer, rectal cancer, rhabdomyosarcoma, salivary gland cancer, skin cancer, small intestinal cancer, soft tissue sarcoma, thyroid cancer, urethral cancer, vaginal cancer and vulvar cancer.

36. The method of claim 33, wherein the brain cancer is at least one selected from the group consisting of meningioma, glioma, medulloblastoma, glioblastoma and brain metastasis;
wherein the lung cancer is non-small cell lung cancer (NSCLC) or small cell lung cancer (SCLC); or
wherein the breast cancer or the ovarian cancer is one selected from the group consisting of BRCA1 wild type and BRCA2 wild type cancer, BRCA1 wild type and BRCA2 mutant cancer, BRCA1 mutant and BRCA2 wild type cancer, and BRCA1 mutant and BRCA2 mutant cancer.
